(19) United States
(12) Patent Application Publication
(10) Pub. No.: US 2026/0286326 A1
(43) Pub. Date: Sep. 24, 2026

(54) NUCLEASE COMPLEX MODIFYING THE DIRECTION OF NUCLEOTIDE DEGRADATION FROM 3'-5' EXONUCLEASE TO INCLUDE ENDONUCLEASE DEPENDING ON THE RATIO OF RecJ3/4 AND aRNase J

(71) Applicant: UNIVERSITY OF FLORIDA RESEARCH FOUNDATION, INCORPORATED, Gainesville, FL (US)

(72) Inventors: Julie A. MAUPIN, Gainesville, FL (US); Huiyong JIA, Gainesville, FL (US); Swathi DANTULURI, Gainesville, FL (US)

(21) Appl. No.: 19/475,870

(22) PCT Filed: Apr. 29, 2024
(86) PCT No.: PCT/US2024/026765

§ 371 Date: Oct. 16, 2025

Related U.S. Application Data
(60) Provisional application No. 63/462,626, filed on Apr. 28, 2023.

(51) Int. Cl. C12N 9/22; C12N 15/74

(52) U.S. Cl. CPC C12N 9/22; C12N 15/74; C07K 2319/21; C07K 2319/22

(57) ABSTRACT

Here a new type of nuclease complex composed of RecJ3/4 subcomplex and aRNase J from H. volcanii is disclosed. This complex is found to transiently associate with Cdc48a and Uhls in the presence of ATP. As the ratio of RecJ3/4 subcomplex to aRNase J is increased in the reconstitution of this complex in vitro, the activities of aRNascJ arc found to shift from endonuclease and 5′-3′ exonuclease to primarily 3′-5′ exonuclease with preference for RNA to ssDNA. Phenotypic analysis of mutant strains reveals aRNase J is essential and Cdc48a, RecJ3, and RecJ4 are important for recovery of cells from DNA damaging agents including those that introduce double-strand breaks (DSBs). The disclosure here suggests a novel nuclease system that can control degradation process and direction of RNA as well as ssDNA in vitro.

### STATEMENT OF FEDERAL FUNDING

This invention was made with government support under Grant No. GM057498 awarded by The National Institutes of Health. The government has certain rights in the invention.

### BACKGROUND

Nucleases are enzymes that are important in cell biology and are often strictly regulated to avoid the uncontrolled degradation of DNA and RNA. Nucleases are used in many cellular processes, including DNA replication, DNA recombination and repair, RNA maturation, RNA processing, RNA interference, nutrient regeneration, and cell death (1). Nucleases can regulate mRNA abundance in response to environmental, developmental, and metabolic cues (2, 3). They can also maintain RNA quality by preventing the damaging effects of aberrant non-coding RNA accumulation or defective mRNA translation (4).

Interestingly, in addition to DNases, RNases are recently found important in DNA repair, with associated components identified in the proximity of double-stranded (ds) DNA breaks (5, 6). Ribonucleases (RNases) mediate the processing, turnover, and quality control of RNA. RNases are generally divided into two groups: (i) endonucleases that cleave the RNA internally and (ii) exonucleases that cleave the RNA from the 3′ or 5′ ends. Some RNases have both types of activities [e.g., (7-9)]. These catalytic activities are controlled to ensure the proper selection and timing of the RNA species to be hydrolyzed (10). RNA degradation is regulated by various factors including mechanisms of post-transcriptional, post-translational, and trans-acting protein or non-coding RNA inhibitor control (10). RNases can also be associated in multi-subunit complexes and/or localized in the cell to prevent the uncontrolled degradation of RNA (10).

Many archaea encode a multisubunit complex that appears central to RNA hydrolysis and shares an evolutionary relationship to the eukaryotic RNA exosome. The core of this archaeal RNA-degrading machine (RNA exosome) is composed of Rrp41 (3′-5′ exoribonuclease and poly-RNA tailing), Rrp42 (non-catalytic scaffold), Rrp4 (RNA-binding), Cs14 (RNA presentation), and DnaG (DNA primase) (11). Additional subunits include Nop5 (rRNA/tRNA 2′-O-methyl-transferase) (12), aRNase J, and a Ski2-like RNA helicase (13). Interestingly, a large group of curyarchacota, including the halophilic and methanogenic archaca, are missing most subunits (i.e., Rrp41, Rrp42, Rrp4 and Cs14) of the RNA exosome core. A nuclease complex that is central to RNA turnover in this group of euryarchaeota remains to be determined.

### SUMMARY

RNase J proteins are widespread in prokaryotes and key members of the β-CASP family of metallo-β-lactamases. In archaea, these enzymes function as 5′-3′ exonucleases as revealed by the study of Pyrococcus abyssi Pab-aRNase J (13, 14), Thermococcus kodakarensis Tko-aRNase J (14), Methanolobus psychrophilus Mpy-aRNase J (15), and Methanocaldococcus jannaschii Mja-aRNase J1 (16). RNA unwinding activity is also observed for Mpy-aRNase J (17). By comparison, bacterial RNase J enzymes function as 5′-3′ exo- and/or endoribonucleases (7, 18-23) with differences in metal ion binding, oligomerization, C-terminal extensions, and RNA interactions suggested to determine these catalytic activities (7, 18-24).

RecJ homologs are conserved in all domains of life and found to hydrolyze DNA and/or RNA in the 3′-5′ and/or 5′-3′ direction (25-27). RecJ enzymes function in DNA repair and recombination, nucleotide recycling, stress tolerance, and the final stages of chromosome duplication (28-33). In bacteria, such as Escherichia coli, RecJ is a single-stranded (ss) DNA-specific 5′-3′ exonuclease that is processive (26, 27), while other bacterial RecJ enzymes are identified that cleave dsDNA (34). Archaea have an expanded repertoire of RecJ homologs related to bacterial RecJ and eukaryal Cdc45, the latter a non-catalytic subunit of the CMG complex. The CMG complex, essential to DNA replication in eukaryotes, is composed of Cdc45, minichromosome maintenance (MCM) helicase, and GINS (from the Japanese go-ichi-ni-san meaning 5-1-2-3, after the four related subunits of the complex Sld5, Psf1, Psf2, and Psf3) (25, 35-37). The archaeal RecJ homolog GAN (GINS-associated nuclease) functions as a DNA/RNA exonuclease that associates with GINS and the MCM helicase (38-41). Moreover, the Hef-associated nuclease (HAN) (25) is an archaeal RecJ homolog that interacts with Hef, an endonuclease that cleaves DNA with various branched structures (42). Hef has an intrinsically disordered region (IDR) that is needed for association with HAN and can bind other proteins including PCNA1 (37).

Haloferax volcanii, the model archaeon of this study, has an aRNase J and four RecJ (RccJ1-4) homologs that cluster into distinct arCOG groups (FIG. 1) (35). RccJ1 of arCOG00427 is closely related to GANs. The other GAN relative, RecJ2, clusters to the arCOG00428 group distinct to the members of the Halobacteria and Nanoarchaeota and is predicted to include inactive nucleases. RecJ3 and RecJ4 are members of the HAN-containing arCOG00429 group, with RecJ4 missing the conserved active site residues. An H. volcanii ΔrecJ3 mutant is characterized and found to be impaired in survival against methyl methanesulfonate (43), a DNA alkylating agent suggested to induce stalled replication forks (44). Based on these features, H. volcanii RecJ3 is annotated as HAN (43) but has yet to be characterized at the biochemical level.

Post-translational modification (PTM) systems regulate mRNA turnover and surveillance as well as DNA replication and repair (45-48). PTMs, such as phosphorylation and the tagging of proteins with ubiquitin (Ub) or Ub-related proteins (e.g., SUMO), are important in orchestrating the progression of protein factors in eukaryotic DNA repair pathways (6, 46, 47). Proteasomes, multi-subunit complexes that recognize Ub-tagged proteins, localize to sites of DNA breaks and are required for effective mRNA surveillance and protein turnover in eukaryotes (45). A Ub-like (Ubl) protein modification system is identified in archaea (49-53) but has yet to be associated with DNA repair/recombination or RNA degradation pathways.

Here, a new type of nuclease complex composed of RecJ3, RecJ4, and aRNase J is reported in H. volcanii, an archaeon lacking an RNA exosome. This complex was identified based on its transient ATP-dependent association with the Ubl SAMP1 and AAA-ATPase Cdc48a. The primary activity of the complex was identified as a 3′-5′ exonuclease against RNA and ssDNA. Further dissection of the complex revealed RecJ3/4 to associate in 1:1 molar ratio as a subcomplex with 3′-5′ exonuclease activity and aRNase J to form a homodimer. The aRNase J alone was found to have an endoribonuclease activity not previously observed for other aRNase J homologs. Reconstitution of the aRNase J with RecJ3/4 resulted in a restriction to primarily 3′-5′ exonuclease activity. Phenotypic analysis of mutant strains revealed aRNase J to be essential, while Cdc48a, RccJ3, and RecJ4 were found important for cellular recovery from DNA-damaging agents including those that introduce double-strand breaks (DSBs). These results suggest that RecJ3 and RecJ4 are associated with DNA repair and regulate the nuclease activities of aRNase J.

In summary, nucleases are critical for various cellular processes including DNA replication and repair. Here, a dynamic type of nuclease complex is newly identified in the archacon Haloferax volcanii, which is missing the canonical RNA exosome. The complex, composed of RecJ3, RecJ4, and aRNase J, functions primarily as a 3′-5′ exonuclease, and was discovered through its ATP-dependent association with the ubiquitin-like SAMP1 and Cdc48a. aRNase J alone forms a homodimer that has endonuclease function and, thus, is not restricted to 5′-3′ exonuclease activity typical of other aRNase J enzymes. RecJ3/4 appears to suppress, alter, and/or outcompete the nuclease activities of aRNase J. While aRNase J is essential for growth, RecJ3/4, Cdc48a, and SAMPs are important for recovery against DNA damage. These biological distinctions may correlate with the regulated nuclease activity of aRNase J in the RecJ3/4-aRNaseJ complex.

Therefore, in this disclosure is provided a nuclease complex system comprising a nuclease and a set of proteins for controlling the nuclease to control the degradation of a single stranded nucleic acid, wherein the nuclease is aRNase J, wherein the set of nuclease-controlling proteins comprises RecJ3 and RecJ4, and wherein the single stranded nucleic acid is RNA or ssDNA.

aRNase J has an amino acid sequence of SEQ ID NO:5 encoded from a nucleotide sequence SEQ ID NO:6, or any RNase with sequence identity above 90% thereof can be used. aRNase J forms a homodimer, and aRNase J is isolated from archaea that do not encode a canonical RNA exosome, selected from Euryarchaeota comprising thermococcales, methanosarcinales, methanomicrobiales, haloarchaea and certain methanogens, and optionally Haloferax volcanii.

RecJ3 and RecJ4 complex form a heterodimer at an equimolar ratio. RecJ3 comprises an amino acid sequence of SEQ ID NO:1 encoded from a nucleotide sequence of SEQ ID NO:2 or any sequence with sequence identity above 90% thereof. RecJ4 comprises an amino acid sequence of SEQ ID NO:3 encoded from a nucleotide sequence of SEQ ID NO:4, or any sequence with sequence identity above 90% thereof. RecJ3 and RecJ4 are isolated from archaea, selected from Euryarchaeota comprising thermococcales, methanosarcinales, methanomicrobiales, haloarchaea and certain methanogens, and optionally H. volcanii. The ratio of RecJ3, RecJ4 and aRNase J is within a range of about 0:0:1 to about 2:2:1, and optionally about 0.5:0.5:1 or about 2:2:1, wherein (i) at the ratio of 0:0:1 of RecJ3, RecJ4, and aRNase J, aRNase J has activity as endonuclease and 5′-3′ exonuclease; (ii) at a low ratio, aRNase J has more activity as endonuclease and 5′-3′ exonuclease than 3′-5′ exonuclease, (iii) at a high ratio, aRNase J has more activity as 3′-5′ exonuclease than endonuclease and 5′-3′ exonuclease; and (iv) at the ratio of 2:2:1, the complex has 3′-5′ exonuclease activity.

For protein purification from recombinant cells, aRNase J optionally has a tag at its C-terminal or N-terminal for protein purification, wherein the tag can be selected from hexa-histidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, and the like, and optionally aRNase J has StrepII tag at its C-terminal.

For protein purification from recombinant cells, RecJ3 and RecJ4 optionally have a tag at its C-terminal or N-terminal, wherein the tag can be selected from hexa-histidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, and the like. Optionally, RecJ3 has His tag at its N terminal, and RecJ4 has StrepII tag at its C terminal.

For in vitro use, a nuclease activity titration kit is provided, comprising RecJ3, RecJ4, and aRNaseJ proteins individually packed in a container as powder or concentrated solution form, which are at least partially purified, wherein the kit further comprises concentrated reaction buffer having pH of about 7.2-7.7, Mg²⁺, Mn²⁺, and high salt to make a final concentration of about 2M NaCl in the reaction solution, reaction stop solution comprising EDTA and/or TPEN, and nuclease activity titration substrates comprising RNAs or ssDNA labeled with 6-carboxyfluorescein (6-FAM) at 5′ end or at 3′ end, and RNAs labeled internally with phosphorothioate and at 3′ end with 6-FAM.

Further, for in vivo expression of the nuclease system comprising aRNase J homodimer and RecJ3/4 heterodimers described above, recombinant expression vectors are provided. One recombinant expression vector or plasmid comprises a nucleic acid strand encoding both RecJ3 and RecJ4, wherein a promoter can be positioned upstream of the nucleic acid strand encoding both RecJ3 and RecJ4, and wherein the promoter is an inducible by tryptophan, IPTG or arabinose. The other recombinant expression vector comprises a nucleic acid strand encoding aRNase J, wherein a promoter, inducible or constitutively active, different from said promoter can be positioned upstream of the nucleic acid strand encoding aRNase J. However, a recombinant expression vector can comprise a nucleic acid strand encoding all three proteins of RecJ3, RecJ4 and aRNaseJ, wherein a promoter can be positioned upstream of the nucleic acid strand encoding both RecJ3 and RecJ4, wherein the promoter is an inducible by tryptophan, IPTG, or arabinose, and another promoter different from said promoter, inducible or constitutively active, can be positioned upstream of the nucleic acid strand encoding aRNaseJ. Further, cells recombinantly engineered to express a heterologous nucleic acid strand encoding RecJ3, RecJ4 and aRNase J, or heterologous nucleic acid strands encoding RecJ3, RecJ4 or aRNase J or a combination thereof are provided, wherein the cell is selected from bacterial, yeast, insect, and mammalian cells.

In some embodiments, a nucleic acid strand encoding aRNase J with a StrepII tag at its C-terminal (aRNase J-StrepII), and a recombinant replication and/or protein expression vector comprising said nucleic acid strand are provided.

In some embodiments, a nucleic acid strand or nucleic acid strands encoding RecJ3 with a His6 tag at its N-terminal (His6-RecJ3) and/or RecJ4 with a StrepII tag at its C-terminal (RecJ4-StrepII), and a recombinant replication and/or protein expression vector or recombinant replication and/or protein expression vectors comprising said nucleic acid strand(s) is/are provided.

Also, in some embodiments, purification methods for those recombinant proteins are provided.

### DESCRIPTION OF THE DRAWINGS

FIG. 1 shows phylogenetic relationship, nucleotidase activities and protein partners of RecJ homologs. Figures from reference (35) (phylogentic relationship) and (25) (display) were merged and modified to include HvoRecJ1-4 and the functional interaction of EcoRecJ with RecQ helicase (21). *none, presumed based on the absence of catalytic active site residues. Hvo, H. volcanii; Tko, Thermococcus kodakarensis; Pfu, Pyrococcus furiosus; Tac, Thermoplasma acidophilum; Mja, Methanocaldococcus jannaschii; Eco, Escherichia coli.

FIG. 2 shows Ubl interactome of H. volcanii including Cdc48a, RecJ3/4 and aRNase J. (A) Strategy to isolate H. volcanii proteins that bind agarose beads charged with monomeric Ubl (vs. BSA) as bait. Cell lysate was from the culture of H. volcanii NH02-pJAM957. (B) Left, Non-reducing SDS-PAGE of H. volcanii proteins that bound BSA- (control) vs. Ubl-decorated beads in the presence of ATP as indicated. Gels were stained with SYPRO Ruby. Vertical bar on right of gel, HMW and LMW (high and low molecular weight) regions of gel excised from BSA- and Ubl-associated lanes for LC-MS/MS analysis. Right, Proteins identified at >99.9% probability and <0.1% false discovery rate (FDR) in the Ubl (vs. BSA) samples (see Dataset S1A, Jia et al., infra). Similar trends were observed in independent experiments. ATP was required for this association. Theor. Mr, theoretical molecular mass based on genome sequence. Input, equal concentration and volume of cell lyate with * indicating common non-specific proteins to the Ubl and BSA control samples.

FIG. 3 shows phenotypic analysis of mutants of the Ubl interactome identified in H. volcanii. (A) aRNase J gene (rnj) appears essential for growth. Conditional depletion of rnj expression by removal of tryptophan from HJ05 (T7-p.tnaA:rnj) alone or with the empty vector compared with complementation by ectopic expression of rnj-strepII. (B) recJ3 and recJ4 genes are important for recovery after treatment with phleomycin, a DSB agent. Hypersensitivity of ΔrecJ3 and ΔrecJ4 mutants to phleomycin (left). Complementation of ΔrecJ3 by ectopic expression of his6-recJ3 and recJ3 (right). (C) Cdc48a impacts recovery after treatment with UV (left) and phleomycin (right).+cdc48a, gene ectopically expressed from strong, constitutive (p.rrn2) promoter on plasmid pJAM1410. 4cdc48a, mutant strain. Panels B-C, ** p-value <0.005 and *p-value <0.05 based on unpaired two-tailed t-test compared with parent strain. SEM (error bars) of at least n=3 independent experiments. Images represent at least n=3 independent experiments. See the Materials and Methods for details. WT, ‘wild-type’ parent strain.

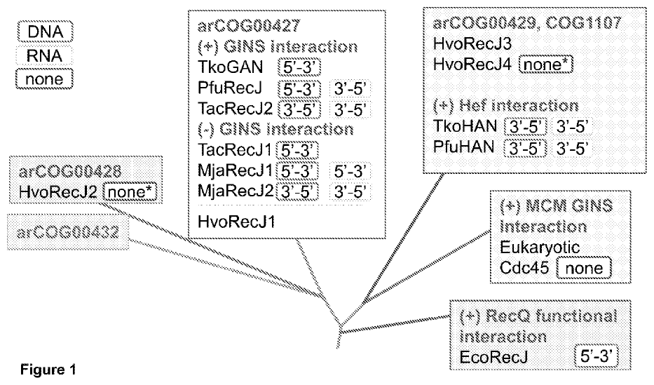

FIG. 4 shows RecJ3/4-aRNase J, RecJ3/4 and aRNase J complexes purified from H. volcanii.
(A) RecJ3/4-aRNase J complex purified by His-Trap. Complex was purified by His-Trap from H. volcanii HJ02-pJAM4251 expressing His6-RecJ3 (lanes 2, 4) compared with empty vector pJAM202c (lanes 1,3). Input (cell lysate, lanes 1-2). Vertical bars indicate regions of gel excised and analyzed by LC-MS/MS (Dataset S1B, Jia et al. infra). Reducing SDS-PAGE gel stained with Bio-Safe Coomassie (Bio-Rad, Hercules, CA, USA).
(B) RecJ3/4-aRNase J complex tandem affinity purification (TAP). Complex was purified from H. volcanii HJ02-pJAM4252 expressing His6-RecJ3 and aRNase J-StrepII (lanes 1, 5). The complex was not purified by TAP from the same strain carrying the empty vector pJAM202c (lanes 3, 7) or from H. volcanii HJ04-pJAM4252, an ΔrecJ4 mutant expressing His6-RecJ3 and aRNasc J-StrepII (lanes 2, 6) or its empty vector control pJAM202c (lanes 4, 8). TAP consisted of HisTrap (lanes 1-4) followed by Strep-Tactin (lanes 5-8). Input (cell lysate, bottom left panel). Left, molecular mass standards. Reducing SDS-PAGE gel stained with SYPRO Ruby. Liquid fractions of samples associated with lanes 5 and 7 were analyzed by LC-MS/MS and AQUA-based MS/MS (Datasets S1C-D, Jia et al.).
(C) Purification of the RecJ3/4 subcomplex and aRNase J homodimer.
i. Chromatogram of RecJ3/4 and aRNase J subcomplexes (black and red, respectively). Observed Mr (molecular mass; indicated) based on size exclusion chromatography (SEC) using a Superdex 200 Increase 10/300 GL chromatography calibrated with molecular mass standards.
ii. aRNase J complex purified from H. volcanii HS01-pJAM1406 (ΔrecJ3 expressing aRNase J-StrepII). Reducing 10% SDS-PAGE gel of cell free extract (CFE, lane 1), StrepTactin fractions (E1-2, lanes 2-3), and SEC fractions (F16-17, lanes 4-5).
iii. RecJ3/4 and RecJ3/4-aRNase J complexes purified from H. volcanii HJ07 (ΔrecJ3 pitA_{Nph} Δrnj expressing His-RecJ3 and RNaseJ-StrepII). Reducing 10% SDS-PAGE gel of cell free extract (CFE, lane 1), Ni-NTA fractions (E1, lane 2), Ni-NTA fractions unbound (FT, lane 3) and bound to StrepTactin (E2, lane 4), and SEC fractions of lane 3 FT (SEC F5-18, lanes 5-18).

FIG. 5 shows nuclease activity of aRNase J homodimer.
(A) aRNase J hydrolysis of RNA and ssDNA oligonucleotides. Reactions (10 μL): 50 mM HEPES (pH 7.5) with 2 M NaCl, 5 mM MnCl₂, 5 mM MgCl₂, 1 mM DTT, 10 nmol substrate, and aRNase J (0.5 μg, 5 μmol) (+) or mock control (−) as indicated. Reactions were incubated at 50° C. for 80 min, unless otherwise indicated. aRNase J, aRNase J-StrepII homodimer purified by StrepTactin and Superdex 200 size exclusion chromatography from H. volcanii HS01-pJAM1406. Mock control, no enzyme. Substrates: 5′D and 3′D, TTCGGCGACTGATGTTGATTGGC (23 ntd) labeled at the 5′- and 3′-end with 6-FAM (carboxyfluorescein); 5′R and 3′R, UUCGGCGACUGAUGUUGAUUGGC (23 ntd) labeled at the 5′- and 3′-end with 6-FAM; 5′R-BL, CGAACUGCCUGGAAUCC*U*G*U*CGAACUGUAG labeled at the 5′-end with 6-FAM and internally labeled (BL, body labeled) with phosphorothioate (*)
(B) aRNase J nuclease activities compared to empty vector control. Reactions as in panel A with aRNasc J-StrepII homodimer purified from H. volcanii HS01-pJAM1406 compared to protein fractions similarly purified from H. volcanii HS01-pJAM202c (empty vector control, V).
(C) aRNase J requires Mg²⁺ or Mn²⁺ (not Zn²⁺) for activity. Reactions (10 μL): aRNaseJ (8.5 μmol) or mock control (−), 3 nmol substrate, 2 M NaCl, 50 mM HEPES (pH 7.5), 1 mM DTT and 5 mM divalent metal (MnCl₂, ZnCl₂ or MgCl₂) were incubated at 50° C. for 20 min (as indicated).
(D) Unlabeled oligonucleotides impact aRNase J nuclease activity. Unlabeled DNA (in cis, lanes 1-4) and RNA (in trans, lanes 9-12) reduce aRNase J hydrolysis of 5′D, while unlabeled RNA (in cis, lanes 5-8) but not DNA (in trans, lanes 13-16) reduce aRNase J hydrolysis of 5′R. Assay conditions as in panel A with 5 nmol substrate and unlabeled DNA and RNA at the 4-, 8- and 16-fold concentrations indicated.
(E) aRNase J enzyme kinetics. Reactions (10 μL): aRNaseJ (0.17 μg, 1.7 μmol), 2 M NaCl, 50 mM HEPES, pH 7.5, 1 mM DTT, 5 mM MnCl₂, 5 mM MgCl₂ and 3 nmol substrate. Reactions were incubated at 50° C. for 0, 5, 10, 15 and 20 min (as indicated). No hydrolysis was observed in enzyme minus reactions similarly incubated. ImageJ was used to quantify the rate of substrate hydrolysis and calculate k_{cat} and Vₘₐₓ based on experimental triplicates. Results were experimentally reproducible. Substrates: 5′D and 3′D, TTCGGCGACTGATGTTGATTGGC (23 ntd) labeled at the 5′- and 3′-end with 6-FAM (carboxyfluorescein); 5′R and 3′R, UUCGGCGACUGAUGUUGAUUGGC (23 ntd) labeled at the 5′- and 3′-end with 6-FAM; 5′R-BL, CGAACUGCCUGGAAUCC*U*G*U*CGAACUGUAG labeled at the 5′-end with 6-FAM and internally labeled (BL, body labeled) with phosphorothioate (*). Molecular mass standards: M_{RNA}, 5′ 6FAM-UUCGG and 5′ 6FAM-UUCGGCGACU with or without substrate; M_{DNA}, 5′ 6FAM-TTCGG and 5′ 6FAM-TTCGGCGACT. Unlabeled DNA and RNA: TTCGGCGACTGATGTTGATTGGC and UUCGGCGACUGAUGUUGAUUGGC.

FIG. 6 shows nuclease activities of RecJ3/4 (A), RecJ3/4-aRNase J (B) complexes, and the impact of RecJ3/4 on aRNase J activity (C).
(A) RecJ3/4 nuclease activities. (i) RecJ3/4 (+) activity compared to mock control (−). Reactions (10 μL): RecJ3/4 (6.7 μmol, 3.75 μg) or mock control, 2 M NaCl, 50 mM HEPES, pH 7.5, 1 mM DTT, 5 mM MnCl₂, 5 mM MgCl₂ and 3 nmol substrate incubated at 50° C. for 80 min. (ii) Metal dependence of RecJ3/4. Standard reaction was modified to selectively include the divalent cations MnCl₂, MgCl₂ and ZnCl₂ at 5 mM as indicated.
(B) RecJ3/4-aRNase J nuclease activities. (i) RecJ3/4-aRNase J activity (+) compared to mock control (−), empty vector (V), heat treatment (H), and EDTA treatment (E) as indicated. Standard reaction (10 μL): RecJ3/4-aRNase J (0.5 μg) or controls, 20 mM Tris-HCl (pH 7.5), 2 M NaCl, 5 mM MnCl₂, 5 mM MgCl₂, 1 mM DTT and 5-10 nmol substrate. Reactions were incubated at 50° C. for 80 min. RecJ3/4-RNaseJ, purified at 2:2:1 stoichiometry by TAP from H. volcanii HJ02-pJAM4252. Vector, protein fractions purified by TAP from H. volcanii HJ02-pJAM202c. H, RecJ3/4-aRNase J incubated at 99° C. for 20 min prior to assay. E, EDTA (10 mM) included in assay. (ii) Impact of excess unlabeled oligonucleotides on RecJ3/4-aRNase J activity. Unlabeled DNA or RNA included in assay at 1- to 8-fold (1×, 2×, 4× and 8×) concentration of substrate (5 nmol) as indicated. RecJ3/4-RNaseJ with no added unlabeled nucleotides (+) and TPEN (10 mM tetrakis-(2-pyridylmethyl)ethylenediamine) inhibited (−) included for comparison.
(C) Impact of RecJ3/4 on aRNase J activity. (i) SDS-PAGE gel of aRNase J (0.5 μg per lane) mixed with increasing concentrations of RecJ3/4 (0, 1, 2, and 4 μg, lanes 1-4) and (ii) corresponding nuclease activity assays with reactions (10 μL): 20 mM Tris-HCl (pH 7.5), 2 M NaCl, 5 mM MnCl₂, 5 mM MgCl₂, 1 mM DTT, 5-10 nmol substrate, and enzyme as in the SDS-PAGE gel.
   All results were found to be experimentally reproducible. Molecular standards, substrates and unlabeled oligonucleotides abbreviated as in FIG. 5.

FIG. 7 shows RecJ homolog domain architecture (A) and co-occurrence pattern of RNA exosome compared with RecJ3/4-aRNase J homologs among archaea (B). Domains include: DHH, DHH phosphoesterase superfamily IPR038763; DHHA1 (DHH associated domain 1) IPR003156; OB, nucleic acid binding OB-fold IPR012340 or RecJ OB domain IPR041122; S1, S1 RNA binding specific domain of the OB-fold superfamily IPR022967; Zf, zinc finger motif of IPR001305; IDR, intrinsically disordered region. EcRecJ (EC=Escherichia coli; P21893); Homo sapiens HsCdc45 (HS═Homo sapiens; 075419); PfHAN (Pf=Pyrococcus furiosus; Q8U3Q7); TkoHAN (Tko=Thermococcus kodakarensis; Q5JFJ4) and TkoGAN (Q5JGLO), with UniProt numbers in parenthesis. Clusters 1 and 2, two distinct clusters observed for RecJ homologs of IPR038763, IPR012340 and IPR022967 (met all three criteria) by sequence similarity network (SSN) analysis at an alignment score of 100. (B) Interpro (domain, family and/or superfamily) used to determine assignments included: Rrp41 (IPR011807), Rrp42 (IPR020869), Rrp4 (IPR023474), Csl4 (IPR039771), Nop5 (IPR000692), DnaG (IPR020607), RccJ/OB/S1 (IPR038763, IPR012340, and IPR022967); RNase J (IPR004613). Arcfu, Archaeoglobus fulgidus; Metsm, Methanobrevibacter smithii ATCC 35061; Metth, Methanothermobacter thermautotrophicus; Metst, Methanosphaera stadtmanae; Metmp, Methanococcus maripaludis S2; Metva, Methanococcus vannielii SB; Metae, Methanococcus aeolicus Nankai-3; Metja, Methanocaldococcus jannaschii; Metvu, Methanocaldococcus vulcanius M7; Metin, Methanocaldococcus infernus ME; Metka, Methanopyrus kandleri; Pyrae, Pyrobaculum aerophilum; Pyrar, Pyrobaculum arsenaticum DSM 13514; Pyris, Pyrobaculum islandicum DSM 4184; Thene, Thermoproteus neutrophilus V24Sta; Pyrca, Pyrobaculum calidifontis JCM 11548; Thete, Thermoproteus tenax; Calma, Caldivirga maquilingensis IC-167; Thepe, Thermofilum pendens Hrk 5; Censy, Cenarchaeum symbiosum; Nitma, Nitrosopumilus maritimus SCM1; Naneq, Nanoarchaeum equitans; Korcr, Candidatus Korarchaeum cryptofilum OPF8; Calte, Candidatus Caldiarchaeum subterraneum. ?, few examples restricted to environmental samples. Method used to present co-occurrence data adapted from reference (35).

FIG. 8 shows a model of H. volcanii RecJ3/4 and aRNase J nuclease activities in complex and subcomplex forms based on in vitro activity assays. RecJ3/4>>aRNase J indicates activities observed with 2-fold or more abundance of RecJ3/4 to aRNase J. Based on size exclusion chromotography, the RecJ3/4 subcomplex appears primarily in a tetramer of heterodimers configuration, while aRNase J alone forms a homodimer. Oligonucleotide substrates listed in grey boxes, abbreviated as in FIG. 5. aRNase J endoribonuclease activity was detected, while its *5′-3′ exonuclease activity is presumed based on analogy to other aRNase J enzymes.

FIG. 9 (mBio00852-23 S0004.pdf). Deletion of rnj (aRNase J) gene in H. volcanii H26carrying plasmid pJAM4253 (rnj+). Deletion of rnj (RNase J) gene in H. volcanii H26 carrying plasmid pJAM4253 (rnj+). PCR screening of the Δrnj deletion in H. volcanii H26 carrying plasmid pJAM4253 (rnj+). Lane 1-13, single colony strains screened. Lane 14, H26-pJAM4253 parent that was not subjected to homologous recombination. HJ06 strains with the Δrnj deletion are indicated by ●. Plasmid pJAM4253 could not be cured from the HJ06 Δrnj mutant strains. When using the same Δrnj deletion plasmid (pJAM4261), the Δrnj mutation could not be generated in the H26 strain alone or in H26 strains carrying the empty vector (pJAM202c).

FIG. 10 (mBio00852-23 S0005.pdf). Reducing SDS-PAGE gels of RecJ3, RecJ4, RNase J, and Cdc48a proteins purified from recombinant E. coli (A) and H. volcanii (B). The affinity tagged proteins were expressed and purified by StrepTactin (RNase J-StrepII and RecJ4-StrepII) or HisTrap (His-RecJ3, His-RecJ4 and His-Cdc48a) chromatography from the host strains as indicated. For purification of His-RecJ4 from E. coli, the flowthrough fractions from the His-Trap column were found to contain the soluble protein. All other proteins were found to bind the affinity column. For purification of RecJ4-StrepII from H. volcanii, the buffers were supplemented with YATP as indicated and detailed in methods. Vector, indicates the samples similarly purified from the H. volcanii host strain carrying the pJAM202c empty vector control. Purified proteins were separated by reducing SDS-PAGE and stained with Coomassie blue, SyproRuby or BioSafe Coomassie blue. Black bars on the right side of the gels in the panel B indicate regions where the gel slices were excised and analyzed by LC-MS/MS. Proteins indicated on right were identified by LC-MS/MS analysis based on a high score (SEQUEST HT or Normalized Total Spectra score) and FDR <0.01%, protein threshold >99%, 2 peptide minimum.

FIG. 11 (mBio00852-23 S0006.pdf). Immunoblotting analysis reveals multiple bands of Cdc48a consistent with its Ubl-modification. H. volcanii cells (OD600 of 0.08 units per lane) were lysed in reducing SDS-PAGE sample buffer by boiling 5-10 min. After cooling, the samples were separated by 10% reducing SDS-PAGE and analyzed by immunoblotting with anti-VCP antibody (AbCam product no. ab138298). Lane 1, H26-pJAM1400 (Cdc48a-StrepII ectopically expressed). Lane 2, NN2 (Δcdc48b). Lane 3, NN3 (Δcdc48c). Lane 4, H1999 (Δcdc48a).

FIG. 12 (mBio00852-23 S0007.pdf). Examples of genome neighborhoods of archaeal RecJ3, RecJ4, RNase J and Cdc48a gene homologs. Orange, Cdc48a, RecJ3/4 and RNase J used as ‘targets’; yellow and blue, homologs in synteny with ‘targets’ in at least 70 other archaeal genomes; grey, no distinct Pfam or InterPro classification, thus, limiting estimation of co-occurrence by GNN analysis; dark and light green, H. volcanii PRC, Nob1 and Tif5B gene homologs. See Table S1 and Dataset S2B for details and abbreviations (Jia et al., infra).

FIG. 13A (mBio00852-23 S0008.pdf). Cdc48a, RecJ3, RecJ4, and aRNase J homologs analyzed by 3D-structural modeling, protein domain analysis, and multiple amino acid sequence alignment. Comparison of Ub/Ubl-modification sites of H. volcanii (Hvo) Cdc48a to human p97. Cryo-EM structure of ATPγS bound human p97 (PDB: 5FTN, left panels) and Phyre2-generated 3D-homology model of H. volcanii Cdc48a (right panels) displayed in hexameric configuration. Ub/Ubl-modification sites are displayed in red and yellow spheres with lysine position numbers indicated. Cis, indicates the interface that engages in the unraveling of protein substrates. Trans, designates the surface that harbors a portal where protein substrates exit the complex in an unfolded state.

FIG. 13B Comparison of H. volcanii RecJ3 and RecJ4 to Thermus thermophiles (Tth) RecJexonuclease. H. volcanii (Hvo) Hvo-RecJ3 (HVO_1018) and Hvo-RecJ4 (HVO_2889) 3D-structural models were generated by Phyre2-based homology modeling and compared to the X-ray crystal structure of Tth-RecJ (PDB: 1IR6, chain A). Table inset: conserved active site residues that may coordinate the catalytic Mn2+ ion (red) and residues suggested to bind a second metal ion in the presence of ssDNA or nucleotide (brown). Residues are also highlightedon the ribbon diagrams.

FIG. 13C Multiple amino acid sequence alignment of bacterial and archaeal aRNase J family proteins. UniProtKB numbers (left): Bacillus subtilis Bsu-RNase J1/2 (BsRnj1/2, Q45493/031760), Mycobacterium tuberculosis Mtu-RNase J (P9WGZ9), Pyroccous absysii Pab-aRNase J (Q9V076), Methanocaldococcus jannaschii Mja-aRNase J3 (Q58271), Thermococcus kodakarensis Tko-aRNase J (Q5JH57), and Haloferax volcanii Hvo-aRNase J (D4GW49). ●, conserved active site residues that coordinate two divalent metal ions (Zn2+ or Mg2+) for catalysis. The C-terminal tail common to the bacterial RNase J is not conserved in the archaeal enzymes.

FIG. 13D RNase J family protein domain architecture. Domains/families (InterPro): Rnj, RNase J family (IPR004613); TM, transmembrane domains; FtsK α, P-loop and γ domains (IPR041027, IPR002543 and IPR018541); PBP, penicillin-binding protein domains (IPR005311 and IPR036138), DnaK chaperone family (IPR012725), MFS, major facilitator superfamily (IPR011701); UppP/BacA, undecaprenyl-diphosphatase (IPR003824); IDR, intrinsically disordered region; CC, coiled coil. RNase J homologs (Uniprot): Ab, Actinobacteria (A0A537ZKW0); Fb, Fusobacterium (G6C271); Pb, Parcubacteria (A0A0G1TWX8); Nb, Nomurabacteria (A0A3D5Y638); Pm, Planctomycetales (A0A1V4QR24); Cb Corynebacterium bovis (A0A426Q1M6); Mp, Mycoplasma ovipneumoniae (A0A449AXP7); Hp, Helicobacter pylori (B9XZG7); Bs-1/2, Bacillus subtilis (Q45493/031760); Hv, Haloferax volcanii (D4GW49).

FIG. 13E 3D-structural comparison of Haloferax volcanii Hvo-aRNase J and Methanolobus psychrophilus Mpy-aRNase J. Surface representation colored according to Coulombic surface charge. The molecules on the bottom (‘back’) are rotated 180° compared to the top (‘front’) to provide a more complete visualization of the surface charge. Hvo-aRNase J, 3D-structural model Alpha-fold AF-D4GW49-F1. Mpy-aRNase J, X-ray crystal structure PDB: 6LLB of enzyme with S247A variant in complex with 6 nt RNA, the latter in stick diagram.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “some embodiment,” “certain embodiment,” means that a particular feature, structure or characteristic described in connection with the embodiment is included in at least one embodiment of the present invention. Thus, appearances of the phrases “in one embodiment,” “in some embodiment,” or “certain embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “archaea” refers to a domain of single-celled organisms. Archaea were initially classified as bacteria and known as archaebacteria, but this term has fallen out of use. Like bacteria, archaea are single-celled microorganisms lacking cell nuclei and organelles. In addition, like bacteria, they have a cell wall, flagella, and single, circular DNA, and reproduce by fission (in which a parent cell splits into two daughter cells). However, there are some differences between archaea and bacteria. For example, bacterial cell walls contain peptidoglycan and lipopolysaccharide, but archaeal cell walls do not have peptidoglycan and contain pseudopeptidoglycan, polysaccharides, glycoproteins, or protein-based cell walls. In addition, bacterial cell membrane is ester-linked lipids whereas archaeal cell membrane is ether-linked lipids; and archaea perform modified form of glycolysis and citric acid cycle, and they are found in extremely harsh environments such as hot springs, marshlands, deep sea vents, and the gut of humans and ruminants.

However, although archaea are prokaryotes like bacteria, the molecular systems of archaea are more similar to those of eukaryotes than to bacteria. For instance, the proteins involved in DNA replication, transcription, and translation are quite similar to the eukaryotic ones. Thus, these microorganisms are valuable model systems for molecular biology study for eukaryotic system.

As used herein, “aRNase J” refers to archaea RNase J. RNase J is a prokaryotic ribonuclease present in many bacteria and known to play a critical role in both RNA processing and degradation. Bacterial RNase J and its homologs are unique among all known ribonucleases in that it has both endoribonucleolytic and 5′-3′ exoribonucleolytic activities, with some exception such as B. subtilis RNase J1, which is suggested to function in vivo only as 5′-3′ exoribonucleascs, as the endonucleolytic mechanism would require the homodimer to dissociate and the β-CASP and β-lactamase domains to separate in order for the RNA substrate to lie across the active site and be cleaved.

The term “nucleotide” as used herein refers to a subunit of a nucleic acid (whether DNA or RNA or an analogue thereof) which may include, but is not limited to, a phosphate group, a 5-carbon sugar group and a nitrogen containing base, as well as analogs of such sub-units. Other groups (e.g., protecting groups) can be attached to the sugar group and nitrogen containing base group. It will be appreciated that, as used herein, the terms “nucleotide” and “nucleoside” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”).

The terms “nucleic acid strand,” “nucleotide sequence,” and “nucleic acid sequence” as used herein refer to any polyribonucleotide or polydeoxyribonucleotide that may be unmodified RNA or DNA or modified RNA or DNA. Thus, for instance, nucleic acid as used herein refers to, among others, single and double-stranded DNA, DNA that is a mixture of single and double-stranded regions, single and double-stranded RNA, and RNA that is mixture of single and double-stranded regions, hybrid molecules comprising DNA and RNA that may be single-stranded or, more typically, double-stranded or a mixture of single and double-stranded regions. In one aspect, the nucleic acid can be ssDNA. In another aspect, the nucleic acid can be RNA. Suitable nucleic acid molecules are DNA, including genomic DNA or cDNA. Other suitable nucleic acid molecules are RNA, including mRNA, rRNA, and tRNA.

The term “sequence identity” or “identity,” as used herein in the context of two polynucleotides or polypeptides, refers to the residues in the sequences of the two molecules that are the same when aligned for maximum correspondence over a specified comparison window. As used herein, the term “percentage of sequence identity” or “% sequence identity” refers to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

The term “vector” as used herein refers to a nucleic acid molecule capable of transporting another nucleic acid to which it has been linked. It is usually a DNA molecule that is used as a vehicle to carry a particular foreign nucleic acid sequence, usually DNA, into a host/recipient cell where it can be replicated and/or expressed. The vector typically includes features to facilitate the manipulation of DNA as well as a genetic marker for their selective recognition. The most common vectors are DNA plasmids, viruses and artificial chromosomes. Sometimes, “plasmid” and “vector” may be used interchangeably as the plasmid is the most commonly used form of vector. The vectors can be autonomously replicated in a host cell (episomal vectors), or may be integrated into the genome of a host cell, and replicated along with the host genome (non-episomal mammalian vectors). Integrating vectors typically contain at least one sequence homologous to the bacterial chromosome that allows for recombination to occur between homologous DNA in the vector and the bacterial chromosome. Integrating vectors may also comprise bacteriophage or transposon sequences.

The term “plasmid” as used herein refers to a double-stranded, covalently closed, circular DNA into which additional DNA segments can be ligated and which can be isolated from bacterial cells. Plasmid exists in its bacterial hosts as extrachromosomal pieces of DNA that vary in size from 1 kb to >200 kb. Most of the plasmids used in molecular cloning have a multiple cloning site (MCS), also called a polylinker, which is a short segment of DNA which contains various restriction sites a standard feature of engineered plasmids for the insertion of the foreign DNA. In addition, the plasmid should have an origin of replication (ori) site-usually bacterial origin where DNA replication is initiated, marker genes-antibiotics resistance gene for selection and/or screening with antibiotics, and promoters-usually viral origin for gene expression. It should be small in size so that it can be easily delivered into the host cell. Plasmids do not generally replicate in the host mammalian cells. By performing a process of DNA transfection or transformation, a plasmid which contains a gene of interest is efficiently delivered to the cells of interest. Numerous plasmid vectors are commercially available, and the modification thereof for specific cloning strategies is well known to the skilled person in the field.

As used herein, a “promoter” is defined as a regulatory DNA sequence that is generally located upstream of a gene and capable of binding RNA polymerase to initiate transcription. In some embodiments, the plasmid may comprise more than one RNA polymerase II (pol II) promoters and/or RNA polymerase III (pol III) promoters. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process. A well known example of an inducible promoter is the lac operon. In the lac operon system, a promoter for lactose operon has an operator sequence between the promoter and transcription initiation site. The operator sequence is the site where transcription repressor binds, which is removed from the operator site when an inducer, allolactose or isopropyl β-d-1-thiogalactopyranoside (IPTG) binds to the repressor. Another example is arabinose or tryptophan-induced promoter.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. The terms “protein” and “polypeptide” as used herein refer to both large polypeptides and small peptides. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

### Overview

Nucleases are strictly regulated and often localized in the cell to avoid the uncontrolled degradation of DNA and RNA. Here, a new type of nuclease complex, composed of RecJ3, RecJ4, and aRNase J, was identified through its ATP-dependent association with the ubiquitin-like SAMP1 and AAA-ATPase Cdc48a. The complex was discovered in Haloferax volcanii, an archaeon lacking an RNA exosome. Genetic analysis revealed aRNase J to be essential and RecJ3, RecJ4, and Cdc48a to function in the recovery from DNA damage including genotoxic agents that generate double-strand breaks. The RecJ3:RecJ4:aRNase J complex (isolated in 2:2:1 stoichiometry) functioned primarily as a 3′-5′ exonuclease in hydrolyzing RNA and ssDNA, with the mechanism non-processive for ssDNA. aRNase J could also be purified as a homodimer that catalyzed endoribonuclease activity and, thus, was not restricted to the 5′-3′ exonuclease activity typical of aRNase J homologs. Moreover, RecJ3 and RecJ4 could be purified as a 560-kDa subcomplex in equimolar subunit ratio with nuclease activities mirroring the full RecJ3/4-aRNase J complex. These findings prompted reconstitution assays that suggested RecJ3/4 could suppress, alter, and/or outcompete the nuclease activities of aRNase J. Based on the phenotypic results, this control mechanism of aRNase J by RecJ3/4 is not necessary for cell growth but instead appears important for DNA repair.

### RecJ3, RecJ4, and aRNase J Complex

As described herein, a new type of nuclease complex, composed of RecJ3, RecJ4, and aRNase J, is identified in H. volcanii. The complex functions primarily as a 3′-5′ exonuclease on RNA and ssDNA substrates, with the mechanism non-processive for ssDNA. Related subcomplexes could be isolated including: (i) an aRNase J homodimer that catalyzed endonuclease activity with preference for RNA over ssDNA and (ii) a 560-kDa subcomplex of RecJ3 and RecJ4 in equimolar ratio with 3′-5′ exonuclease activities paralleling that of the full RecJ3/4-aRNase J complex.

Nuclease activities were found to shift primarily to 3′-5′ exonuclease when the ratio of RecJ3/4 to aRNase J was increased suggesting aRNase J (endonuclease and 5′-3′ exonuclease) activity was sequestered, altered, or outcompeted for nucleotide substrate by RecJ3/4 (FIG. 8). The nucleases were found to require Mg²⁺ or Mn²⁺ (vs Zn²⁺), and were active in high concentrations of salt (2 M NaCl), which may reflect the salt-in strategy used by H. volcanii to maintain homeostasis in hypersaline ecosystems. Similar salt requirements are observed for many other H. volcanii enzymes, e.g., proteasomes, inorganic pyrophosphatase (73, 74), but surprisingly not H. volcanii RNase R or RNase Z which are inactive in concentrations of >0.2 M salt (75-77).

### Ubl, Cdc45a and RecJ3, RecJ4, aRNase J Complex

The RecJ3/4-aRNase J complex was discovered based on its ATP-dependent association with Ubl SAMP1 and Cdc48a. Cdc48a is the proposed candidate that facilitates the Ubl binding of the RecJ3/4-aRNase J complex. This hypothesis is based on the relationship of Cdc48a to ATPases that are biologically linked to Ubl-/Ub-modification proteasome systems (55, 56, 68, 78-83). Moreover, Cdc48-type ATPases are found to alter protein-protein and/or protein-nucleic acid interactions through ATP-dependent conformational shifts (56, 82-84).

Cdc48a is found Ubl modified at K210, K216, and K723. These residues are conserved in diverse archaea and analogous to PTM hot spots of the related human p97 (85, 86) (FIG. 13A). Based on 3D-structural comparison (68, 87), Cdc48a K210 and K216 are residues predicted to be located at an intersubunit interface that may engage in substrate protein unraveling, while K723 appears positioned where the substrate protein, once unfolded, would pass through the ATPase pore. Thus, the observed Ubl modifications of Cdc48a are in positions that may impact biological function. Other members, of the H. volcanii AAA ATPase network, are also post-translationally modified (51, 88). aRNase J is found essential in H. volcanii. Central roles for RNase J (IPR004613) family proteins have been observed in other domains of life. In Bacillus subtilis, RNase J1 mutations majorly impact cell morphology, sporulation, competence (89), and the resolution of stalled transcription complexes (24). Staphylococcus aureus RNase J mutations cause global defects in RNA maturation and degradation (90). In plant chloroplasts, RNase J null mutants are embryo-lethal (91). The findings here that H. volcanii (Hvo-) aRNase J is essential suggests this enzyme has a key role in RNA turnover and is in contrast to the Thermococcus barophilus Tba-aRNase J which is non-essential (13). One notable difference, which may explain these findings, is that T. barophilus encodes an RNA exosome that is central to RNA metabolism, whereas H. volcanii does not.

RecJ3, RecJ4, and Cdc48a are shown to be important for the recovery of H. volcanii from DNA-damaging agents. Correlating these phenotypes with the in vitro activity assays suggests the RecJ3/4 3′-5′ exonuclease activities and/or the constraint these proteins have on Hvo-aRNase J endonuclease activity may be associated with DNA repair. Mounting evidence reveals RNA and RNA-processing enzymes are important in DNA recombination and repair (5, 6). For example, DNA:RNA hybrids at DNA break sites facilitate homologous recombination and include the recruitment of RNases such as RNase H2 and RNA exosomes in eukaryotes (6).

RecJ3 is found tightly associated with RecJ4 in H. volcanii, suggesting the annotation of HVO_1018 (RecJ3) as a HAN may need to be broadened. RecJ3 and Hef are observed to be among the top 200 proteins that co-purify with Ubl SAMP3 in H. volcanii (54). Other proteins that co-purify in the Ubl network (54) include Hvo-aRNase J, Cdc48a, and other homologs of DNA repair, recombination, and nucleotide hydrolysis such as aCPSF1 (HVO_0874), CRISPR Cas7/8b (HVO_A0207/HVO_A0206), RadA (HVO_0104), enolase (HVO_2774), Tif5B (HVO_1963), UvrA (HVO_0393), NthB (HVO_0878), AP endonuclease (HVO_2322), and MutS/L (HVO_0552/HVO_0551). While these findings provide evidence that RecJ3 and Hef are members of a Ubl interactome (54), this level of selection from a theoretical proteome of 3,996 proteins (Uniref UP000008243) is not conclusive evidence that RecJ3 and Hef form a complex in H. volcanii.

### RecJ4 as Scaffold for RecJ3 and aRNaseJ Binding

RecJ4 functions as an apparent scaffold for the association of Hvo-aRNase J with RecJ3. Consistent with this role, RecJ4 lacks conserved active site residues (FIG. 13B) and captures Hvo-aRNase J and RecJ3 when used in affinity-tagged pull-down assays. Furthermore, a recJ4 mutation disrupts the association of RecJ3 with Hvo-aRNase J. RecJ4 has an extensive IDR that spans residues 100-235 sandwiched between S1 and OB-fold domains including an acidic patch at positions 185-227 (FIG. 7A). This IDR is related in primary sequence to RecJ homologs that cluster to arCOG00429. RecJ3 also has an IDR; however, this region spans only 22 residues. As IDRs often provide binding sites for protein partners (92), this may explain why RecJ4 is required for Hvo-aRNase J to co-purify with RecJ3.

### Other Subunits Associating with RNase J Family

Protein partners of RNase J (IPR004613) family proteins are widespread and diverse. In bacteria, RNase J assembles into multi-subunit RNA degradosomes and associates with translating ribosomes, chaperones, and membranes (93-98). These types of interactions appear to be promoted by N- and C-terminal extensions and/or fusions to other types of domains (e.g., FtsK, DnaK, MFS, and UppP/BacA) (FIGS. 13C and 5D). Surprisingly, aRNase J proteins are missing these extensions and extra domains, yet still bind protein partners as exemplified by Hvo-aRNase J binding to RecJ3/4 (this study) as well as the association of Pyrococcus abyssi Pab-aRNase J with a Ski2-like helicase linked to the RNA exosome (13). Whether these interactions are regulated by PTMs remains to be determined. RNase J is covalently attached to Pup (a small, intrinsically disordered prokaryotic Ubl) in mycobacteria, while aRNase J (along with its protein partners) is found lysine acetylated in haloarchaea (99, 100).

### Ribonuclease Activity of aRNas J

Hvo-aRNase J catalyzes endoribonuclease activity and, thus, is not restricted to the 5′-3′ exonuclease activity typical of other aRNase J homologs. Hvo-aRNase J hydrolyzed all oligonucleotide substrates examined in an Mg²⁺/Mn²⁺ (vs Zn²⁺)-dependent manner and displayed a preference for RNA vs ssDNA based on k_{cat} and Vₘₐₓ values determined at saturating substrate (>1,500-fold molar excess). This type of profile is more typical of bacterial RNase J enzymes which can function as 5′-3′ exo- and/or endoribonucleases (7, 18-23).

Several possibilities may explain why Hvo-aRNase J has endonuclease activity (and is not restricted to 5′-3′ exonuclease activity). (i) Hvo-aRNase J shares only 33%-52% amino acid sequence identity with the characterized aRNase J enzymes. (ii) Hvo-aRNase J forms a homodimer, while the other aRNase J characterized at this level (i.e., Mpy-aRNase J) forms a homotetramer with a dimer-dimer interface apparently immobilizing the RNA-binding channel (15). The monomeric unit of Hvo-aRNase J is predicted to have an extreme acidic shell (FIG. 13E), which may promote this different subunit configuration. In further support of this possibility, bacterial RNase J enzymes that are endonucleases use entirely different subunit interfaces than Mpy-aRNase J to associate in dimers and tetramers (23). (iii) Hvo-aRNase J functions in 2 M NaCl with catalytic Mg²⁺ or Mn²⁺ ions, thus, contrasting with other aRNase J enzymes which use Zn²⁺. (iv) Hvo-aRNase J is purified from H. volcanii (vs recombinant E. coli) adding the possibility that PTMs alter its activity (e.g., lysine residues at positions 112, 120, 191, 263, and 278 are found acetylated) (100). (v) The 3D modeling of Hvo-aRNase J suggests conserved active site residues (e.g., Ser250, His391, and His83) are located on larger loops when compared to Mpy-aRNase J (Alpha-fold AF-D4GW49-F1 vs PDB: 6LLB) (15), which could provide greater flexibility in the nuclease mechanism. (vi) Longer or highly structured RNA substrates may be required for Hvo-aRNase J to strictly favor 5′-3′ exoribonucleolytic activity.

The RecJ3/4-aRNAse J complex may provide a hub for RNA degradation in H. volcanii, as this archacon lacks an RNA exosome and polyadenylated RNA (101). Many archaca harbor aRNase J enzymes that appear to engage with RNA exosomes: RNA-degrading machines with 3′-5′ exoribonuclease and polyadenylation activities (13, 101). The polyadenylated poly(A) tails at the 3′-end of RNA can provide a toehold for 3′-5′ exonucleases to degrade the RNA (102). At one time, RNase R was hypothesized to be the sole exoribonuclease of H. volcanii, but this enzyme is not active in “high-salt” conditions (76, 77) that mimic the cytosol of this organism (103). Here, RecJ3/4 was found to mediate 3′-5′ exoribonuclease activity and to associate with aRNase J in high-salt conditions. These types of activities and protein-protein interactions may provide functional replacements for the aRNase J and RNA exosome associations observed in other archaea.

### Summary of Results

Overall, RecJ3/4-aRNase J is a newly identified nuclease complex. This complex appears conserved in halophilic and methanogenic archaea that do not encode an RNA exosome and is strikingly absent from the archaeal TACK group, members of which have RNA exosomes. The RecJ3/4-aRNase J complex associates with Ubl SAMP1 (likely via Cdc48a). This binding is speculated to localize and/or coordinate the degradation of nucleic acids in the cell, including DNA repair based on the ΔrecJ3, ΔrecJ4, and Δcdc48a mutant phenotypes. Binding to Hvo-RecJ3/4 reduces the endonuclease activity detected for Hvo-aRNase J, and renders the overall function of the complex as a 3′-5′ exonuclease. Interestingly, ssDNA appears to stimulate the hydrolysis of the 5′-end-labeled RNA substrate by the RecJ3/4-aRNase J complex, consistent with the possibility that this nuclease complex could play a role in resolving DNA:RNA hybrids or R-loops that actively form at DNA double-strand breaks (104). Whether the RecJ3/4-bound Hvo-aRNase J is targeted for degradation by the Ubl-proteasome system remains to be determined. The Archaeal Proteomic Project (ArcPP), an assembly of proteomic data sets derived from H. volcanii grown in different labs and environmental conditions (105), reveals Hvo-aRNase J along with RecJ3, RecJ4, and Cdc48a that are detected in all conditions examined. By contrast, the Ubl SAMPs are identified only in a subset of the proteomic data sets. While not quantitative, these results point toward environmental cues signaling shifts in the protein abundance of Ubl SAMP and not the subunits of the nuclease complex. Thus, the Ubls (via Cdc48a) may regulate the protein-protein interactions of the RecJ3/4-aRNase J complex instead of targeting its subunits for degradation.

### Examples of Embodiments

Based on the results of the experiments described above, and in the Examples section below, a novel nuclease complex system is provided in this disclosure, which shifts RNase activity from endonuclease and 5′-3′ exonuclease activity to 3′-5′ exonuclease activity, for controlling direction of nucleotide degradation in a sample comprising a single-stranded DNA or RNA. In one embodiment, disclosed is a nuclease complex system comprising aRNase J and RecJ3/4 isolated from Haloferax volcanii.

The ratio of RecJ3/4 heterodimer and aRNase J homodimer can be changed from 0:0:1 to 2:2:1, wherein RecJ3 and RecJ4 are of an equimolar ratio, and in particular the ratio of RecJ3/4 heterodimer and aRNase J homodimer can be 0:0:1, 2:2:1 or 1:1:2. For example, (i) at the ratio of 0:0:1 of RecJ3, RecJ4, and aRNase J, aRNase J has activity as endonuclease and 5′-3′ exonuclease; (ii) at a low ratio, the complex has more activity as endonuclease and 5′-3′ exonuclease than 3′-5′ exonuclease, (iii) at a high ratio, the complex has more activity as 3′-5′ exonuclease than endonuclease and 5′-3′ exonuclease; and (iv) at the ratio of 2:2:1, the complex has 3′-5′ exonuclease activity.

In some embodiments, the RecJ3, RecJ4 and aRNase J comprise an amino acid sequence with at least 90%, preferably at least 91%, at least 92%, at least 93%, at least 94%, at least 95%, at least 96%, at least 97%, at least 98%, at least 99% identify to the amino acid sequence as set forth in SEQ ID NO: 1, 3 and 5 respectively, or is encoded by a nucleotide sequence with at least 90%, preferably at least 91%, at least 92%, at least 93%, at least 94%, at least 95%, at least 96%, at least 97%, at least 98%, at least 99% homology to the coding nucleotide sequence as set forth in SEQ ID NO:2, 4 and 6, respectively.

In another embodiment, disclosed is an endo- to exonuclease activity titration kit of a nuclease complex system for in vitro use, comprising aRNase J and RecJ3/4. The kit further comprises concentrated aRNase J reaction buffer and optionally RecJ3/4 reaction buffer described in EXAMPLES, e.g., pH range between 7.2-7.7, Mg²⁺, Mn²⁺, NaCl, etc. reaction stop solution comprising EDTA and/or TPEN, and/or exonuclease activity titration substrates, RNA or ssDNA, labeled with 6-carboxyfluorescein (6-FAM) at its 3′-end or 5′ end, and RNAs labeled internally with phosphorothioate and at 3′ end with 6-FAM.

Further, RecJ3, RecJ4 and/or aRNase J can have a tag at its C-terminal or N-terminal for protein purification, wherein the tag can be selected from hexahistidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, etc.

In further embodiments, nucleic acid strands encoding His-RecJ3, RecJ4-StrepII, and/or aRNase J-StrepII proteins, recombinant vectors or plasmids for replication and/or protein expression, comprising those nucleic acid strands, in particular plasmids pJAM1406 (+rnj-strepII), pJAM4251 (+his6-recJ3), pJAM4252 (+his6-recJ3 and +rnj-strepII), and pJAM1405 (+recj4-strepII) are provided. Exemplary archaea strains comprising the nucleic acid strands or plasmids mentioned above, in particular the strains of H. volcanii including HJ05 (T₇-PtnaA-rnj), HJ02 (ΔrecJ3 pitA_{Nph}), HJ07 (ΔrecJ3 pitA_{Nph} Δrnj), HJ04 (ΔrecJ4 pitA_{Nph}) or HS01 (ΔrecJ3) are also provided.

In addition, purification methods of aRNase J, RecJ3, RecJ4, RecJ3/4 subcomplex, and RecJ3/4-aRNaseJ complex using HisTrap and/or Strep-Tactin affinity chromatography and size exclusion/gel filtration chromatography are also provided.

Further, an in vivo endo- and exonuclease system comprising aRNase J homodimer and RecJ3/4 heterodimers is provided, comprising a recombinant expression vector comprising a nucleic acid strand encoding RecJ3 along with a recombinant expression vector comprising a nucleic acid strand encoding RecJ4, or a recombinant expression vector comprising a nucleic acid strand encoding both RecJ3 and RecJ4. A promoter can be positioned upstream of the nucleic acid strand encoding either of RecJ3 or RecJ4, or both RccJ3 and RecJ4, and the promoter can be an inducible promoter regulated by tryptophan, IPTG, or arabinose. The system also comprises another recombinant expression vector comprising a nucleic acid strand encoding aRNase J, wherein it has another promoter different from said promoter positioned upstream of the nucleic acid strand encoding aRNase J, and it can be constitutive or inducible promoter.; Instead of comprising three or two vectors or plasmids, the system can comprise one recombinant expression vector comprising a nucleic acid strand encoding all three proteins of RecJ3, RecJ4 and aRNascJ, wherein a promoter can be positioned upstream of the nucleic acid strand encoding both RecJ3 and RecJ4 and another promoter different from said promoter can be positioned upstream of the nucleic acid strand encoding aRNaseJ. In addition, cells recombinantly engineered to express heterologous nucleic acid strands encoding RecJ3, RecJ4 and/or aRNase J, or a combination thereof can be provided.

Although RecJ3, RecJ4 and/or aRNase J can be isolated from archaea that do not encode a canonical RNA exosome, for example, Euryarchaeota comprising thermococcales, methanosarcinales, methanomicrobiales, haloarchaea and certain methanogens, Haloferax volcanii is preferred.

In other embodiments, provided is a recombinant expression vector comprising the coding sequence of the RecJ3, RecJ4 and/or aRNase J from Haloferax volcanii, or a host cell comprising the recombinant expression vector.

By conventional recombinant DNA technique (Science, 1984; 224:1431), the coding sequence of RecJ3, RecJ4 and aRNase J may be used to express or produce the recombinant protein. The methods well known to those skilled in the art can be used to construct an expression vector containing the coding sequence of the RecJ3, RecJ4 and/or aRNaseJ or active fragments thereof and the and appropriate transcriptional/translational control signals. These methods include in vitro recombinant DNA technique, DNA synthesis technique, in vivo recombination technique and the like. The DNA sequence can be operably linked to an appropriate promoter in the expression vector to direct mRNA synthesis. The expression vector further includes a ribosome binding site for translation initiation and a transcription terminator.

In addition, the expression vector may comprise one or more selectable marker genes to provide a phenotypic trait for selection of transformed host cells, such as hygromycin resistance gene for hygromycin-screening process, dihydrofolate reductase, neomycin resistance, and green fluorescent protein (GFP) for eukaryotic cell culture, or tetracycline or ampicillin resistance for E. coli.

The host cell can be a prokaryotic cell, such as a bacterial cell; or a lower eukaryotic cell, such as a yeast cell. The representative examples include: E. coli, Streptomyces spp., Agrobacterium spp. (such as A. tumefaciens and A. rhizogenes); fungal cells, such as yeast and the like. In the present invention, it is preferable to use Agrobacterium (e.g., A. tumefaciens and A. rhizogenes) as a host cell. Those ordinarily skilled in the art all know how to choose the appropriate vector, promoter, enhancer, and host cell.

In the above method, the recombinant polypeptide can be expressed intracellularly or on the membrane or secreted outside of the cell. If desired, the recombinant protein can be isolated and purified by various isolation methods according to its physical, chemical, and other properties. These methods are well known to those skilled in the art. Examples of such methods include, but are not limited to: conventional renaturation treatment, treatment with protein precipitants (salting out method), centrifugation, osmotic lysis, ultrasonic treatment, ultracentrifugation, molecular sieve chromatography (gel filtration), adsorption chromatography, ion exchange chromatography, high performance liquid chromatography (HPLC) and other various liquid chromatographic techniques and the combination thereof.

### EXAMPLES

### Example 1. Materials and Methods

The information in the examples below is further supported in Jia et al, mBio. 2023 Aug. 31; 14(4):e0085223, which is cited herein by reference in its entirety including the supplemental information (referred to herein as “Jia et al.”).

### 1.0 Materials.

Biochemicals were from Sigma-Aldrich (St. Louis, MO, USA) or Alfa Aesar by ThermoFisher Scientific (Tewksbury, MA, USA). Other organic and inorganic analytical grade chemicals were from Fisher Scientific (Atlanta, GA, USA). Restriction endonucleases, T4 DNA ligase and Phusion polymerase were from New England Biolabs (Ipswich, MA, USA). Taq and Pfu DNA polymerases were from Bioline (Taunton, MA, USA) and Stratagene (La Jolla, CA, USA), respectively. Desalted oligonucleotides were from Integrated DNA Technologies (Coralville, IA, USA) and Eurofins Genomics (Louisville, KY, USA). DNA sequencing was by Eton Bioscience Inc. (Research Triangle Park, NC, USA). Proteomics was performed at UF Interdisciplinary Center for Biotechnology Research (ICBR, Gainesville, FL, USA). Strep-Tactin Superflow Plus Resin (U.S. Cat. No. 1057978) was from Qiagen Inc. (Germantown, MD, USA). HisTrap HP 5 ml columns (GE Healthcare, Cat. No. 29-0510-215) were from Sigma-Aldrich. Cellulose acetate membranes, 0.2 μm, for protein filtration were from Thermo Scientific (Waltham, MA, USA). Phleomycin (Cat. No. ant-ph-1) was from InvivoGen (San Diego, CA, USA). Strains and media.

Strains, plasmids and primers used in this study are summarized in Table S1 (Jia et al.). E. coli GM2163 was used for replication of plasmid DNA prior to transformation into H. volcanii strains according to standard methods (106). E. coli strains were grown at 37° C. in Luria-Bertani (LB) medium (unless indicated as 25° C.). H. volcanii strains were grown at 42° C. in ATCC 974 Halobacterium medium, Casamino acids (CA) plus medium, Hv-YPC (yeast extract, peptone, and CA) medium, and Hv-Min medium (0.1% lactic acid, 0.09% succinic acid, and 0.01% glycerol served as carbon source) as described in the Halohandbook (105). Hv-Min was supplemented with 50 μg/mL uracil from a 50 mg/mL stock dissolved in DMSO (Hv-Min-Ura+). Cells were grown in liquid cultures with rotary shaking at 200 rpm and on solid medium. Media were supplemented per liter with ampicillin (Amp, 100 mg), kanamycin (Km, 50 mg), chloramphenicol (Cm, 30 mg), novobiocin (Nv, 0.2-0.3 mg), 5-fluoroorotic acid (5-FOA, 50 mg) and/or uracil (50 mg) as needed, unless otherwise indicated. Growth was monitored by measuring optical density at 600 nm (OD₆₀₀), where 1 OD₆₀₀ unit equals approximately 1× 109 colony forming units (CFU)/mL. Once constructed and verified, all H. volcanii strains were stored at −80° C. in 20% v/v glycerol stocks. To culture, the H. volcanii strains were streaked from −80° C. onto ATCC974 plates and incubated for 4-5 days at 42° C. for single colony. Media were supplemented with Nv for H. volcanii strains carrying the pJAM plasmids.

1.1. Conditional Depletion and Mutagenesis of rnj (aRNase J) in H. volcanii.

To analyze the role of rnj (aRNase J) in H. volcanii, two approaches were employed. First, the pyrE2-based pop-in pop-out method (107) was used to target the rnj gene for deletion by homologous replacement from the H26 genome in the presence and absence of plasmids pJAM4253 (rnj+) and pJAM202c (empty vector). Second, the tryptophan-inducible promoter PtnaA was used to control the expression of rnj in the H26 genome. To construct the plasmid for insertion of the T₇-PtnaA fragment upstream of rnj in the H26 genome, PCR amplified PtnaA from H. volcanii genomic DNA was fused to the 3′ end of the T7 terminator by overlap extension PCR. The resulting fragment was cloned into the XbaI and NdeI sites of pJAM1406. The 500 bp flanking fragment upstream of rnj was inserted into the AleI and XbaI sites of this intermediate plasmid to generate the final plasmid pJAM4259, which was propagated in GM2163 and then used to modify the genome of H26 by pyrE2-based homologous recombination to obtain strain HJ05. For complementation assay, HJ05 was separately transformed with plasmids pJAM1406 (+rnj-strepII) and pJAM202c (the empty vector). The strains were inoculated with single colony into 3 ml Hv-Min-Ura+ medium and grown to OD₆₀₀ of 0.6-0.8 in rotary shaker at 200 rpm and 42° C. till the OD₆₀₀ reached 0.6-0.8. Cells were subcultured from the starting OD₆₀₀ of 0.02 to OD₆₀₀ 0.6-0.8 with the same condition described above. This subculture step was repeated. The cells were then diluted to 10-2 to 10-7 in 18% saline water and spotted (20 μl each dilution) onto Hv-YPC plates. After spotting, the plates were air dried and incubated in the dark at 42° C. for 5 days.

1.2. Construction of H. volcanii Expression Plasmids

For preparation of the His6-RecJ3 expression plasmid pJAM4251, the gene fragment encoding recJ3 was amplified by PCR from H. volcanii genomic DNA with the primer set RecJ3F_NdeI and RecJ3TAAr_XhoI and cloned into plasmid pJAM503. A similar approach was used to construct the aRNase J-StrepII expression plasmid pJAM1406 using primer set HVO_2724 NdeI and HVO_2724 KpnI and cloning into plasmid pJAM809. To construct the expression plasmid pJAM4252, carrying tandem expression cassette p2.rrn: his6-recj3 following with p.fdx: rnj-strepII, an overlap extension PCR method was used to splice the promoter Pfdx and rnj-strepII (108); the resulting expression cassette was then cloned into the BlpI site of pJAM4251. Here a strong promoter, H. volcanii ferredoxin promoter Pfdx, was used for controlling expression of the rnj (aRNase J) gene. The sequencing confirmed plasmids were transformed into GM2163 to propagate dam⁻ genotype plasmids and transformed into H. volcanii for protein expression. His6-RecJ3 and aRNase J-StrepII were expressed from plasmid pJAM4252 in the host strain HJ02, which was constructed by deleting recJ3 and replacing the His-rich H. volcanii pitA with Natronomonas pharonis pitA (pitA_{Nph}) to avoid His rich PitA contamination during Ni²⁺ column chromatography as previously observed (109, 110). HJ07 was also used as a host strain for expression of His-RccJ3 and aRNase J-StrepII from pJAM4252 and is a Δrnj derivative of HJ02. The pitA_{Nph} replacement was also used to construct HJ04 (H26 ΔrecJ4 pitA_{Nph}).

### 1.3. Phleomycin and UV Stress Assays

Strains H164 (parent), H1999 (Δcdc48a), H1999-pJAM1410 (+cdc48a) and H1999-pJAM202c (empty vector) were used to examine cdc48a function. These strains were grown in 5 ml Hv-YPC medium to late log phase (OD₆₀₀ of ~0.8) in 13×100 mm culture tubes at 42° C. in an orbital shaker (200 rpm). Cells were subcultured to a starting OD₆₀₀ of 0.02 and grown to log phase (OD₆₀₀ of 0.4) in 5 ml Hv-YPC medium. Phleomycin (20 mg/ml stock in water) was added to final concentrations of 0, 0.5, 1.0 and 1.5 mg/ml in the 5 ml cultures for 1 h with intermittent orbital shaking. Deionized water was included as a mock control for comparison. The phleomycin treated cells were diluted to 10-2 to 10-7 in 30% saline water and spotted (20 μl per dilution) onto Hv-YPC plates. After spotting, the plates were air-dried and incubated in the dark at 42° C. (6 days). For UV treatment, the cells grown to log-phase in Hv-YPC medium were diluted in 30% saline water to 10-1 to 10-5 and spotted (20 μl per dilution) onto Hv-YPC plates. After spotting, the plates were air-dried and exposed to UV dosages (0, 50, 75, 80, 85 100 J/M2). Immediately after UV treatment, the plates were incubated in the dark at 42° C. (3 days). Strains H26 (parent), HS01 (ΔrecJ3), HS01-pJAM4251 (+his6-recJ3), HS01-pJAM4254 (+recJ3), and HS01-pJAM202c (empty vector) were used to examine recJ3 function. These strains were inoculated from isolated colonies into 3 ml Hv-Min-Ura+medium. Medium was supplemented with 0.3 μg/mL novobiocin for strains with plasmids. Cells were grown to late log phase (OD₆₀₀ of 0.6-0.8). Cells were subcultured to a starting OD₆₀₀ of 0.02 and grown to log phase (OD₆₀₀ of 0.5-0.6) in 3 ml Hv-Min-Ura+medium. The cells were treated in triplicate with phleomycin (2 mg/ml) in the 3 ml cultures for 1 h with intermittent orbital shaking. A mock control of equal volume of deionized water was included for comparison. After treatment, the cells were diluted to 10-2 to 10-7 in 18% saline water and were spotted (20 μl each dilution) onto Hv-YPC plates. After spotting, the plates were air dried and incubated in the dark at 42° C. for 3 days. A similar approach was used for comparative assay H164 (parent), H3931 (ΔrecJ3::trpA+), and H3932 (ΔrecJ4::trpA+) with the following modifications. Hv-YPC medium was used throughout the assay. After 1 h treatment with phleomycin (0, 0.5, 1 and 2 mg/ml), the cells were harvested and resuspended in fresh YPC medium prior to diluting/plating. All experiments described above were performed in triplicate and included biological triplicates within each experiment.

### 1.4. Protein Concentration and High Salt Strategy

Protein concentration was determined by Pierce BCA Protein Assay Kit (ThermoFisher Scientific, Waltham, MA, USA) using the supplied bovine serum albumin (BSA) as a standard. Protein purification methods that required complex stability and/or enzyme activity were performed in buffers supplemented with 2 M salt. The high salt was included in the buffer to maintain stability of the ‘salt-loving’ proteins common to the haloarchaea (63, 64, 111). Bovine pancreas DNase I (Alfa Aesar, Cat. No. J62229) was included in lysis buffers that was found active at in hydrolyzing H. volcanii genomic DNA (400 μg/mL) at conditions mimicking protein purification (i.e., DNase I at 4 μg/mL in high salt lysis buffer and incubations on ice for 6 h and room temperature for 2 h).

### 1.7. Ubl Purification

E. coli Rosetta (DE3)-pJAM1131 was used for purification of the Ubl SAMP1 with an N-terminal Flag-His6-tag (the Ubl of this study). The strain was freshly transformed and inoculated into LB medium supplemented with Km and Cm (500 ml medium per 2.8-L Fernbach flask). Cells were cultured at 25° C. (200 rpm). Isopropyl β-D-1-thiogalactopyranoside (IPTG) was added to a final concentration of 0.4 mM at log-phase (OD₆₀₀ 0.4-0.6 units) and cultivation was continued. After 8 h induction with IPTG, the cells were harvested by centrifugation (3000×g for 10 min, at 4° C.). The cells were washed in ice-chilled low salt buffer (20 mM HEPES, pH 7.5, 150 mM NaCl) and stored at −80° C. as cell pellets until lysis. Cells (3 g wet weight) were resuspended in 12 ml lysis buffer (20 mM HEPES, pH 7.5, 150 mM NaCl, 40 mM imidazole, 4 μg/mL DNase, 1 mM PMSF) and lysed thrice by French press (2,000 psi) (minimum high ratio of 140, Glen-Mills, NJ, U.S.). An equal volume of dilution buffer (20 mM HEPES, 4 M NaCl, pH 7.5) was added to the lysed cells. Cell lysate was clarified by centrifugation (9,200×g twice for 15 min, at 4° C.) and filtration (0.2 μm, cellulose acetate membrane). Protein sample was applied to a His Trap HP column pre-equilibrated in buffer (20 mM HEPES, pH 7.5, 2 M NaCl, 40 mM imidazole) and washed with the same buffer. The Ubl was eluted using 500 mM imidazole in 20 mM HEPES, 2M NaCl, and pH 7.5. The eluate was concentrated to a final volume of 500 μl using a Vivaspin centrifugal concentrator (3 MWCO, GE Healthcare). Concentrated samples were applied to a Superdex 75 HR 10/30 column (FPLC, GE Healthcare) equilibrated with HEPES high salt buffer (20 mM HEPES, 2 M NaCl, pH 7.5) at a flow rate of 0.2 mL/min, and 0.5 ml fractions were collected. Fractions containing the Ubl were pooled and stored at 4° C.

### 1.8. Ubl Coupling to Amine Reactive Beads

The Ubl was coupled to aldehyde-activated agarose beads (AminoLink Plus Resin) by reductive amination at room temperature according to supplier (Thermo Scientific, Waltham, MA, USA) with the following modifications. Immediately prior to coupling, the Ubl was dialyzed twice against high salt PBS buffer (2 M NaCl, 0.1 M sodium phosphate, pH 7.2) at 4° C. Likewise, the AminoLink Plus Resin (2 ml) was equilibrated by adding 3 resin-bed volumes of high salt PBS buffer at room temperature. The Ubl sample (5.0 and 8.5 mg protein) was added to the column resin (experimental replicates 1 and 2, respectively). Coupling was initiated by addition of 40 μl of 5 M NaCNBH₃ in 1 M NaOH. The mixture was rocked overnight at room temperature. After coupling, the flow through was collected to estimate the coupling efficiency of the Ubl through protein estimation. The remaining active sites were blocked by initial wash with 4 ml quenching buffer (1 M Tris HCl, 0.05% NaN₃, pH 7.5) and subsequent addition of 2 ml quenching buffer and 40 μl of 5 M NaCNBH₃ in 1 M NaOH. The sample was gently rocked at room temperature for 30 min. The column was washed with 10 ml (5 resin-bed volumes) of wash solution (2M NaCl, 0.05% NaN₃) to remove the non-coupled protein and unreactive cyanoborohydride. The Ubl amino groups available for covalent linkage resided in the N-terminal region of the polypeptide including the N-terminal α-amino group and ¿-amino groups of the lysine residues (Flag-tag K3 and K8 and SAMP1 K4). BSA (BCA protein standard) was similarly coupled to the beads as a control.

### 1.9. Cell Lysate Preparation for Ubl Pull-Down Assay

H. volcanii NH02-pJAM957, a Asamp1-3 AubaA mutant ectopically expressing UbaA-StrepII, was grown in ATCC974 medium with 100 mM DMSO to stationary phase at 42° C. (2×1 L culture in 2.8 L Fernbach flask, 200 rpm; derived from biological triplicate inoculum). Cells were harvested by centrifugation (3,000×g, 10 min at 4° C.) and stored at −20° C. as pellets (3 g wet weight) until use. On the day of the pull-down assay, the H. volcanii cell pellets were resuspended in ice cold high salt PBS buffer (0.1 M sodium phosphate buffer, pH 7.5, 2 M NaCl, 1 mM DTT, 1 mM PMSF, 4 μg/mL DNase) by pipetting in and out. Cells were lysed by French press (twice at 2,000 psi), and the cell lysate was clarified by centrifugation (16,264×g, 20 min at 4° C.). The supernatant was transferred carefully into a fresh tube without disturbing the pelleted cell debris. The cell-free extract was concentrated by dialysis against polyethylene glycol (PEG) 8000, where the cell free extract was packed in SnakeSkin Dialysis Tubing (3K MWCO, Thermo Scientific) and incubated at 4° C. in a container filled with PEG 8000. The cell free extract was concentrated to final volume of 3 ml, after which it was dialyzed against high salt PBS buffer (2 M NaCl, 0.1 M sodium phosphate, 0.2 mM DTT, pH 7.2) at 4° C. Immediately prior to pull-down assay, 5 mM ATP was added to the cell-free extract. Preparations that excluded the ATP supplementation were also analyzed for comparison.

### 1.10. Ubl Pull-Down Assay

The Ubl coupled beads were equilibrated with 3-column volumes (6 ml) of high salt PBS buffer. The cell lysate prepared for pull-down assay at 4° C. or on ice was applied to the Ubl-coupled column (3 mL total) at room temperature. The protein-bead slurry was incubated at 4° C. with gentle rocking (rocker M71015, Barnstead International, Dubuque, IA, USA) for 4 h per each 2 mL application. Washes were performed at 4° C. or on ice. The column was washed with 40 mL high salt PBS buffer (the flow through was collected to determine the binding efficiency). Proteins bound to the column were eluted at room temperature by addition of 8 mL of 0.1 M glycine-HCl buffer at pH 2.5 and collection as 1 mL fractions in tubes with 50-μL neutralization buffer (1M Tris-HCl, pH 8.5). Eluates were stored at −20° C. or −80° C. (long-term). Proteins were separated by SDS-PAGE and visualized by staining with SYPRO Ruby. Cell lysate was similarly applied to the BSA coupled beads to assess non-specific protein interactions. Gel slices of the protein bands that bound the Ubl beads were compared with the BSA control for protein identification by LC-MS/MS analysis.

### 1.11. Purification of RecJ3/4-aRNase J and RecJ3/4

For purification of the RecJ3/4-aRNase J complex, the following strains were used: H. volcanii HJ02-pJAM4251 expressing His-RecJ3 and H. volcanii HJ02- and HJ07-pJAM4252 strains expressing His-RecJ3 and aRNase J-StrepII. HJ02-pJAM202c served as the empty vector control (HJ07-pJAM202c is not viable, as rnj is essential). The strains were inoculated into 4 mL ATCC974 medium supplemented with novobiocin (0.3 mg/mL) until OD₆₀₀ 0.8-1.0. Cells were transferred to fresh medium (2-8×500-750 mL per 2.8-L Fernbach flask) supplemented with novobiocin and DMSO (100 mM). Cells were cultured at 42° C. (200 rpm) to stationary phase with OD₆₀₀ of 1.5-2.0. The cells were harvested by centrifugation at 3,000×g for 20 min at 4° C. and stored as pellets at −80° C. until used. Cells (3 g wet weight) were resuspended in 15 mL buffer (50 mM HEPES, pH 7.5, 2 M NaCl, 10% glycerol, 1 mM DTT, 4 μg/mL DNase, 5 mM MnCl₂, EDTA-free protease inhibitor mini tablets, 40 mM imidazole) and lysed thrice by French press at 2,000 psi (onto ice). Cell lysate was clarified by centrifugation at 13,177×g for 40 min at 4° C. and filtration (0.2 μm, cellulose acetate membrane). Cell lysate was applied to HisTrap HP column pre-equilibrated with binding buffer (50 mM HEPES, pH 7.5, 2 M NaCl, 10% v/v glycerol, 1 mM DTT, 5 mM MnCl₂, 40 mM imidazole) and washed with the same buffer. His-RecJ3 and its associates were eluted using elution buffer (binding buffer plus 460 mM imidazole) into 1 ml fractions.

For strains that also expressed RNaseJ-StrepII, the purified protein was dialyzed against dialysis buffer (50 mM HEPES pH 7.5, 2 M NaCl, 1 mM DTT, 5 mM MnCl₂, 5 mM MgCl₂) and then applied to Strep-Tactin resin pre-equilibrated with binding buffer (50 mM HEPES or 100 mM Tris-Cl, pH 7.5, 2 M NaCl, 1 mM DTT, 5 mM MnCl₂) by batch purification. The buffers were freshly prepared to avoid MnCl₂ precipitation. After washing 4 times with the binding buffer, the RecJ3/4-aRNase J complex was eluted with 1 mL elution buffer (binding buffer plus 5 mM desthiobiotin). His-trap fractions that did not bind the Strep-Tactin resin (flowthrough) were collected as RecJ3/4 subcomplexes. Protein fractions were dialyzed against dialysis buffer supplemented with 10% glycerol and concentrated using Vivaspin 500 centrifugal concentrators (Sartorius, Goettingen, Germany) as needed. RecJ3/4 subcomplexes were further purified by size exclusion chromatography in 50 mM HEPES, pH 7.5, 2 M NaCl, 1 mM DTT, 5 mM MnCl₂, 5 mM MgCl₂, and 10% glycerol. Purified proteins were analyzed by reducing 10% SDS-PAGE and nuclease activity assays. Buffers substituted with Tris-Cl and TCEP [tris(2-carboxyethyl) phosphine hydrochloride] for the HEPES and DTT, respectively, were found to yield similar results.

1.12. Purification of aRNase J Homodimer

For purification of the aRNase J homodimer, H. volcanii HS01-pJAM1406 that expresses aRNase J-StrepII was used. Cultures were initiated by adding 10 mL ATCC974 medium supplemented with novobiocin (0.3 mg/mL) to 2 plates of isolated colonies and transferring the resuspended cells to a 500 mL Erlenmeyer flask. Culture volume was adjusted to 100 mL final volume with the same medium, and cells were grown to OD₆₀₀ 0.8-1.0. This culture was used as a source of the 10 mL per flask inoculum into fresh ATCC medium (8×500 mL per 2.8-L Fernbach flask) supplemented with novobiocin and DMSO (100 mM). Cells were cultured at 42° C. (200 rpm) to stationary phase with OD₆₀₀ of 1.5-2.0. The cells were harvested by centrifugation at 3,000×g for 20 min at room temperature and stored as pellets at −80° C. until used. Cells (3 g wet weight) were resuspended in 15 mL buffer (50 mM HEPES, pH 7.5, 2 M NaCl, 1 mM TCEP, 4 μg/mL DNase, 5 mM MnCl₂, EDTA-free protease inhibitor at 1 tablet/50 mL) and lysed thrice by French press at 2,000 psi (onto ice). Buffers were freshly prepared to avoid MnCl₂ precipitation. Cell lysate was clarified by centrifugation at 13,177×g for 40 min at 4° C. and filtration (0.2 μm, cellulose acetate membrane). Cell lysate was applied to StrepTactin resin pre-equilibrated with binding buffer (50 mM HEPES, pH 7.5, 2 M NaCl, 1 mM TCEP, 5 mM MnCl₂) and washed with the same buffer. aRNase J-StrepII was eluted using elution buffer (binding buffer plus 5 mM desthiobiotin). Protein fractions were dialyzed against dialysis buffer (50 mM HEPES, pH 7.5, 2 M NaCl, 1 mM DTT, 5 mM MnCl₂), concentrated with Vivaspin 500 centrifugal concentrators, and further purified by size exclusion chromatography in the dialysis buffer. Purified protein was analyzed by reducing 10% SDS-PAGE and nuclease activity assay.

### 1.13. RecJ4 Role in RecJ3/4-aRNase J Complex Assembly

To evaluate the function of RecJ4 in assembly of the RecJ3/4-aRNase J complex, the tandem expression plasmid pJAM4252 was transformed into HJ04 to simultaneously produce His-RecJ3 and aRNase J in the absence of RecJ4. Purifications were performed successively with HisTrap and Strep-Tactin resin as described above. The host strain HJ04 was constructed by deleting recJ4 and replacing the His rich H. volcanii pitA with N. pharonis pitA.

### 1.14. Size Exclusion Chromatography (SEC) and Calibration

Samples were freshly dialyzed in SEC buffer (50 mM HEPES, pH 7.5, 2 M NaCl, 1 mM DTT, 5 mM MnCl₂, 5 mM MgCl₂, 10% glycerol for RecJ3/4, with MgCl₂ excluded for the aRNase J homodimer). Samples (0.5 mL) were applied at 0.3 mL/min to the Superdex 200 Increase 10/300 GL (SEC) column equilibrated in the same buffer. Fractions (0.5 mL) were collected and analyzed by 10% reducing SDS-PAGE. The SEC column was calibrated in low salt buffer (50 mM HEPES, pH 7.5, 150 mM NaCl, 1 mM DTT, 5 mM MnCl₂, 5 mM MgCl₂, 10% glycerol) using the following standards: blue dextran (void volume), ferritin (880 and 440 kDa), aldolase (158 kDa), alcohol dehydrogenase (150 kDa), bovine albumin (66 kDa), and thyroglobulin (669 and 1338 kDa) (Sigma).

### 1.15. Pull-Down Assays

i. aRNase J Pull-Down Assays.

H. volcanii H26 ‘wild type’ (H26, parent) and HS01 (ΔrecJ3 mutant) strains carrying plasmid pJAM1406 (rnj-strepII) and pJAM202c (empty vector control) were used for pull-down assays. Plasmid pJAM1406 was designed to express aRNase J with a C-terminal StrepII tag (-StrepII). Freshly isolated colonies were inoculated into ATCC974 medium supplemented with Nv and 100 mM DMSO (5 ml cultures) for 24 h and then transferred to fresh medium (0.5- to 1 L per 2.8 L Fernbach flask). Cells were cultured at 42° C. (200 rpm). At stationary phase (OD₆₀₀ of 2.5), cells (1 L culture) were harvested by centrifugation (3000×g for 15 min, at 4° C.) and washed in ice-chilled buffer (50 mM Tris-HCl, pH 7.5, 2M NaCl). Cells were stored as pellets at −80° C. until used. Cells (3 g wet weight) were resuspended in 12 mL lysis buffer (50 mM Tris-HCl, pH 7.5, 2M NaCl, 1 mM DTT, 4 μg/mL DNase, 0.5 mM MnCl₂, 4.5 mM MgCl₂ and EDTA-free protease inhibitor mini tablets, Pierce, Thermo Scientific, Waltham, MA, USA) and lysed thrice by French press (2,000 psi). Cell lysate was clarified by centrifugation (9,200×g twice for 15 min, at 4° C.) and filtration (0.2 μm, cellulose acetate membrane). For the H26 strains, the cell lysate was dialyzed twice for 2 h each time against 4 L buffer (50 mM Tris-HCl, pH 7.5, 2 M NaCl, 1 mM DTT, 0.5 mM MnCl₂, 4.5 mM MgCl₂) at 4° C. The dialyzed cell lysate of the H26 strains was transferred to ice and supplemented with 0.1 mM γATP (adenosine 5′-[γ-thio]triphosphate tetralithium salt, Sigma). For H26 and HS01 strains, the protein samples were applied to Strep-Tactin Superflow Plus (2 mL, Qiagen) pre-equilibrated in buffer (50 mM Tris-HCl, pH 7.5, 2 M NaCl, 1 mM DTT, 0.5 mM MnCl₂ and 4.5 mM MgCl₂) and washed with the same buffer. For H26 strains, proteins were eluted using an equal volume of 2×SDS-reducing buffer and boiling the Strep-Tactin beads (200 μL resin per liter of culture) for 6 min. Samples were centrifuged at 14,549×g (10 min at 21° C.). Proteins, in the supernatant, were separated by reducing 10% SDS-PAGE and analyzed by LC-MS/MS. For the HS01 strains, proteins were eluted from the Strep-Tactin beads using desthiobiotin. Eluted samples were dialyzed and concentrated using Vivaspin 500 centrifugal concentrators (Sartorius, Goettingen, Germany).

ii. RecJ4 Pull-Down Assay.

The RecJ4 pull-down assay was performed similarly to that of aRNase J isolated from H26 with the following exceptions. H. volcanii H26-pJAM1405 was used for purification of RecJ4-StrepII. Cell lysate was dialyzed twice for 2 h each time against 4 L buffer (50 mM Tris-HCl, pH 7.6, 2 M NaCl, 1 mM DTT, 10 mM MgCl₂) supplemented with 0.1 mM ATP (first dialysis) and 0.1 mM YATP (second dialysis). Protein sample was applied to a Strep-Tactin Superflow Plus resin (2 mL slurry, Qiagen) pre-equilibrated in buffer (50 mM Tris-HCl, pH 7.6, 2 M NaCl, 1 mM DTT and 10 mM MgCl₂) and washed with the same buffer prior to elution.

iii. Cdc48a Pull-Down Assay The Cdc48a pull-down assays were as above with the following exceptions. H. volcanii H1209-pJAM1409 was used for purification of His6-Cdc48a. Cells were washed in ice-chilled buffer (10 mM sodium phosphate buffer, pH 7.6, 2 M NaCl) prior to storing as pellets at −80° C. The lysis buffer was composed of 10 mM sodium phosphate buffer, pH 7.6, 2 M NaCl, 40 mM imidazole, 4 μg/mL DNase, protease inhibitor tablet, 1 mM DTT, 1 mM ATP and 5 mM MgCl₂. Protein samples were applied to a His Trap HP column (5 ml, GE Healthcare) pre-equilibrated in the lysis buffer (minus the protease inhibitor and DNase) and washed with this same buffer. The His6-Cdc48a protein was eluted by supplementing the equilibration buffer with 500 mM imidazole. The column eluates were boiled (6 min) in an equal volume of 2×SDS reducing buffer and centrifuged (14,549×g for 10 min at 21° C.).
1.16. Purification of H. volcanii RecJ3, RecJ4 and aRNase J Proteins from Recombinant E. coli

H. volcanii recJ3, recJ4 and rnj genes were subcloned into the plasmid vector pET28b. The recJ3 and recJ4 were fused to N-terminal His-tags while the rnj had a C-terminal StrepII tag. Expression plasmids (verified by DNA sequencing) were transformed into E. coli Rosetta (DE3) for synthesis of recombinant proteins. Isopropyl-β-D-1-thiogalactopyranoside (IPTG, 0.5 mM final concentration) was added to flask cultures (OD₆₀₀ of 0.6) to induce recombinant protein expression at 18° C. for 16 h. Cells were lysed, extract was clarified, and proteins were purified similarly to the H. volcanii purified proteins with the following modifications. Polyethylene glycol (PEG) 8000 was added to the clarified lysate at a final concentration of 5%, and the protein mixture was gently stirred for 15 min at 21° C. The pellet was discarded after centrifugation at 10,800×g for 15 min at 4° C. The concentration of PEG 8000 in the supernatant was increased to 15% and the protein fraction was precipitated. After centrifugation at 10,800×g for 15 min, the protein pellet was resuspended in the purification buffer to a final protein concentration of 10 mg/mL. For His-Trap chromatography, the column bound proteins were further washed with buffer supplemented 60 mM, 80 mM, and 100 mM imidazole before using elution buffer (binding buffer plus 460 mM imidazole) into 1 mL fractions. Purity of proteins was determined by 10% reducing SDS-PAGE.

### 1.17. Nuclease Activity Assays.

Purified proteins were examined for nuclease activity using 6-carboxyfluorescein (6-FAM) 5′- and 3′-end labeled oligonucleotide substrates under reaction conditions as detailed in figure legends. The 6-FAM labeled oligonucleotides were 23 bases of the sequence TTCGGCGACTGATGTTGATTGGC for DNA and UUCGGCGACUGAUGUUGAUUGGC for RNA (Table S1, Jia et al.). The substrates were synthesized and desalted by Integrated DNA Technologies (Coralville, Iowa, USA) with the 3′R substrate requiring RP-HPLC purification prior to assay. Protein concentration for assay was determined by bicinchoninic-acid assay (Pierce BCA Protein Assay) with bovine serum albumin as the standard according to supplier (ThermoFisher Scientific). To quench the nuclease activity assays, samples were rapidly transferred to ice and mixed with an equal volume of 2× loading buffer (90% (v/v) formamide, 100 mM EDTA, 0.2% (w/v) SDS, 10% (v/v) glycerol, 0.1% (w/v) bromophenol blue, and 0.1% (w/v) xylene cyanol). The mixture was heated for 5 min at 92° C., ice chilled for 5 min and separated by denaturing electrophoresis in 0.5×TBE (Tris-borate-EDTA) buffer (Invitrogen, Thermo Fisher Scientific, Waltham, MA, USA). Gels consisted of 20 or 30% (29:1) acrylamide/bisacrylamide, 8 M urea and 0.5×TBE buffer and were freshly prepared in an 8×10 cm mini vertical format using the Mini-PROTEAN Electrophoresis System (Bio-Rad, Hercules, CA, USA). Gels were subjected to pre-electrophoresis at 25 W for 30 min prior to sample application. Sample separation was at 15 W for 30 min. For further optimization, gels were pre-equilibrated at 30 W for 30 min (for 2 gels) and samples were separated by electrophoresis at 20 W for 26 min. 6-FAM fluorescence was detected in gel using the iBright FL1000 Imaging System (Thermo Fisher Scientific) at 488 nm excitation with smart exposure program. For enzyme kinetics (performed at least in triplicate), the resulting images were analyzed using ImageJ 1.53s software according to provider (112). Vₘₐₓ and k_{cat} calculations were performed by plotting data in Microsoft Excel. All results were found experimentally reproducible.

### 1.18. LC-MS/MS Analysis.

For analysis from gel slices, protein samples were separated by 10% SDS-PAGE, visualized by staining with Bio-Safe Coomassic (Bio-Rad, Hercules, CA, USA), and destained in double deionized water. SDS-PAGE gels were under reducing conditions with exception of the Ubl pull-down samples, which were analyzed under non-reducing conditions to examine non-covalent interactions. Unique protein bands were excised and analyzed by LC-MS/MS. Equivalent regions of the gel were similarly analyzed for the empty vector control. Proteins in the gel slices were treated with 45 mM dithiothreitol (DTT) and 100 mM 2-chloroacteamide (CAA). To minimize CAA carryover prior to trypsin digest, liquid was removed from the treated gel pieces and samples were washed with 25 mM ammonium bicarbonate buffer (pH >7.9), dehydrated by treatment with acetonitrile, and treated by centrifugal evaporation (SpeedVac, Labconco, Kansas City, MO, USA) to dryness. Samples were treated with trypsin (1 μg trypsin per 50 μg protein) at 37° C. for 15 h. Tryptic peptides were injected onto a capillary trap (Thermo Scientific PepMap) and desalted for 5 min with 0.1% (v/v) formic acid at a flow rate of 3 μL/min prior to loading onto a Thermo Scientific C18 Pep Map nanoflow high performance liquid chromatography (HPLC) column. The elution gradient of the HPLC column started at 3% solvent A (0.1% (v/v) formic acid, 3% (v/v) acetonitrile, and 96.9% (v/v) H₂O), 97% solvent B (0.1% (v/v) formic acid, 96.9% (v/v) acetonitrile, and 3% (v/v) H₂O) and finished at 60% solvent A, 40% solvent B using a flow rate of 300 nL/min for 30 min. LC-MS/MS analysis of the eluting fractions was carried out on an LTQ Orbitrap XL mass spectrometer (Thermo Scientific, San Jose, CA, USA). The instrument under Xcalibur 2.07 with LTQ Orbitrap Tune Plus 2.55 software was operated in the data dependent mode to automatically switch between MS and MS/MS acquisition. Full MS scans were acquired with a resolution of 60,000 in the Orbitrap from m/z 300-2000. Ten most intense ions were fragmented by collision-induced dissociation (CID) at a target value of 5,000 or maximum ion time of 150 ms. Dynamic exclusion was set to 60 seconds. Typical mass spectrometric conditions include a spray voltage of 2.2 kV, no sheath and auxiliary gas flow, a heated capillary temperature of 200° C., a capillary voltage of 44 V, a tube lens voltage of 165 V, an ion isolation width of 1.0 m/z, a normalized CID collision energy of 35% for MS2 in linear ion trap. The ion selection threshold was 500 counts for MS2. An activation q=0.25 and activation time of 30 ms were set. Raw data were analyzed using Mascot (Matrix Science, London, UK; version 2.2.2) against H. volcanii (Uniref UP000008243) and target decoy databases with the latter, including a set of reversed sequences generated by Mascot. Mascot was searched with a fragment ion mass tolerance of 0.8 Da and a parent ion tolerance of 15 ppm.

Carbamidomethylation of Cys was indicated as a fixed modification, while deamidation of Asn and Gln, oxidation of Met, and isopeptide linkage to Gly-Gly-were specified as variable modifications. Scaffold (Proteome Software Inc., Portland, OR, USA) was used to validate MS/MS based peptide and protein identifications, where protein probabilities were assigned by the Protein Prophet algorithm and peptide probabilities were assigned by the Peptide Prophet algorithm (113, 114). Protein identities and diglycine footprints were based on a threshold of 99.9% probability and <0.1% False Discovery Rate (FDR) and are summarized in Dataset S1, Jia et al. Normalized total spectral counts were calculated using the quantitative package associated with Scaffold (115).

### 1.19. RecJ3/4-aRNAse J Subunit Stoichiometry

The subunit stoichiometry of the RecJ3/4-aRNase J complex purified by TAP from HJ02-pJAM4252 was analyzed by absolute quantification (AQUA) based mass spectrometry. Peptides specific to tryptic fragments of aRNase J (382-IYDEIHVSGHLR-393), RecJ3 (137-QTGGPTVFR-145) and RecJ4 (286-AYPEVEVGDYVR-297) were synthesized with stable isotopes, where R represents arginine (¹³C₆, ¹⁵N₄) for each peptide. The labeled peptides were used as internal standards, and the absolute levels of the proteins were measured using selected reaction monitoring (SRM) in tandem mass spectrometry (MS/MS) analysis (Dataset S1D, Jia et al.) (116). Density scan was also used to estimate subunit stoichiometry. Proteins separated by reducing 10% SDS-PAGE stained in gel using SYPRO-Ruby or Coomassie Blue R-250 and visualized using an iBright FL1000 Imaging System. Protein band density was used to estimate protein quantity using ImageJ 1.53s software (112). The subunit stoichiometries determined by AQUA MS and ImageJ analysis were found comparable.

### 1.20. AQUA-Based MS Analysis

Protein concentration was determined by Bradford assay with bovine serum albumin as the standard. Protein (30 μg) was reduced with 40 mM DTT, alkylated with 100 mM of 2-chloroacetamide (CAA), and digested with trypsin/Lys-C protease mix (Promega Corporation, Madison, WI, USA; at an enzyme to protein ratio (wt/wt) of 1:100). The tryptic digests were desalted using micro ZipTip C-18 mini-reverse phase according to the manufacture manual (MilliporeSigma, Burlington, MA, USA). In brief, after wetting (with 50% acetonitrile) and equilibrating a ZipTip pipet tip (with 0.1% formic acid), the peptides were bound to C-18 material. A subsequent washing step with 0.1% formic acid was followed, and final elution was carried out with 80% acetonitrile and 0.1% formic acid. The samples were lyophilized to dryness at 160 mBar using a Labconco SpeedVac (Centrivap, Labconco Inc., USA). The resulting peptide pellets were stored at −20° C. until targeted MS analysis. A targeted method was developed for Selected Reaction Monitoring (SRM) to determine the absolute amount of protein in the sample on a TSQ Altis triple quadrupole mass spectrometer interfaced with a nano Easy 1200 ultra-performance liquid chromatography (UPLC) (Thermo Scientific, San Jose, CA, USA). Three unique peptides with heavy isotope were synthesized (Thermo Scientific, Pierce Biotechnology, Rockford, IL, USA) and each peptide optimized three transitions. The flow rate was set at 350 nL/min with solvent A (0.1% formic acid in water) and solvent B (0.1% formic acid and 99.9% acetonitrile) as the mobile phases. Separation was conducted using the following gradient: 2%-50% of B over 0-23 min; 50%-98% of B over 23-25 min, holding 98% of B over 25-27 min, and then from 90-0% of B from 27-28 min. The equilibration at 2% B was from 28 to 30 min. SRM conditions for peptides including m/z of SRM pairs (precursor and product pairs), collision energy, and RF lens voltage, were optimized by direct infusion of the compounds into the MS instrument at a concentration of 500 μg/mL, and the resulting optimized conditions are shown in Dataset S1D (Jia et al.). The Q1 and Q3 resolutions were 0.7 mass units FWHM (full width at half maximum). The dwell time of each SRM transition was 15.3 ms. The positive voltage on spray, ion transfer tube temperature, and CID gas (mTorr) were set at 2.4 kV, 325° C., and 1.5, respectively. Calibration standards were prepared by spiking the appropriate quantity of three heavy peptides to final concentrations of 40, 80, 120, 160, 200, 240, 280, 320, 400, 1000, 5000, and 8000 μg/mL. Heavy peptides were spiked to a final concentration of 400 μg/mL. Data processing was carried out using the Quan Brower module of the Xcalibur software ver. 4.1 (Thermo Scientific, San Jose, CA, USA) based on the calibration standards. Data processing was carried out using the Quan Brower module of the Xcalibur program (Thermo Scientific, San Jose, CA, USA), and extracted-ion chromatograms (XICs) of the peak area from standards were extracted and generated the calibration standards. The linear regression analysis was used for both constructions of the calibration curve and sample quantification.

### 1.21. SDS-PAGE and Immunoblotting Analysis

Protein samples were mixed with an equal volume of 2×SDS-PAGE loading buffer (100 mM Tris-HCl buffer at pH 6.8 with 4% (wt/v) SDS, 20% (v/v) glycerol, 0.6 mg/mL bromophenol blue, and 5% (v/v) β-mercaptoethanol). β-mercaptoethanol was excluded for non-reducing SDS-PAGE. Samples were boiled for 5-10 min and centrifuged at 12,000×g prior to separation by SDS-PAGE. Separated proteins were electroblotted from the gels onto PVDF membranes (0.45 μm) (MilliporeSigma, Burlington, MA, USA) as per standard protocol (BioRad, Hercules, CA, USA). HRP conjugated anti-His mouse monoclonal antibody (Proteintech Group Inc, Rosemont, IL, USA) and HRP-conjugated anti-StrepII monoclonal antibody (IBA Lifesciences, Germany) were used for immunoblotting analysis of His-tag and StrepII-tag respectively. To detect Cdc48a, polyclonal anti-VCP primary antibodies (AbCam product no. ab138298) and alkaline phosphatase (AP)-linked goat anti-rabbit IgG (H+L) secondary antibodies (SouthernBiotech, Birmingham, AL, USA) were used. AP activity was detected using CDP-Star (Applied Biosystems, Framingham, MA, USA). HRP activity was detected using ECL Plus immunoblotting substrate (Pierce, ThermoFischer Scientific, Waltham, MA, USA). Chemiluminescent signals were documented using X-ray film (Amersham Hyperfilm; Cytiva, Marlborough, MA, USA) or an iBright FL1000 (Thermo Fisher Scientific, Waltham, MA, USA).

### 1.22. Analysis of Genomic Co-Occurrence

InterPro categories were combined to define the RecJ3/4 homologs as requiring classification to the DHH phosphoesterase superfamily (IPR038763), the nucleic acid-binding OB-fold superfamily (IPR012340), and RNA-binding S1 domain (IPR022967). aRNase J homologs were of the aRNase J family (IPR004613) and were restricted to archaea. Similar restrictions were used to define RNA degradosomes, RNA exosomes and other related homologs as summarized in Table S2 and Data Set S2. Protein lists were downloaded from UniProtKB/TrEMBL using the advanced search engine (https://www.uniprot.org/) (117). To generate genome neighborhood networks (GNNs), protein lists were first entered as accession IDs into the Enzyme Similarity Tool (EFI-EST) web portal (https://efi.igb.illinois.edu/efi-est/) (118). The resulting dataset was assessed to determine the alignment score that would generate a sequence similarity network (SSN) at 40-50% amino acid sequence identity. This score was used to finalize and enter the SSN into the Genome Network Tool (EFI-GNT) for query by Pfam numbers to assess the co-occurrence of homologs within target protein (RecJ3, RecJ4 and aRNase J) defined genome neighborhoods. For Cdc48a and uncharacterized proteins of interest, the H. volcanii protein sequences (HVO_2380, HVO_2382 and HVO_1964) were entered into the sequence blast option of the EFI-EST web portal for generating the SSN used for GNN analysis. The criteria for inclusion in the GNN was that the homologs were detected within a window of 10 genes to occur in at least 70 archaeal genomes (with exception of the RecJ3-Cdc48 synteny noted in 26 methanogen genomes).

### 1.23. Data Availability.

Proteomic datasets are available through the PRoteomics IDEntifications (PRIDE) database (119) under accession numbers: PXD019896, PXD019897, PXD019898, PXD019906, PXD019931, PXD019932 and PXD019933.

### Example 2. Cdc48a, aRNase J and RecJ3/4 are Associated with Ubl Binding in H. volcanii

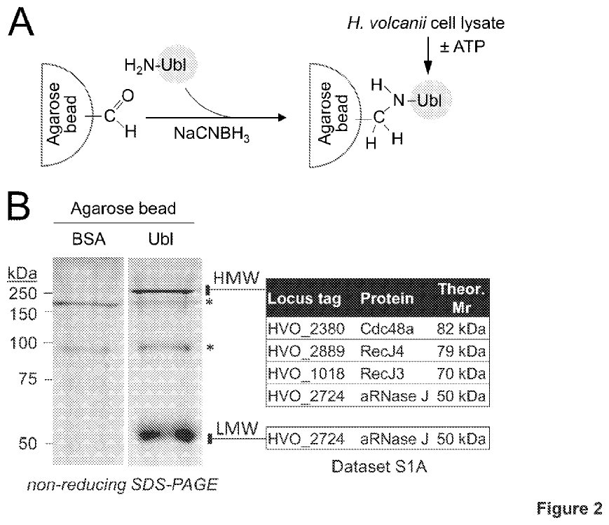

To identify Ubl interaction networks in archaea, the H. volcanii Ubl SAMP1 was covalently linked to agarose beads and used as bait to purify interacting proteins from H. volcanii cell lysate (FIG. 2A). Prior to lysis, the cells were grown to stationary phase in a medium supplemented with dimethylsulfoxide (DMSO), a condition known to stimulate Ubl-conjugate formation (54). Proteins that bound to the Ubl beads were separated by non-reducing SDS-PAGE, visualized by SYPRO Ruby staining, and identified by liquid chromatography tandem mass spectrometry (LC-MS/MS) analysis. Two major protein bands that bound the Ubl SAMP1 in the presence of ATP were identified: (i) a high-molecular-weight (HMW) band composed of Cdc48a, RecJ3, RecJ4, and aRNase J, and (ii) a low-molecular-weight (LMW) band of aRNase J (FIG. 2B; Data set S1A, Jia et al.). These proteins were not detected when the beads were coated with bovine serum albumin (BSA) or when ATP was omitted from the binding buffer. Genomic DNA did not appear to contribute to the binding, as DNase was included in the assay buffer. The ATP dependence of binding implicated Cdc48a, as Cdc48-type AAA ATPases are associated with Ub/Ubl pathways (55) and do so through ATP-dependent conformational shifts (56).

### Example 3. Phenotypic Analysis of Mutants Associated with Ubl Interactome

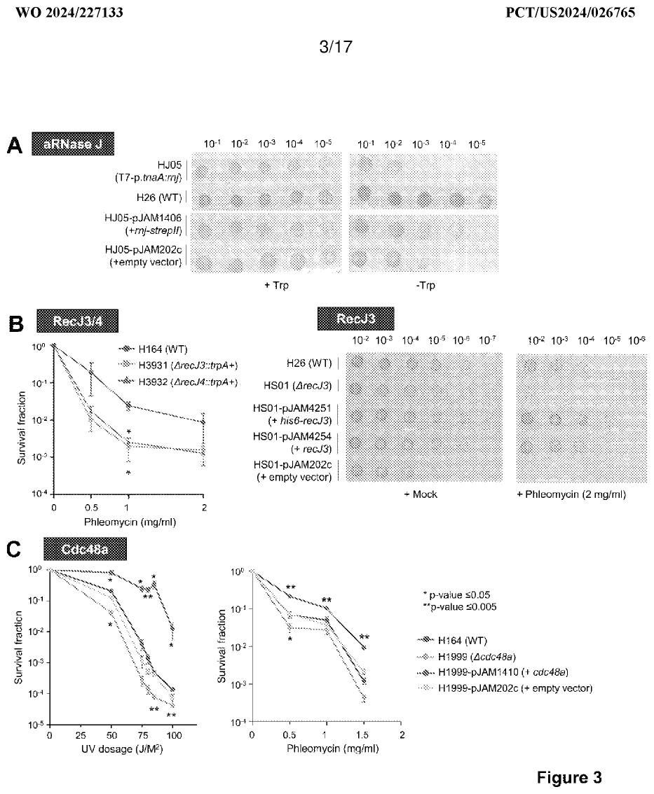

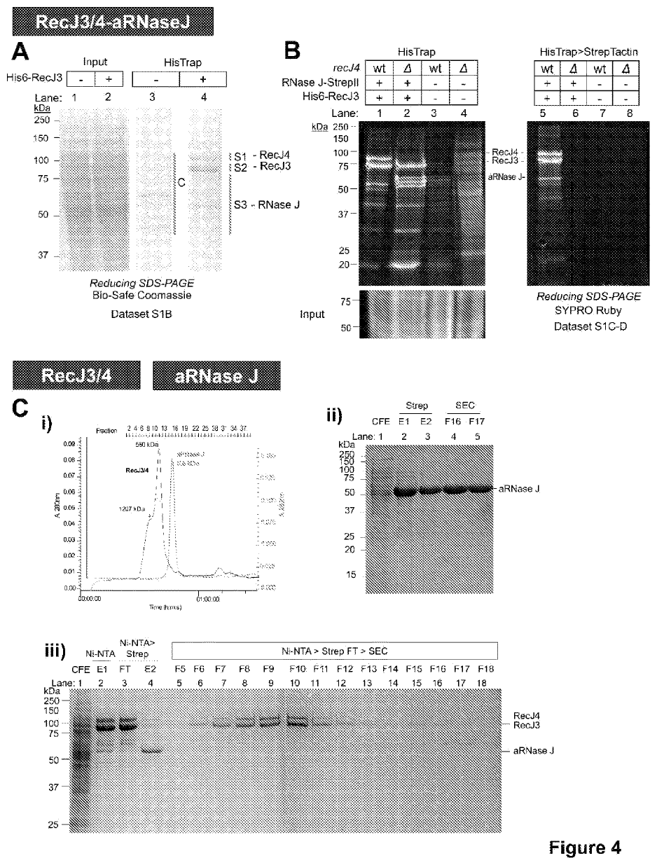

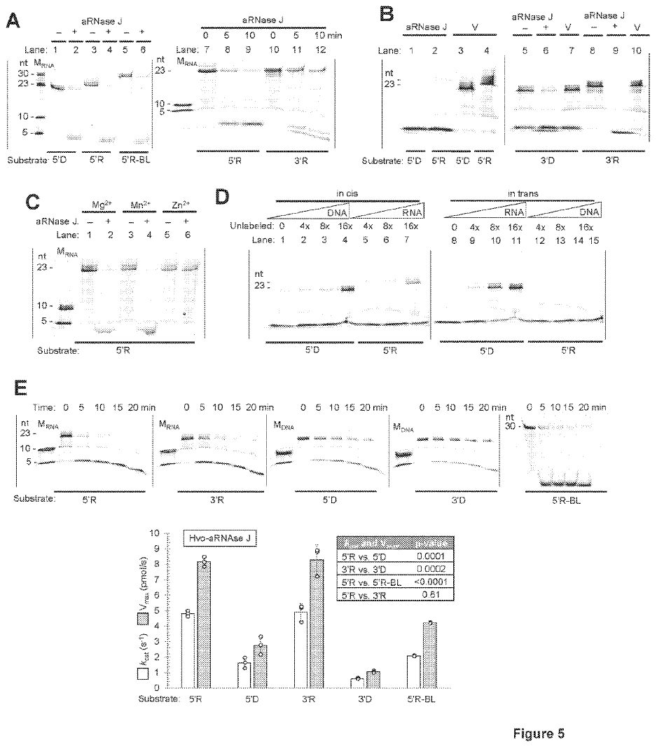

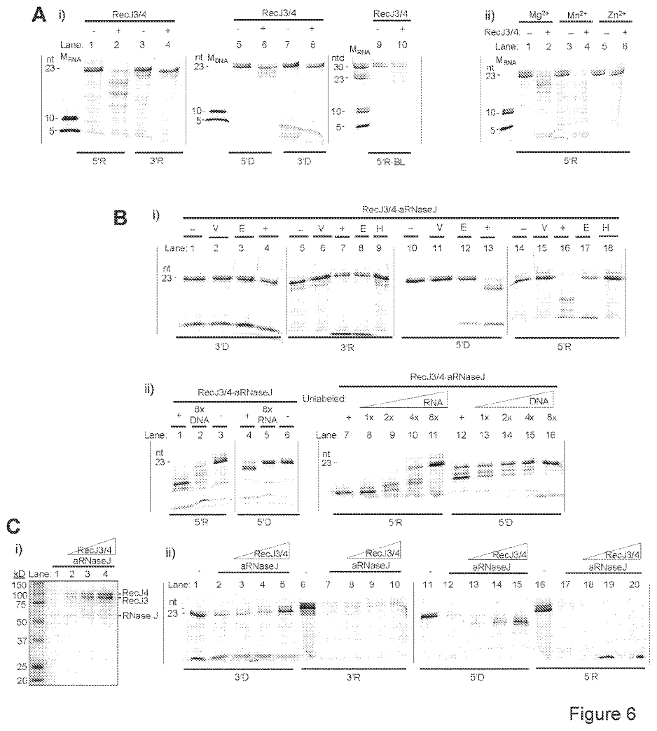

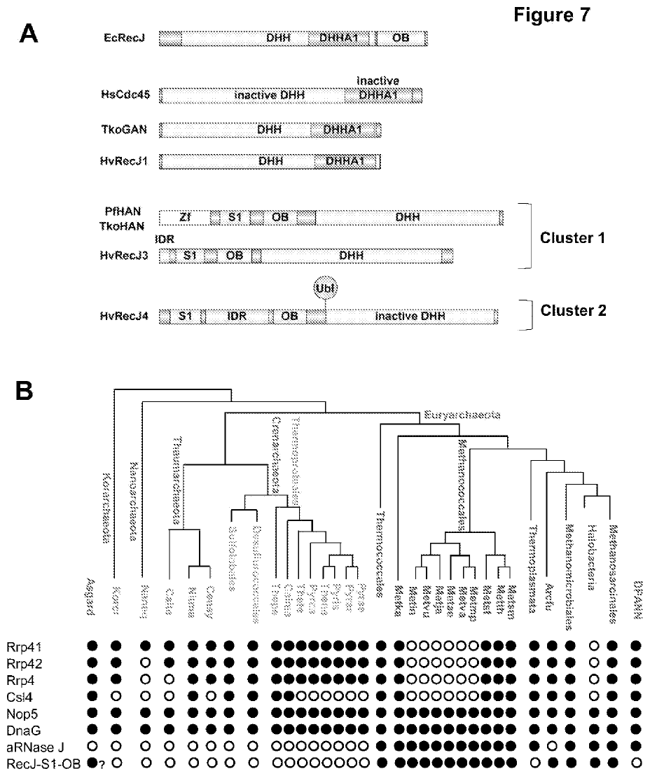

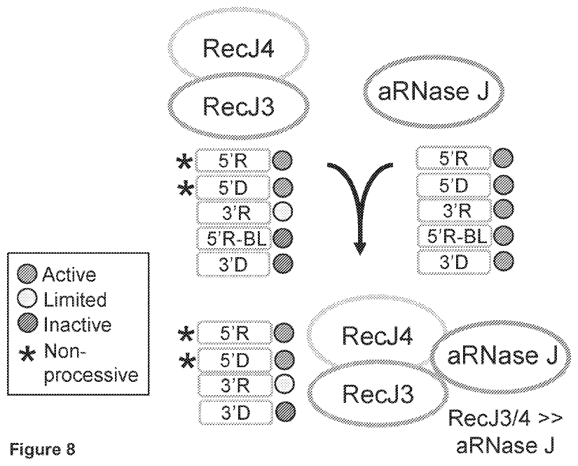

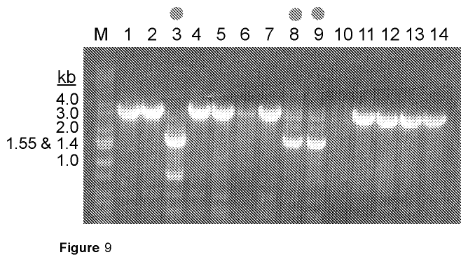

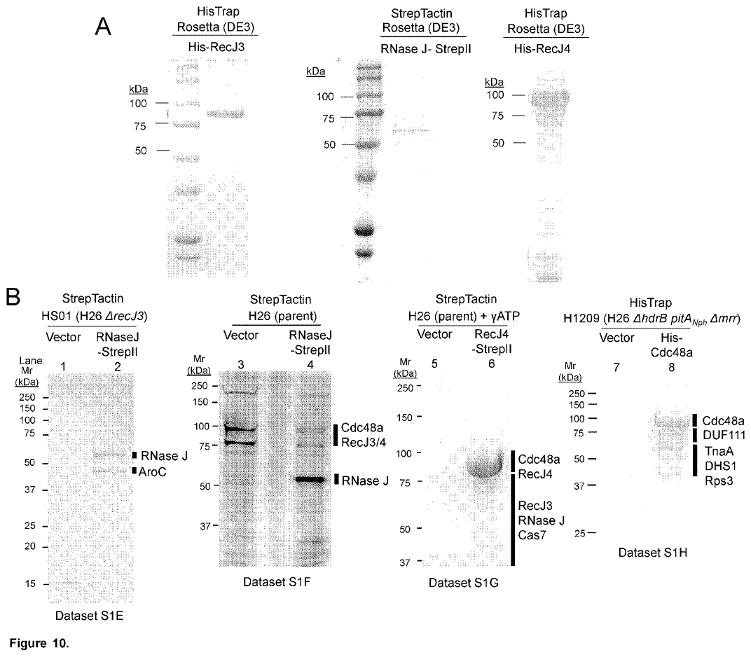

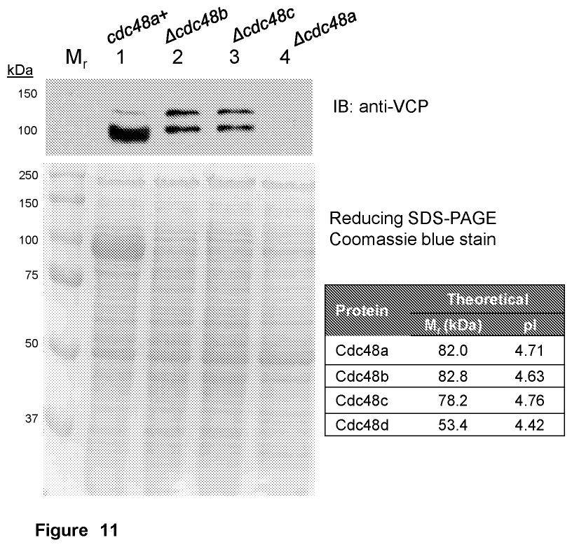

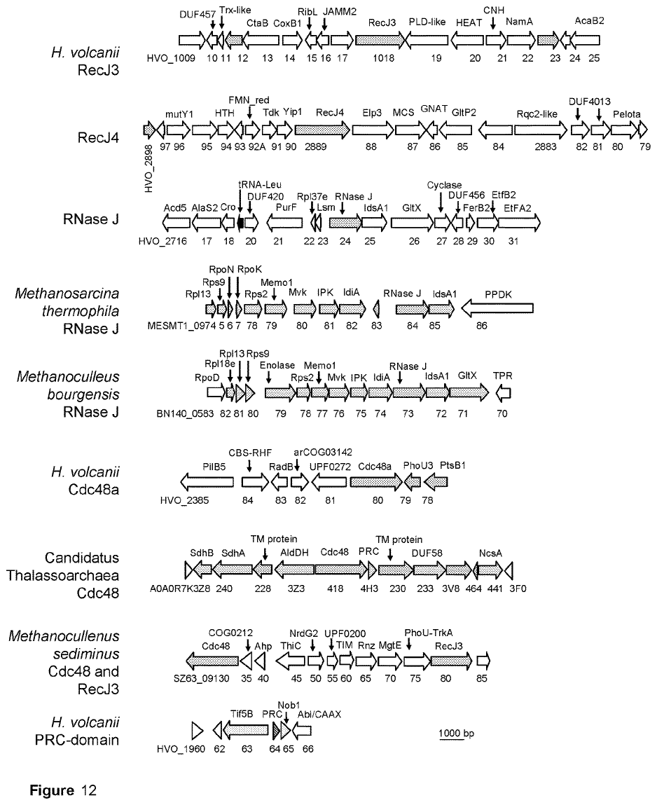

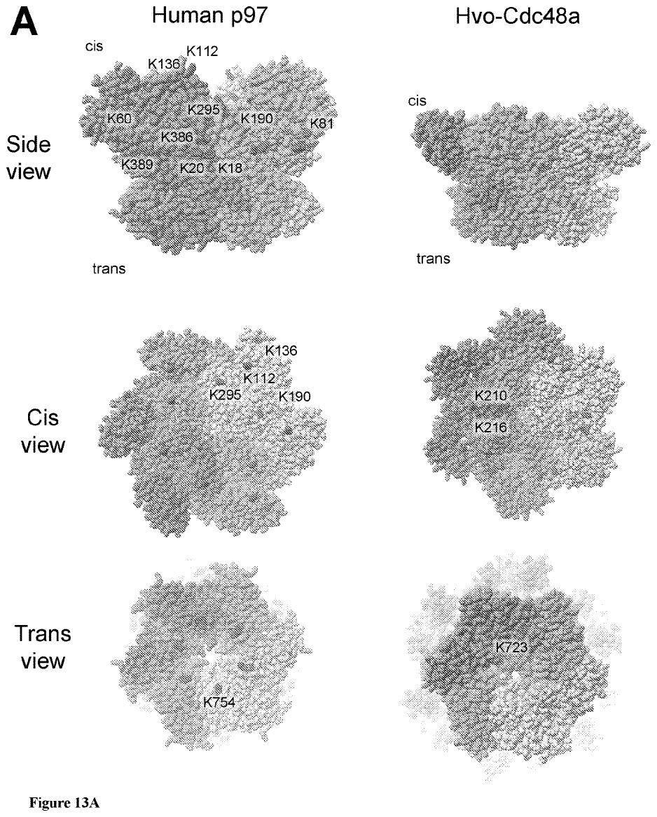

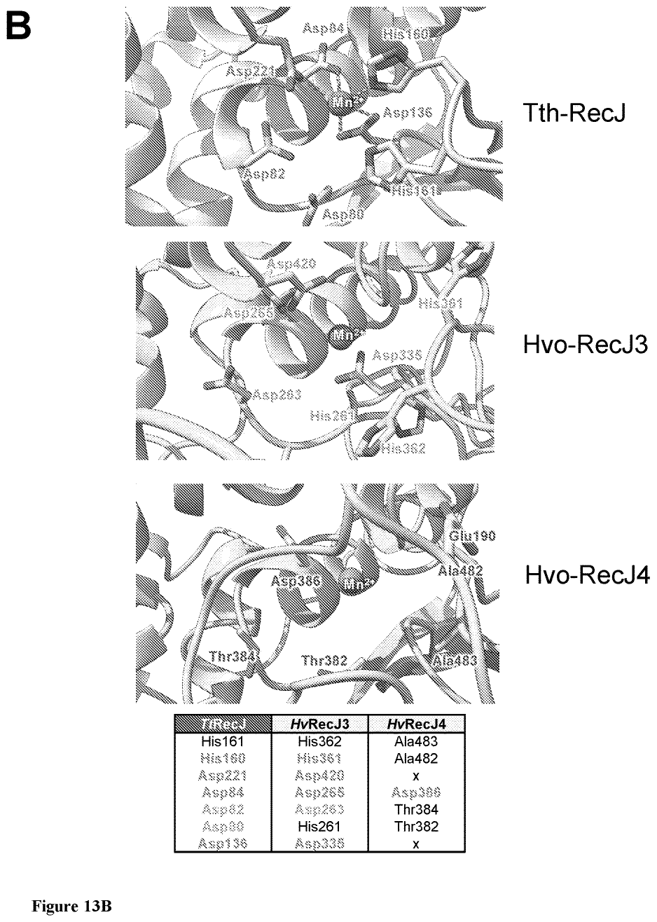

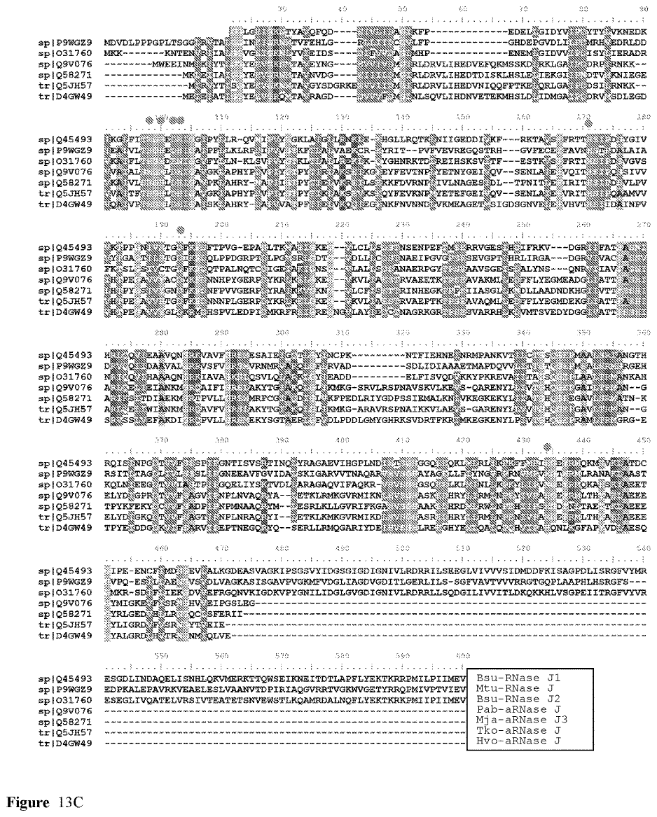

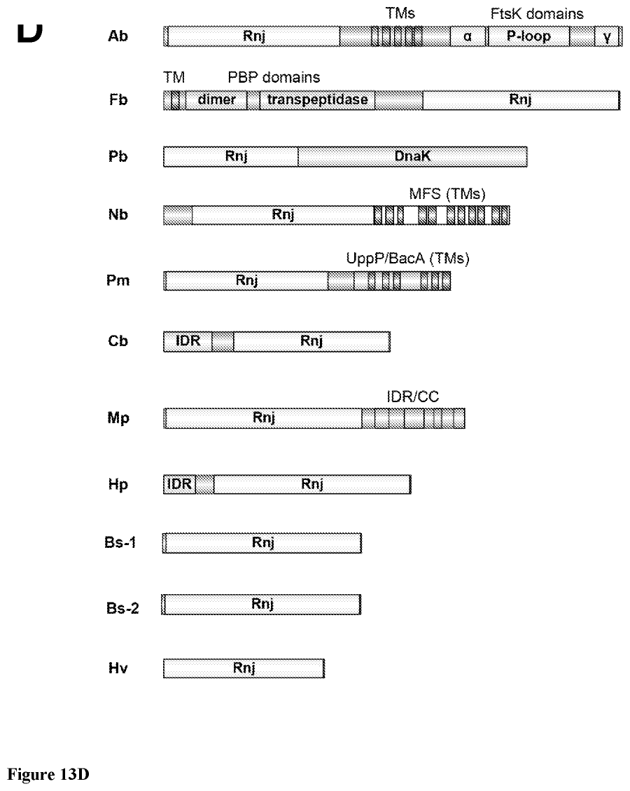

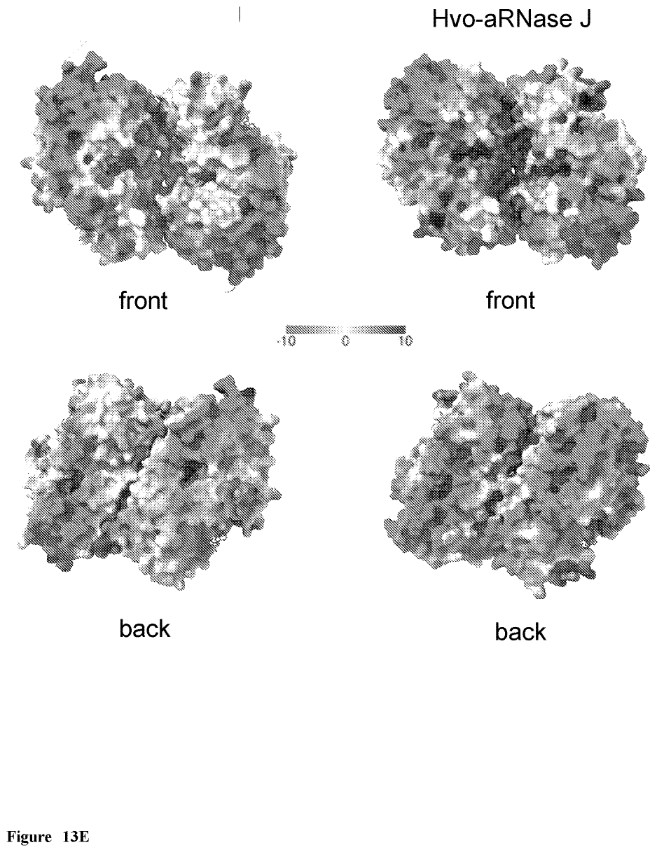

To assess the biological role of the Ubl interactome (RecJ3, RecJ4, aRNase J, and Cdc48a), the encoding genes were targeted for deletion, and the resulting mutants were analyzed for viability and recovery from DNA-damaging agents. aRNase J was suggested to be essential, as the encoding gene (rnj) could only be deleted from the genome when rnj was present on a plasmid. Additionally, the rnj+plasmid could not be cured from the Δrnj mutant (FIG. 9). To provide further evidence for the importance of rnj, a T7 terminator and tryptophan inducible promoter were integrated into the genome that allowed for the conditional expression of rnj in the presence (vs absence) of tryptophan. When compared to the parent, the resulting T7-p.tnaA:rnj strain was found to display wild-type growth in the presence of tryptophan and to have a pronounced reduction of growth in the absence of this amino acid (FIG. 3A). Growth of the p.tnaA:rnj strain under the tryptophan-depleted condition could be partially restored by ectopic expression of rnj-strepII (aRNase J-StrepII) but not by the empty vector control (FIG. 3A). Thus, through multiple genetic approaches, aRNase J was found to be essential in H. volcanii.

By contrast, the recJ3/4 genes were readily deleted (individually and together) from the H. volcanii genome and, thus, were not essential (Table S1, Jia et al.). Compared to the parent, the ΔrecJ3 and ΔrecJ4 mutants were reduced in survival after treatment with phleomycin, a DNA-damaging agent that introduces DSBs (FIG. 3B, left). The phleomycin concentration (0.5-2 mg/mL) was 1,000-fold greater than those used to examine bacterial mutants of DNA repair, such as E. coli (57), and may reflect the ability of haloarchaea to thrive in extreme environments that require robust DNA repair mechanisms (58). Ectopic expression of recJ3+ and his6-recJ3+ in the ΔrecJ3 mutant restored cellular recovery from phleomycin to levels compared to (if not more robust than) the parent (FIG. 3B, right).

Deletion of cdc48a was found to reduce the survival of cells exposed to phleomycin and ultraviolet (UV) radiation (FIG. 3C), with UV sensitivity similar if not more pronounced when compared to the H. volcanii DNA repair mutants Δcas1 and Δfen1 (59). Even more striking was the enhanced survival of cells from DNA-damaging agents when Cdc48a was expressed from a constitutive (p2.rrn) compared to native promoter (FIG. 3C).

Overall, aRNase J was found essential for growth, while Cdc48a, RecJ3, and RecJ4 were shown to be important for the recovery of cells from DNA-damaging agents.

### Example 4. RecJ3, RecJ4 and aRNase J form a Complex

To further investigate the Ubl interactome, an H. volcanii strain (HJ02-pJAM4251) was constructed that allowed for the purification of RecJ3 with an N-terminal His6-tag (His6-). Proteins uniquely bound to His6-RecJ3 were identified by comparison to an empty vector control (HJ02-pJAM202c). The predominant proteins, excised from reducing SDS-PAGE gels and analyzed by LC-MS/MS, were found to correspond to RecJ3, RecJ4, and aRNase J (FIG. 4A; Data set S1B, Jia et al).

To probe further into these protein-protein interactions, His6-RecJ3 and aRNase J-StrepII were expressed in H. volcanii (HJ02-pJAM4252) and isolated by tandem affinity purification (TAP) consisting of His-Trap and Strep-Tactin chromatography. The TAP fractions were directly analyzed by LC-MS/MS and found to be composed of three prominent proteins: RecJ3, RecJ4, and aRNase J (FIG. 4B, lane 5 vs 6; Data set SIC, Jia et al). AQUA-based MS/MS analysis estimated the RecJ3:RecJ4:aRNase J subunits to be in 2:2:1 stoichiometry (Data set SID, Jia et al.). “Low-abundant” proteins were also identified in these TAP fractions (vs empty vector) including homologs of DNA repair (UvrA and RadA), CRISPR-associated protein Cas7 (60), DnaK chaperone, and the β-CASP ribonuclease family homolog aCPSF1 (archaeal cleavage and polyadenylation specificity factor 1) (61). Overall, RecJ3, RecJ4, and aRNase J were found to be associated through protein-protein interactions by multiple strategies of purification.

### Example 5. RecJ4 as an Apparent Scaffold of the RecJ3/4-aRNase J Complex

RecJ4 appears non-catalytic based on the absence of conserved active site residues and may instead promote protein-protein interactions, as it has a large intrinsically disordered region (H104 to L236) predicted by AlphaFold (62). To examine whether RecJ4 is required for complex formation, His6-RecJ3 and aRNase J-StrepII were expressed in a ΔrecJ4 mutant and subjected to TAP. After purification by HisTrap, the RecJ4 band of ~90 kDa was notably absent from the His-RecJ3 fractions of the ΔrecJ4 mutant compared to the parent (FIG. 4B, lane 2 vs 1). Further purification by Strep-Tactin revealed aRNase J-StrepII to be absent from the ΔrecJ4 fractions compared to the parent (FIG. 4B, lane 6 vs 5). These results provide evidence that RecJ4 is important for the association of RecJ3 with aRNase J.

### Example 6. aRNase J and RecJ3/4 Subcomplexes

To further probe the subunits of the complex, multiple strategies were used to separately purify aRNase J, RecJ3, and RecJ4. Initially, these proteins were individually expressed and purified from recombinant E. coli (FIG. 10A) but were found inactive in hydrolyzing RNA and/or DNA even after dialysis into “high-salt” (e.g., 2 M NaCl) buffers. Haloarchaea use a “salt-in” strategy to maintain homeostasis in hypersaline conditions, and, thus, their proteins often require synthesis, purification, and storage in “high-salt” buffers for activity and stability (63-65). To overcome this hurdle, aRNase J was expressed with a C-terminal StrepII tag in an H. volcanii ΔrecJ3 mutant and purified to homogeneity by Strep-Tactin and size exclusion chromatography (SEC) (FIG. 4Ci and Cii). aRNase J purified in this manner was found to be associated as a 108-kDa homodimer.

To purify the RecJ3/4 subcomplex and ensure RNase J was not present in the preparation, the following strategy was used. The plasmid expressing His-RecJ3 and aRNase J-StrepII was transformed into the recJ3 mutant HJ02, and the rnj gene-encoding aRNase J (which is essential) was subsequently deleted from the genome. The resulting strain (HJ07) was used to ensure separate purification of His-RecJ3 from RNaseJ-StrepII by collecting proteins that bound the His-Trap column but flowed through the Strep-Tactin resin (FIG. 4Ciii, lane 3). These His-RecJ3 containing fractions were further purified by SEC and found to elute primarily as a 560-kDa subcomplex of RecJ3 and RecJ4 (FIG. 4Ci) in an equimolar ratio based on reducing SDS-PAGE analysis (FIG. 4Ciii, lanes 9-10). These results suggested RecJ3/4 form a tetramer of heterodimers (4×149 kDa), as the theoretical molecular masses of RecJ3 and RecJ4 are 70 and 79 kDa, respectively. Overall, these results reveal aRNase J to be a homodimer in the absence of RecJ3 and show RecJ3/4 to be associated in a large subcomplex of equimolar subunit ratio in the absence of aRNase J.

### Example 7. Nuclease Activity of RecJ3/4-RNase, aRNase J and RecJ3/4 Complexes

The aRNase J, RecJ3/4, and RecJ3/4-aRNase J complexes purified from H. volcanii were next examined for nuclease activity. Oligonucleotide substrates of ssDNA and RNA (23 nucleotides, nts) labeled at the 3′- and 5′-ends with 6-FAM (5′D, 3′D, 5′R, 3′R, see FIG. 5 legend) were used, in part, to distinguish 5′-3′ and 3′-5′ exonuclease activities, as this label should block exonuclease activity (66). The 3′6-FAM-labeled oligonucleotides have 5′ hydroxyl groups. Thus, the 3′R mimics RNA cleaved by metal-independent RNases (e.g., EC 4.6.1.18) and not nascent mRNA which commonly has a 5′ triphosphate group (67). An RNA substrate of 30 nts, labeled at the 5′-end with 6-FAM and including phosphorothioate modification in the body (BL, body label), was also included to examine endonuclease activity. This latter substrate (5′R-BL, see FIG. 5 legend) was designed to block 5′ exoribonuclease activity and to distinguish between enzymes that have only 3′-5′ exonuclease activity vs those that have endonuclease activity. A product of 21 nts should accumulate if the enzyme has 3′-5′ exonuclease activity and lacks endonuclease function. Alternatively, the formation of products <21 nts would indicate the enzyme has endonuclease activity.

In the homodimeric configuration, aRNase J was found to be an endonuclease that hydrolyzed all RNA and ssDNA substrates examined (FIG. 5). The hydrolyzed products were found to be less than 5 nts (FIG. 5A). The nuclease activities could be attributed to aRNase J, as proteins similarly purified from the empty vector control were not active (FIG. 5B). aRNase J was found active when Mg²⁺ or Mn²⁺ (vs Zn²⁺) was included in the assay (FIG. 5C). Hydrolysis of the 5′-end-labeled ssDNA by aRNase J was reduced when unlabeled ssDNA or RNA was added in excess to the reaction (FIG. 5D, lanes 2-4, 9-11). By comparison, aRNase J-mediated hydrolysis of the 5′end-labeled RNA (5′R) was only diminished by the addition of unlabeled RNA and not ssDNA (FIG. 5D, lanes 5-7, 12-14). These results suggested aRNase J preferred RNA over ssDNA. To further probe these findings, kinetic values were determined for aRNase J. Reaction velocities (k_{cat} and Vₘₐₓ) were found to be from highest to lowest according to substrate as 5′R and 3′R>5′R-BL>5′D>3′D (FIG. 5E), revealing RNA to be a preferred substrate over ssDNA. Overall, aRNase J, when purified independent of RecJ3/4, was found to be a homodimeric Mn²⁺/Mg²⁺-dependent nuclease that harbored endonuclease activity with preference for RNA over ssDNA as a substrate. Thus, aRNase J was not restricted to 5′-3′ exoribonuclease activity.

The RecJ3/4 subcomplex was next examined for nuclease activity. RecJ3/4 was found to be most active as a 3′-5′ exonuclease with an apparent preference for RNA over ssDNA (FIG. 6Ai, lane 2 vs 6). The primary products, detected for the RecJ3/4-mediated hydrolysis of RNA and ssDNA in the 3′-5′ direction, were >10 nt. The RecJ3/4 subcomplex was found to have limited if any 5′-3′ exonuclease activity against RNA or ssDNA (FIG. 6Ai, lanes 4 and 8) and to have no RNA endonuclease activity based on finding RNA products >21 nts accumulated when assayed with the 5′R-BL substrate (FIG. 6Ai, lane 10). Further analysis for metal dependence revealed the RecJ3/4 complex was functional in the presence of Mg²⁺ or Mn²⁺ but not Zn²⁺ (FIG. 6Aii). Thus, when compared to aRNase J, the RecJ3/4 subcomplex had a similar divalent metal requirement of Mg²⁺ or Mn²⁺ (vs Zn²⁺); however, RecJ3/4 had limited, if any, 5′-3′ exo- or endonuclease activity, and instead hydrolyzed RNA and ssDNA by a 3′-5′ exonuclease mechanism that appeared non-processive.

The nuclease activity of the RecJ3/4-aRNase J complex with 2:2:1 subunit stoichiometry was also analyzed. The complex was found to display activities that mirrored the RecJ3/4 subcomplex (FIG. 6Bi). RecJ3/4-aRNase J preferentially hydrolyzed 5′-end-labeled RNA and ssDNA oligonucleotides with limited, if any, hydrolysis observed for the 3′-end-labeled RNA or ssDNA substrates (FIG. 6Bi). These activities were specific to RecJ3/4-aRNase J when compared to protein fractions similarly purified from the empty vector control strain, and were sensitive to heat and EDTA treatments (FIG. 6Bi). Further examination revealed RecJ3/4-aRNase J hydrolysis of the 5′-end-labeled ssDNA substrate (5′D) was reduced when examined in the presence of excess unlabeled RNA or ssDNA (FIG. 6Bii, lanes 5 and 16). Likewise, RecJ3/4-aRNase J hydrolysis of the 5′-end-labeled RNA substrate (5′R) was substantially reduced in the presence of excess unlabeled RNA (FIG. 6Bii, lane 11); however, the hydrolysis of 5′R appeared simulated by excess unlabeled ssDNA (FIG. 6Bii, lane 2 vs 1). These results suggest RecJ3/4-aRNase J functions primarily as a 3′-5′ exonuclease, and prefers RNA over ssDNA as a substrate (with ssDNA potentially stimulating the RNase activity)

### Example 8. RecJ3/4 Impact on aRNase J Nuclease Activity

The nuclease activity profiles of the complex and subcomplexes suggested that the RecJ3/4 may influence aRNase J activities. To further investigate this possibility, increasing amounts of RecJ3/4 were added to the aRNase J homodimer and then assayed for nuclease activity (FIG. 6Ci). As the ratio of RecJ3/4 to aRNase J was increased, hydrolysis of the 3′-end-labeled ssDNA and RNA substrates (3′D and 3′R) by endonuclease and/or 5′-3′ exonuclease activities was found to be reduced (FIG. 6Cii). Hydrolysis of the 5′-end labeled ssDNA (5′D) also appeared to shift to the formation of larger nt products (FIG. 6Cii). By contrast, hydrolysis of the 5′-end-labeled RNA (5′R) by 3′-5′ exonuclease activity was found to be robust even at high ratios of RecJ3/4 to aRNase J (FIG. 6Cii). These results suggest that RecJ3/4 influences aRNase J activity and results in a complex that functions primarily as a 3′-5′ exoribonuclease, with the non-processive 3′-5′ hydrolysis of ssDNA also observed.

### Example 9. Cdc48a is Loosely Associated with the RecJ3/4-aRNase J Complex

The Ubl interactome was further probed by analyzing various affinity-purified protein fractions by MS/MS. In brief, aRNase J-StrepII was found to co-purify with RccJ3/4 and Cdc48a (FIG. 10B; Data set S1F, Jia et al.). RecJ4-StrepII was detected in association with RecJ3, aRNase J, Cdc48a, and Cas7 (FIG. 10B; Data set S1G, Jia et al.). His6-Cdc48a was identified to bind a wide variety of proteins including those of translation and PTM pathways (FIG. 10B; Data set S1H, Jia et al.) consistent with other AAA ATPases that notoriously interact with diverse protein partners and substrates (68, 69). Overall, these pull-down results provided additional evidence that RecJ3, RecJ4, and aRNase J form a complex that may transiently associate with Ubl tags through Cdc48a.

### Example 10. RecJ4 and Cdc48a are Ubl Modified

Diglycine remnants (+114 Da) were detected on the lysine residues of tryptic peptides derived from RecJ4-StrepII and His6-Cdc48a. The mass spectra were supported by the b- and y-ion series for each peptide (Dataset S1I, Jia et al.). These remnants mapped to Cdc48a K210, K216, and K723 and RecJ4 K358. The H. volcanii strains used for protein purification encoded Ubl SAMP2, which has a -KGG C-terminal tail that would generate these footprints. Thus, Cdc48a and RecJ4 appeared samp2ylated (conjugated to SAMP2). The sites were not fully occupied by the Ubl modifier suggesting regulation, and the lysine residues were found conserved among species (Dataset S1J-K, Jia et al.). To provide further evidence for these post-translational modifications, cell lysate was separated by reducing SDS-PAGE and analyzed by immunoblotting using antibodies raised against the related Cdc48 VCP. In H. volcanii ‘wild type’ cells, Cdc48a was detected as two distinct bands that were not detected in the Δcdc48a mutant (FIG. 11). The faster migrating band that may be the unmodified form of Cdc48a was found to be highly abundant in the cdc48a+ overexpression strain (FIG. 11) . These results are consistent with Ubl modification of Cdc48a. Interestingly, other proteins of the Ubl interactome are found Ubl modified in H. volcanii (53, 54, 61, 70).

### Example 11. Co-Occurrence Patterns Reveal RecJ3/4-aRNase J Homologs are Conserved in Archaea that Lack an RNA Exosome

To understand the phylogenetic relationships of the RecJ3/4-aRNase J complex, co-occurrence patterns were compared to the archaeal RNA exosome. Taxonomic distribution patterns of ASH-Ski2, aRNase J, and Rrp41/Cs14 (RNA exosome) homologs among archaea have been analyzed by the previous study (13). Here, criteria were newly formulated to group RecJ3/4 homologs based on distinctions in protein domain architecture (FIG. 7A; Data set S2A, Jia et al.). In this scheme, RecJ3/4 homologs were classified based on the presence of an RNA-binding S1 domain (IPR022967) and clustering to DHH phosphoesterase (IPR038763) and nucleic acid-binding OB-fold (IPR012340) superfamilies. RecJ3/4 homologs meeting these criteria were found only in certain archaea and were not present in eukaryotes, bacteria, or viruses. aRNase J classification was based on the established IPR004613 family, with phylogenetic distribution as previously described (13). By this approach, co-occurrences of RecJ3/4 and aRNase J homologs were found to be restricted to euryarchaeota and to complement archaea missing the canonical RNA exosome including all haloarchaea and certain methanogens (FIG. 7B)

### Example 12. Genome Synteny

As archaeal RNA exosomes are found commonly encoded in genomic neighborhoods with machinery for protein degradation, transcription, and translation (71), next it was examined whether the newly identified RecJ3/4-aRNase J complex had similar associations (FIG. 12; Table S2; Data Set S2, Jia et al.). RecJ3, RecJ4, aRNase J, and Cdc48a gene homologs were found in genomic neighborhoods with homologs of the Ubl-proteasome system, RNA degradation, DNA recombination/repair, translation, transcription, and other important cellular functions. Interestingly, RecJ3 and Cdc48 gene homologs were found in apparent operons in some methanogens, and gene homologs of Cdc48 and PRC-barrel domain proteins used in RNA processing (72) were also found in genomic synteny. Thus, while the Rec3/4-aRNase J complex differed in subunit composition from the RNA exosome, it still appeared encoded on archaeal genomes with pathways of protein degradation, transcription, translation, and other related functions.

Sequences
>X-protein-seq-RecJ3_HVO_1018
(SEQ ID NO: 1)
MSDEHAGDSGDDSGPNSDARPIVYDLAPNCTADDVETDAYYHAVVNGVVEYGIFVDV
SDSVSGLIHESNLSADYEVGDRLVVRLESVRDNGDIAFAEDTPDDYRTLTVDHEPTITPIS
SLSVGESVTVEGVVTQIKQTGGPTVFRIADDSGIVAAAAFEEAGVRAFPGVDLDDVVRM
AGTVEDHEGTRQLEVDGLTVLDDEAAADARQRIDAALDERAEPEPVEPLVEWSAFEKL
RDDLEDVARLLRRTVLEGRPIRVRHHADGDGMCASVPVQLALERLITEVHDDPDAPRH
LFKRLPSKAPFYEMEDVTRDLNFALEGRARHGQRLPFLLMLDNGSTEEDVPAYENLAH
YDVPIAVVDHHHPDPEAVDPLLDAHVNPYLHDEDYRITTGMMCVELARMIHPDVTDEL
RHVPAVAGLSDRSKAEVMDNYIALAEDEGYDREQLLDVGEALDYAAHWLRYNDGASI
VNDVLNVGCDDEERHRELVEFLSTRAERDVDRQLEAAEPHLEHERLDSGANLYRIDLD
KWAHRFTYPAPGKTTGKLHDRKVQETGEPVITIGYGPDFAVLRSDGVRLDIPRMVAELN
EEVVGGGVSGGGHLVVGSIKFVSGMREEVIDSLVEKMAEADIDEELSTTA
>Y-protein-seq-RecJ4-HVO_2889
(SEQ ID NO: 3)
MDWITHEEDVWFEFRGNSPHQLVPGRFYRGTVDGYADFGVFVDLASNVTGLLHRSELD
RRLESLDWEPGDEVFVQVKNVRDNGNIDLGWSIRQSDSEFRGARIHDPDGDADGQPVE
QDAESGGPTTVKTRPKTGKTTKPAGVSTSSEQTESDADAEPEPEPKPESDAGDEDRAPT
AGDVVDDIAANGESEQEADAEPESEPESDSESKEDAESDDEQEVETDADAESADEAERV
TLASIDDRVGDVIRVEGEIASVRQTGGPTVFELRDETAIADCAAFVEAGVRAYPEVEVGD
YVRIDGEVERRRGELQIETEELTILDGDEADTVAQRLADALSDEARPDAVAPLAAHEPV
AAVGKSLLDAAEAIRRAVLESRPIVVRHTATADGYVAGAAVERAVLPLIREEHPRDDAE
YHYFTRRPLEEAVYGMDAATNDVTRMLEDRDRHDEKLPLVLLLGAGSTAESLDGLGLL
GVYGSERVVVDAAPADDEVAAEVDVLVNPAREGADARDLSVGALASTLSVAVNDDVR
DDVSHLPAVSYWENCPQQYLDLAESHGFDVDRVRELREAVALEAYYQSYQDKRELIAD
LLFDADEGLAGHVSEQFRIKLEDEIETAQANLERREVGAISAAVLDSDAYSHRFDFPPTG
LLVDELHRRTREGDAFVTVALGMDELYLRATGDLDLRAVVESAAEKAPAAGLAAAGIR
EGRIEFLTGARDEALEAVLDAAAEQF
>Z-protein-seq-aRNaseJ_HVO_2724
(SEQ ID NO: 5)
MEIEIATIGGYEEVGRQMTAVRAGDDVVVFDMGLNLSQVLIHDNVETEKMHSLDLIDM
GAIPDDRVMSDLEGDVQAIVPTHGHLDHIGAISKLAHRYDAPVVATPFTIELVKQQIEGE
NKFNVNNDLVKMEAGETMSIGDSGNVELEFVHVTHSIIDAINPVVHTPEGAVVYGLDKR
MDHSPVLEDPIDMKRFREIGREGNGVLAYIEDCTNAGRKGRTPSESVARRHLKDVMTSV
EDYDGGIVATTFSSHISRVSSLVEFAKDIGRQPVLLGRSMEKYSGTAERLGFVDLPDDLG
MYGHRKSVDRTFKRIMKEGKENYLPIVTGHQGEPRAMLTRMGRGETPYEIDDGDKVIF
SARVIPEPTNEGQRYQSERLLRMQGARIYDEIHVSGHLREEGHYEMLQALQPQHVIPAH
QNLKGFAPYVDLAESQGYALGRDLHVTRNGNMIQLVE
>A-DNA-seq-RecJ3_HVO_1018
(SEQ ID NO: 2)
ATGAGCGACGAGCACGCCGGGGATTCCGGCGACGACTCGGGTCCGAATTCGGACGC
ACGACCTATCGTCTATGATCTCGCACCGAACTGTACCGCCGACGACGTCGAGACCGA
CGCGTACTACCACGCCGTCGTCAACGGCGTCGTGGAGTACGGCATCTTCGTCGACGT
CTCCGACTCCGTCTCCGGACTCATCCACGAGTCCAACCTCTCGGCCGACTACGAGGT
CGGGGACCGACTGGTCGTCCGCCTCGAATCCGTCCGCGACAACGGCGACATCGCGT
TCGCCGAGGACACGCCCGACGACTACCGCACCCTGACCGTCGACCACGAGCCGACC
ATCACGCCTATCTCCAGTCTCTCGGTCGGCGAGTCGGTCACGGTCGAAGGCGTCGTC
ACGCAGATTAAGCAGACCGGCGGCCCGACCGTCTTCCGCATCGCCGACGACTCCGG
CATCGTCGCCGCCGCCGCCTTCGAGGAGGCCGGCGTCCGCGCGTTCCCCGGTGTCGA
CCTCGACGACGTGGTCCGCATGGCCGGCACCGTCGAAGACCACGAGGGCACCCGAC
AGCTCGAAGTCGACGGCCTCACCGTCCTCGACGACGAGGCCGCCGCCGACGCCCGC
CAGCGCATCGACGCCGCGCTCGACGAGCGCGCCGAGCCGGAACCGGTCGAACCGCT
CGTGGAGTGGTCGGCGTTCGAGAAGCTCCGCGACGACCTCGAAGACGTGGCGCGCC
TGCTCCGCCGGACGGTCCTCGAAGGCCGCCCGATTCGCGTCCGCCACCACGCCGACG
GCGACGGCATGTGCGCGTCCGTCCCGGTCCAACTGGCCCTCGAACGGCTCATCACCG
AGGTCCACGACGACCCCGACGCGCCGCGACACCTGTTCAAGCGCCTGCCGAGCAAG
GCCCCGTTCTACGAGATGGAGGACGTGACCCGCGACCTGAACTTCGCGCTCGAAGG
TCGCGCCCGCCACGGCCAGCGGCTTCCCTTCCTGCTCATGCTCGACAACGGGTCGAC
CGAGGAGGACGTGCCCGCCTACGAGAACCTCGCGCACTACGACGTGCCCATCGCGG
TCGTCGACCACCACCACCCCGACCCCGAGGCGGTCGACCCCCTCCTCGACGCGCACG
TCAACCCGTACCTCCACGACGAGGACTACCGCATCACGACCGGCATGATGTGCGTC
GAACTCGCGCGGATGATTCACCCCGACGTGACCGACGAACTGCGTCACGTCCCCGC
CGTCGCCGGGCTGTCCGACCGCTCGAAGGCGGAGGTCATGGACAACTACATCGCGC
TCGCCGAAGACGAGGGCTACGACCGCGAACAGCTCCTCGACGTGGGCGAGGCGCTC
GACTACGCCGCCCACTGGCTGCGCTACAACGACGGCGCGTCTATCGTCAACGACGT
GCTCAACGTCGGCTGTGACGACGAGGAGCGCCACCGCGAACTCGTCGAGTTCCTCTC
GACGCGCGCCGAGCGCGACGTGGACCGACAGCTCGAAGCCGCGGAGCCGCACCTCG
AACACGAGCGCCTCGACAGCGGGGCCAACCTCTACCGCATCGACCTCGACAAGTGG
GCCCACCGCTTTACCTACCCCGCGCCGGGCAAGACCACGGGCAAGCTCCACGACCG
CAAGGTCCAGGAGACGGGCGAGCCGGTCATCACCATCGGCTACGGTCCCGACTTCG
CCGTCCTCCGCTCCGACGGCGTCCGCCTCGACATCCCGCGGATGGTCGCCGAACTGA
ACGAGGAAGTCGTCGGCGGCGGCGTCTCCGGCGGCGGCCACCTCGTCGTCGGCTCC
ATCAAGTTCGTCAGCGGCATGCGCGAGGAGGTCATCGACAGCCTCGTCGAGAAGAT
GGCCGAAGCCGACATCGACGAAGAGCTGTCGACGACGGCGTAA
>B-DNA-seq-RecJ4_HVO_2889
(SEQ ID NO: 4)
ATGGATTGGATTACGCACGAGGAGGACGTCTGGTTCGAATTCCGGGGCAATAGCCC
CCACCAGCTCGTCCCCGGCCGATTTTACAGAGGCACCGTCGACGGGTACGCCGACTT
CGGTGTCTTCGTCGACCTCGCTTCGAACGTGACTGGCTTACTGCACCGCAGCGAACT
CGACCGCCGCCTCGAATCCCTCGACTGGGAGCCGGGCGACGAGGTGTTCGTTCAGG
TGAAGAACGTCCGCGACAACGGCAACATCGACCTCGGCTGGTCGATTCGCCAGTCC
GACTCGGAGTTCCGCGGCGCTCGCATCCACGACCCCGACGGCGACGCCGACGGCCA
GCCCGTCGAGCAGGATGCCGAGAGCGGCGGTCCGACGACCGTCAAGACGCGCCCGA
AGACGGGCAAGACGACGAAGCCCGCCGGCGTGAGCACGTCGTCCGAGCAGACCGA
GTCCGACGCCGACGCGGAGCCCGAACCCGAACCCAAACCCGAGTCCGACGCCGGAG
ACGAGGACCGCGCGCCGACCGCCGGCGACGTCGTCGACGACATCGCCGCAAACGGC
GAGTCCGAGCAGGAAGCCGACGCGGAGCCGGAGAGTGAGCCCGAGAGCGACTCGG
AGTCCAAAGAGGACGCCGAGTCTGACGACGAGCAGGAAGTCGAGACCGACGCTGA
CGCCGAGTCCGCCGACGAGGCGGAGCGCGTGACGCTCGCCAGCATCGACGACCGCG
TCGGCGACGTGATTCGCGTCGAGGGCGAAATCGCCAGCGTCCGCCAGACCGGCGGC
CCGACGGTGTTCGAACTCCGCGACGAGACGGCCATCGCAGACTGCGCGGCGTTCGT
CGAGGCCGGCGTCCGCGCGTACCCCGAAGTCGAAGTGGGCGACTACGTCCGCATCG
ACGGCGAGGTCGAGCGCCGCCGCGGCGAACTCCAAATCGAGACCGAGGAGCTGAC
GATTCTCGACGGCGACGAGGCCGACACCGTGGCCCAGCGCCTCGCGGACGCCCTCT
CGGACGAGGCCCGCCCGGACGCCGTCGCGCCCCTCGCGGCCCACGAGCCGGTCGCC
GCCGTCGGCAAGTCGCTGCTCGACGCCGCGGAGGCCATCCGCCGCGCCGTGCTCGA
ATCGCGCCCCATCGTCGTCCGCCACACGGCCACCGCCGACGGCTACGTCGCCGGCGC
GGCCGTCGAGCGCGCCGTGCTCCCGCTCATCCGTGAGGAGCACCCCCGCGACGACG
CCGAGTACCACTACTTCACCCGTCGCCCGCTCGAAGAGGCTGTCTACGGCATGGACG
CCGCCACGAACGACGTGACGCGGATGCTCGAAGACCGCGACCGCCACGACGAGAA
GCTCCCGCTCGTGCTTCTCCTCGGTGCCGGTTCGACCGCCGAGTCCCTCGACGGTCTC
GGCCTGCTCGGCGTCTACGGCTCCGAGCGCGTCGTCGTCGACGCCGCGCCCGCCGAC
GACGAGGTCGCCGCGGAAGTCGACGTGCTCGTCAACCCCGCCCGCGAGGGGGCCGA
CGCCCGCGACCTCTCGGTTGGCGCGCTCGCCTCCACGCTTTCCGTCGCCGTCAACGA
CGACGTGCGCGACGACGTGTCACACCTCCCCGCGGTCAGCTACTGGGAGAACTGCC
CACAGCAGTACCTCGACCTCGCGGAATCGCACGGCTTCGACGTCGACCGCGTCCGC
GAACTCCGCGAGGCCGTCGCGTTGGAAGCCTACTACCAGTCGTATCAGGACAAGCG
CGAACTCATCGCAGACCTGCTGTTCGACGCCGACGAGGGCCTCGCCGGCCACGTCTC
CGAGCAGTTCCGCATCAAGCTCGAAGACGAAATCGAGACGGCGCAGGCGAACCTCG
AACGCCGCGAGGTCGGTGCCATCTCCGCGGCCGTCCTCGACTCCGACGCTTACAGTC
ACCGCTTCGACTTCCCGCCGACGGGCCTGCTCGTGGACGAACTCCACCGCCGCACCC
GTGAGGGCGACGCCTTCGTGACCGTCGCGCTCGGCATGGACGAACTCTACCTCCGCG
CCACGGGCGACCTCGACCTCCGCGCGGTCGTCGAGTCGGCCGCCGAGAAAGCGCCC
GCCGCCGGCCTCGCCGCCGCGGGCATCCGCGAAGGTCGCATCGAGTTCCTGACCGG
CGCGCGCGACGAGGCGCTCGAAGCCGTCCTCGACGCCGCCGCCGAGCAGTTCTAA
>C-DNA-seq-aRNaseJ_HVO_2724
(SEQ ID NO: 6)
ATGGAAATCGAAATCGCAACCATAGGCGGATACGAAGAAGTCGGCCGTCAGATGAC
GGCTGTCCGTGCCGGAGACGACGTCGTCGTCTTCGACATGGGTCTCAACCTGTCGCA
GGTCCTCATCCACGACAACGTCGAGACCGAAAAGATGCACAGCCTCGACCTCATCG
ACATGGGCGCTATCCCGGACGACCGGGTCATGAGCGACCTCGAAGGAGACGTGCAG
GCCATCGTCCCCACGCACGGCCACCTCGACCACATCGGTGCCATCTCCAAGCTCGCC
CACCGCTACGACGCCCCCGTCGTGGCGACGCCCTTCACCATCGAACTGGTGAAACA
GCAGATCGAAGGCGAGAACAAGTTCAACGTCAACAACGACCTCGTCAAGATGGAAG
CCGGCGAGACGATGTCCATCGGCGACTCCGGCAACGTGGAACTCGAATTCGTCCAC
GTCACCCACTCCATCATCGACGCCATCAACCCGGTCGTCCACACGCCCGAGGGCGCT
GTCGTCTACGGTCTCGACAAGCGCATGGACCACTCGCCGGTCCTCGAAGACCCCATC
GACATGAAGCGCTTCCGCGAAATCGGTCGCGAGGGCAACGGCGTGCTCGCGTACAT
CGAAGACTGTACGAACGCCGGCCGGAAGGGCCGCACGCCCTCCGAGTCCGTCGCGC
GCCGCCACCTCAAAGACGTGATGACCTCCGTCGAGGACTACGACGGCGGCATCGTC
GCGACGACGTTCTCGTCGCACATCTCCCGGGTCTCCTCGCTCGTCGAGTTCGCCAAG
GACATCGGCCGCCAGCCGGTCCTCCTCGGCCGCTCGATGGAGAAGTACTCCGGCAC
GGCCGAGCGCCTCGGCTTCGTCGACCTCCCCGACGACCTCGGGATGTACGGCCACCG
CAAGTCCGTCGACCGCACCTTCAAGCGAATCATGAAGGAGGGCAAGGAGAACTACC
TGCCCATCGTCACGGGCCACCAGGGCGAGCCGCGCGCGATGCTCACCCGCATGGGC
CGCGGCGAGACGCCGTACGAGATTGACGACGGCGACAAGGTCATCTTCTCGGCGCG
GGTCATCCCGGAGCCGACGAACGAGGGCCAGCGCTACCAGTCCGAACGCCTCCTGC
GCATGCAGGGCGCGCGCATCTACGACGAGATTCACGTCTCCGGCCACCTCCGAGAG
GAGGGCCACTACGAGATGCTCCAGGCGCTCCAGCCCCAGCACGTCATCCCGGCTCA
CCAGAACCTGAAAGGCTTCGCTCCGTACGTGGACCTCGCGGAGAGTCAGGGCTACG
CCCTCGGTCGCGACCTCCACGTCACGCGGAACGGCAACATGATTCAGCTGGTGGAG
TGA

### REFERENCES

1. Yang W. 2011. Nucleases: diversity of structure, function and mechanism. Q Rev Biophys 44:1-93. https://doi.org/10.1017/-S0033583510000181
2. Mohanty B K, Kushner S R. 2016. Regulation of mRNA decay in bacteria. Annu Rev Microbiol 70:25-44. https://doi.org/10.1146/annurev-micro-091014-104515
3. Tourrière H, Chebli K, Tazi J. 2002. mRNA degradation machines in eukaryotic cells. Biochimie 84:821-837. https://doi.org/10.1016/s0300-9084(02)01445-1
4. Gagliardi D, Dziembowski A. 2018. 5′ and 3′ modifications controlling RNA degradation: from safeguards to executioners. Philos Trans R Soc Lond B Biol Sci 373:20180160. https://doi.org/10.1098/rstb.2018.0160
5. Hawley B R, Lu W T, Wilczynska A, Bushell M. 2017. The emerging role of RNAs in DNA damage repair. Cell Death Differ 24:1989-1989. https://doi.org/10.1038/cdd.2017.146
6. Yates L A. 2021. Regulation of DNA break repair by RNA. Prog Biophys Mol Biol 163:23-33. https://doi.org/10.1016/j.pbiomolbio.2020.12.005
7. Dorléans A, Li de la Sierra-Gallay I. Piton J. Zig L, Gilet L. Putzer H, Condon C. 2011. Molecular basis for the recognition and cleavage of RNA by the bifunctional 5′-3′ exo/endoribonuclease RNase J. Structure 19:1252-1261. https://doi.org/10.1016/j.str.2011.06.018
8. Dutta T, Deutscher M P. 2009. Catalytic properties of RNase BN/RNase Z from Escherichia coli: RNase BN is both an exo- and endoribonuclease. J Biol Chem 284:15425-15431. https://doi.org/10.1074/jbc.M109.005462
9. Phung D K. Rinaldi D, Langendijk-Genevaux P S, Quentin Y, Carpousis A J, Clouet-d'Orval B. 2013. Archaeal β-CASP ribonucleases of the aCPSF1 family are orthologs of the eukaryal CPSF-73 factor. Nucleic Acids Res 41:1091-1103. https://doi.org/10.1093/nar/gks1237
10. Deutscher M P. 2015. How bacterial cells keep ribonucleases under control. FEMS Microbiol Rev 39:350-361. https://doi.org/10.1093/femsre/fuv012
11. Evguenieva-Hackenberg E. 2011. The archaeal exosome. Adv Exp Med Biol 702:29-38. https://doi.org/10.1007/978-1-4419-7841-7_3
12. Gauernack A S, Lassek C, Hou L, Dzieciolowski J, Evguenieva-Hackenberg E, Klug G. 2017. Nop5 interacts with the archaeal RNA exosome. FEBS Lett 591:4039-4048. https://doi.org/10.1002/1873-3468.12915
13. Phung D K, Etienne C, Batista M, Langendijk-Genevaux P, Moalic Y, Laurent S, Liuu S, Morales V, Jebbar M, Fichant G, Bouvier M, Flament D, Clouet-d'Orval B. 2020. RNA processing machineries in archaea: the 5′-3′ exoribonuclease aRNase J of the β-CASP family is engaged specifically with the helicase ASH-Ski2 and the 3′-5′ exoribonucleolytic RNA exosome machinery. Nucleic Acids Res 48:3832-3847. https://doi.org/10.1093/nar/gkaa052
14. Clouet-d'Orval B, Rinaldi D, Quentin Y, Carpousis A J. 2010. Euryarchaeal β-CASP proteins with homology to bacterial RNase J have 5′- to 3′-exoribonuclease activity. J 285:17574-17583. Biol Chem https://doi.org/10.1074/jbc.M109.095117
15. Zheng X, Feng N, Li D, Dong X, Li J. 2017. New molecular insights into an archaeal RNase J reveal a conserved processive exoribonucleolysis mechanism of the RNase J family. Mol Microbiol 106:351-366. https://doi.org/10.1111/mmi.13769
16. Levy S, Portnoy V, Admon J, Schuster G. 2011. Distinct activities of several RNase J proteins in methanogenic archaea. RNA Biol 8:1073-1083. https://doi.org/10.4161/rna.8.6.16604
17. Li J, Hou Y, Gu X, Yue L, Guo L, Li D, Dong X. 2020. A newly identified duplex RNA unwinding activity of archaeal RNase J depends on processive exoribonucleolysis coupled steric occlusion by its structural archaeal loops. RNA Biol 17:1480-1491. https://doi.org/10.1080/15476286.2020.1777379
18. Li de la Sierra-Gallay I, Zig L, Jamalli A, Putzer H. 2008. Structural insights into the dual activity of RNase J. Nat Struct Mol Biol 15:206-212. https://doi.org/10.1038/nsmb.1376
19. Even S, Pellegrini O, Zig L, Labas V, Vinh J, Bréchemmier-Baey D, Putzer H. 2005. Ribonucleases J1 and J2: two novel endoribonucleases in B. subtilis with functional homology to E. coli RNase E. Nucleic Acids Res 33:2141-2152. https://doi.org/10.1093/nar/gki505
20. Mora L, Ngo S, Laalami S. Putzer H. 2018. In vitro study of the major Bacillus subtilis ribonucleases Y and J. Methods Enzymol 612:343-359. https://doi.org/10.1016/bs.mie.2018.08.004
21. Raj R, Nadig S, Patel T, Gopal B. 2020. Structural and biochemical characteristics of two Staphylococcus epidermidis RNase paralogs RNase J1 and RNase J2. J Biol Chem 295:16863-16876. https://doi.org/10.1074/jbc.RA120.014876
22. Pei X Y, Bralley P. Jones G H, Luisi B F. 2015. Linkage of catalysis and 5′ end recognition in ribonuclease RNase J. Nucleic Acids Res 43:8066-8076. https://doi.org/10.1093/nar/gkv732
23. Newman J A, Hewitt L, Rodrigues C, Solovyova A, Harwood C R. Lewis R J. 2011. Unusual, dual endo- and exonuclease activity in the degradosome explained by crystal structure analysis of RNase J1. Structure 19:1241-1251. https://doi.org/10.1016/j.str.2011.06.017
24. Šiková M, Wiedermannová J, Převorovský M, Barvík I, Sudzinová P, Kofroňová O, Benada O, Šanderová H, Condon C, Krásný L. 2020. The torpedo effect in Bacillus subtilis: RNase J1 resolves stalled transcription complexes. EMBO J 39: e102500. https://doi.org/10.15252/embj.2019102500
25. Nagata M, Ishino S, Yamagami T, Simons J-R, Kanai T, Atomi H, Ishino Y. 2017. Possible function of the second RecJ-like protein in stalled replication fork repair by interacting with Hef. Sci Rep 7:16949. https://doi.org/10.1038/s41598-017-17306-0
26. Lovett S T, Kolodner R D. 1989. Identification and purification of a singlestranded-DNA-specific exonuclease encoded by the recJ gene of Escherichia coli. Proc Natl Acad Sci USA 86:2627-2631. https://doi.org/10.1073/pnas.86.8.2627
27. Han E S, Cooper D L, Persky N S, Sutera V A, Whitaker R D, Montello M L, Lovett S T. 2006. RecJ exonuclease: substrates, products and interaction with SSB. Nucleic Acids Res 34:1084-1091. https://doi.org/10.1093/nar/gkj503
28. Midgley-Smith S L, Dimude J U, Rudolph C J. 2019. A role for 3′ exonucleases at the final stages of chromosome duplication in Escherichia coli. Nucleic Acids Res 47:1847-1860. https://doi.org/10.1093/nar/gky1253
29. Morimatsu K, Kowalczykowski S C. 2014. RecQ helicase and RecJ nuclease provide complementary functions to resect DNA for homologous recombination. Proc Natl Acad Sci USA 111: E5133-42. https://doi.org/10.1073/pnas.1420009111
30. Srivastav R, Sharma R, Tandon S, Tandon C. 2019. Role of DHH superfamily proteins in nucleic acids metabolism and stress tolerance in prokaryotes and eukaryotes. Int J Biol Macromol 127:66-75. https://doi.org/10.1016/j.ijbiomac.2018.12.123
31. Mechold U, Fang G, Ngo S, Ogryzko V, Danchin A. 2007. YtqI from Bacillus subtilis has both oligoribonuclease and pAp-phosphatase activity. Nucleic Acids Res 35:4552-4561. https://doi.org/10.1093/nar/gkm462
32. Wang T C. Smith K C. 1988. Different effects of recJ and recN mutations on the postreplication repair of UV-damaged DNA in Escherichia coli K-12. J Bacteriol 170:2555-2559. https://doi.org/10.1128/jb.170.6.2555-2559.1988
33. Lovett S T, Clark A J. 1984. Genetic analysis of the recJ Gene of Escherichia coli K-12. J Bacteriol 157:190-196. https://doi.org/10.1128/jb.157.1.190-196.1984
34. Wang W. Ma L, Wang L. Zheng L, Zheng M. 2020. RecJ from Bacillus halodurans possesses endonuclease activity at moderate temperature. FEBS Lett 594:2303-2310. https://doi.org/10.1002/1873-3468.13809
35. Makarova K S, Koonin E V, Kelman Z. 2012. The CMG (CDC45/RecJ. MCM, GINS) complex is a conserved component of the DNA replication system in all archaea and eukaryotes. Biol Direct 7:7. https://doi.org/10.1186/1745-6150-7-7
36. Kusano K, Sunohara Y, Takahashi N, Yoshikura H, Kobayashi I. 1994. DNA double-strand break repair: genetic determinants of flanking crossingover. Proc Natl Acad Sci USA 91:1173-1177. https://doi.org/10.1073/pnas.91.3.1173
37. Ishino S, Yamagami T, Kitamura M, Kodera N, Mori T, Sugiyama S, Ando T, Goda N, Tenno T, Hiroaki H, Ishino Y. 2014. Multiple interactions of the intrinsically disordered region between the helicase and nuclease domains of the archaeal Hef protein. J Biol Chem 289:21627-21639. https://doi.org/10.1074/jbc.M114.554998
38. Kelman L M, O'Dell W B, Kelman Z. 2020. Unwinding 20 years of the archaeal minichromosome maintenance helicase. J Bacteriol 202: e00729-19. https://doi.org/10.1128/JB.00729-19
39. Oyama T, Ishino S, Shirai T, Yamagami T, Nagata M, Ogino H, Kusunoki M, Ishino Y. 2016. Atomic structure of an archaeal GAN suggests its dual roles as an exonuclease in DNA repair and a CMG component in DNA replication. Nucleic Acids Res 44:9505-9517. https://doi.org/10.1093/nar/gkw789
40. Li Z, Pan M, Santangelo T J, Chemnitz W, Yuan W, Edwards J L, Hurwitz J, Reeve J N, Kelman Z. 2011. A novel DNA nuclease is stimulated by association with the GINS complex. Nucleic Acids Res 39:6114-6123. https://doi.org/10.1093/nar/gkr181
41. Nagata M, Ishino S, Yamagami T, Ogino H, Simons J-R, Kanai T, Atomi H, Ishino Y. 2017. The Cdc45/RecJ-like protein forms a complex with GINS and MCM, and is important for DNA replication in Thermococcus kodakarensis. Nucleic Acids Res 45:10693-10705. https://doi.org/10.1093/nar/gkx740
42. Komori K, Fujikane R, Shinagawa H, Ishino Y. 2002. Novel endonuclease in Archaea cleaving DNA with various branched structure. Genes Genet Syst 77:227-241. https://doi.org/10.1266/ggs.77.227
43. Feng L, Chang C C, Song D, Jiang C, Song Y, Wang C F, Deng W, Zou Y J, Chen H F, Xiao X, Wang F P, Liu X P. 2018. The trimeric Hef-associated nuclease HAN is a 3′->5′ exonuclease and is probably involved in DNA repair. Nucleic Acids Res 46:9027-9043. https://doi.org/10.1093/nar/gky707
44. Lundin C, North M, Erixon K, Walters K, Jenssen D, Goldman A S H, Helleday T. 2005. Methyl methanesulfonate (MMS) produces heat-labile DNA damage but no detectable in vivo DNA double-strand breaks. Nucleic Acids Res 33:3799-3811. https://doi.org/10.1093/nar/gki681
45. Brooks S A. 2010. Functional interactions between mRNA turnover and surveillance and the ubiquitin proteasome system. Wiley Interdiscip Rev RNA 1:240-252. https://doi.org/10.1002/wrna.11
46. Mirsanaye A S. Typas D, Mailand N. 2021. Ubiquitylation at stressed replication forks: mechanisms and functions. Trends Cell Biol 31:584-597. https://doi.org/10.1016/j.tcb.2021.01.008
47. Goetzke C C, Ebstein F, Kallinich T. 2021. Role of proteasomes in inflammation. J Clin Med 10:1783. https://doi.org/10.3390/-jcm10081783
48. Velázquez-Cruz A, Baños-Jaime B. Díaz-Quintana A, De la Rosa M A, Díaz-Moreno I. 2021. Post-translational control of RNA-binding proteins and disease-related dysregulation. Front Mol Biosci 8:658852. https://doi.org/10.3389/fmolb.2021.658852
49. Hochstrasser M. 2000. Evolution and function of ubiquitin-like protein conjugation systems. Nat Cell Biol 2:E153-7. https://doi.org/10.1038/35019643
50. Maupin-Furlow J A. 2014. Prokaryotic ubiquitin-like protein modification. Annu Rev Microbiol 68:155-175. https://doi.org/10.1146/annurev-micro-091313-103447
51. Fu X. Adams Z, Liu R, Hepowit N L, Wu Y, Bowmann C F, Moskovitz J, Maupin-Furlow J A, Gottesman S. 2017. Methionine sulfoxide reductase A (MsrA) and its function in ubiquitin-like protein modification in Archaea. mBio 8:e01169-17. https://doi.org/10.1128/mBio.01169-17
52. Hepowit N L, Maupin-Furlow J A. 2019. Rhodanese-like domain protein UbaC and its role in ubiquitin-like protein modification and sulfur mobilization in Archaea. J Bacteriol 201:e00254-19. https://doi.org/10.1128/JB.00254-19
53. Miranda H V, Nembhard N. Su D, Hepowit N, Krause D J, Pritz J R, Phillips C, Soll D, Maupin-Furlow J A. 2011. E1- and ubiquitin-like proteins provide a direct link between protein conjugation and sulfur transfer in archaea. Proc Natl Acad Sci USA 108:4417-4422. https://doi.org/10.1073/pnas. 1018151108
54. Miranda H V, Antelmann H, Hepowit N, Chavarria N E, Krause D J, Pritz J R, Basell K, Becher D, Humbard M A, Brocchieri L, Maupin-Furlow J A. 2014. Archaeal ubiquitin-like SAMP3 is isopeptide-linked to proteins via a UbaA-dependent mechanism. Mol Cell Proteomics 13:220-239. https://doi.org/10.1074/mcp.M113.029652
55. Bodnar N O, Kim K H, Ji Z, Wales T E, Svetlov V, Nudler E, Engen J R, Walz T, Rapoport T A. 2018. Structure of the Cdc48 ATPase with its ubiquitin-binding cofactor Ufd1-Npl4. Nat Struct Mol Biol 25:616-622. https://doi.org/10.1038/s41594-018-0085-x
56. Huang R, Ripstein Z A, Augustyniak R, Lazniewski M, Ginalski K, Kay L E, Rubinstein J L. 2016. Unfolding the mechanism of the AAA+ unfoldase VAT by a combined cryo-EM, solution NMR study. Proc Natl Acad Sci USA 113:E4190-9. https://doi.org/10.1073/pnas. 1603980113
57. Mallikarjun J, Gowrishankar J. 2022. Essential role for an isoform of Escherichia coli translation initiation factor IF2 in repair of two-ended DNA double-strand breaks. J Bacteriol 204:e0057121. https://doi.org/10.1128/jb.00571-21
58. Marshall C J, Santangelo T J. 2020. Archaeal DNA repair mechanisms. Biomolecules 10:1472. https://doi.org/10.3390/biom10111472
59. Wortz J, Smith V, Fallmann J, Konig S, Thuraisingam T, Walther P, Urlaub H, Stadler P F, Allers T, Hille F. Marchfelder A. 2022. Cas1 and Fen1 display equivalent functions during archaeal DNA repair. Front Microbiol 13:822304. https://doi.org/10.3389/fmicb.2022.822304
60. Brendel J. Stoll B. Lange S J, Sharma K. Lenz C, Stachler A-E. Maier L-K, Richter H, Nickel L, Schmitz R A. Randau L. Allers T, Urlaub H, Backofen R, Marchfelder A. 2014. A complex of Cas proteins 5, 6, and 7 is required for the biogenesis and stability of clustered regularly interspaced short palindromic repeats (CRISPR)-derived RNAs (crRNAs) in Haloferax volcanii. J Biol Chem 289:7164-7177. https://doi.org/10.1074/jbc.M113.508184
61. Chavarria N E, Hwang S. Cao S, Fu X, Holman M, Elbanna D, Rodriguez S, Arrington D, Englert M. Uthandi S, Soll D. Maupin-Furlow J A. 2014. Archaeal Tuc1/Ncs6 homolog required for wobble uridine tRNA thiolation is associated with ubiquitin-proteasome, translation, and RNA processing system homologs. PLOS One 9:e99104. https://doi.org/10.1371/journal.pone.0099104
62. Jumper J. Evans R, Pritzel A, Green T, Figurnov M, Ronneberger O, Tunyasuvunakool K, Bates R, Žídek A, Potapenko A. Bridgland A, Meyer C, Kohl S A A, Ballard A J, Cowie A, Romera-Paredes B, Nikolov S, Jain R, Adler J, Back T, Petersen S, Reiman D. Clancy E, Zielinski M, Steinegger M, Pacholska M, Berghammer T, Bodenstein S, Silver D. Vinyals O, Senior A W, Kavukcuoglu K, Kohli P, Hassabis D. 2021. Highly accurate protein structure prediction with AlphaFold. Nature 596:583-589. https://doi.org/10.1038/s41586-021-03819-2
63. Mevarech M, Frolow F, Gloss L M. 2000. Halophilic enzymes: proteins with a grain of salt. Biophys Chem 86:155-164. https://doi.org/10.1016/s0301-4622(00)00126-5
64. Lanyi J K. 1974. Salt-dependent properties of proteins from extremely halophilic bacteria. Bacteriol Rev 38:272-290. https://doi.org/10.1128/br.38.3.272-290.1974
65. Jia H, Couto-Rodriguez R L, Gal D, Mondragon P, Wassel P C, Yu D, Maupin-Furlow J A. 2021. Expression and tandem affinity purification of 20S proteasomes and other multisubunit complexes in Haloferax volcanii. Methods Enzymol 659:315-326. https://doi.org/10.1016/bs.mie.2021.08.002
66. Melikishvili M, Rodgers D W, Fried M G. 2011. 6-Carboxyfluorescein and structurally similar molecules inhibit DNA binding and repair by 06-alkylguanine DNA alkyltransferase. DNA Repair (Amst) 10:1193-1202. https://doi.org/10.1016/j.dnarep.2011.09.007
67. Schoenberg D R. 2007. The end defines the means in bacterial mRNA decay. Nat Chem Biol 3:535-536. https://doi.org/10.1038/nchembio0907-535
68. Bodnar N, Rapoport T. 2017. Toward an understanding of the Cdc48/p97 ATPase. F1000Res 6:1318. https://doi.org/10.12688/f1000research.11683.1
69. Hänzelmann P, Galgenmüller C, Schindelin H. 2019. Structure and function of the AAA+ ATPase p97, a key player in protein homeostasis. Subcell Biochem 93:221-272. https://doi.org/10.1007/978-3-030-28151-9_7
70. Hepowit N L, de Vera I M S, Cao S, Fu X, Wu Y, Uthandi S, Chavarria N E, Englert M, Su D, Soll D, Kojetin D J, Maupin-Furlow J A. 2016. Mechanistic insight into protein modification and sulfur mobilization activities of noncanonical E1 and associated ubiquitin-like proteins of Archaea. FEBS J 283:3567-3586. https://doi.org/10.1111/febs.13819
71. Koonin E V, Wolf Y I, Aravind L. 2001. Prediction of the archaeal exosome and its connections with the proteasome and the translation and transcription machineries by a comparative-genomic approach. Genome Res 11:240-252. https://doi.org/10.1101/gr.162001
72. Anantharaman V, Aravind L. 2002. The PRC-barrel: a widespread, conserved domain shared by photosynthetic reaction center subunits and proteins of RNA metabolism. Genome Biol 3: RESEARCH0061. https://doi.org/10.1186/gb-2002-3-11-research0061
73. Wilson H L, Aldrich H C, Maupin-Furlow J. 1999. Halophilic 20S proteasomes of the archaeon Haloferax volcanii: Purification, characterization, and gene sequence analysis. J Bacteriol 181:5814-5824.

https://doi.org/10.1128/JB.181.18.5814-5824.1999
74. McMillan L J, Hepowit N L, Maupin-Furlow J A. 2016. Archaeal inorganic pyrophosphatase displays robust activity in organic solvents. Appl Environ Microbiol 82:538-548. under high-salt conditions and https://doi.org/10.1128/AEM.03055-15
75. Schierling K, Rosch S, Rupprecht R, Schiffer S, Marchfelder A. 2002. tRNA 3′ end maturation in archaea has eukaryotic features: the RNase Z from Haloferax volcanii. J Mol Biol 316:895-902. https://doi.org/10.1006/jmbi.2001.5395
76. Portnoy V, Schuster G. 2006. RNA polyadenylation and degradation in different archaea; roles of the exosome and RNase R. Nucleic Acids Res 34:5923-5931. https://doi.org/10.1093/nar/gk1763
77. Matos R G, López-Viñas E, Gomez-Puertas P, Arraiano C M. 2012. The only exoribonuclease present in Haloferax volcanii has an unique response to temperature changes. Biochim Biophys Acta 1820:1543-1552. https://doi.org/10.1016/j.bbagen.2012.06.009
78. Twomey E C, Ji Z, Wales T E, Bodnar N O, Ficarro S B, Marto J A, Engen J R, Rapoport T A. 2019. Substrate processing by the Cdc48 ATPase complex is initiated by ubiquitin unfolding. Science 365: eaax1033. https://doi.org/10.1126/science.aax1033
79. Fu X, Liu R, Sanchez I, Silva-Sanchez C, Hepowit N L, Cao S, Chen S, Maupin-Furlow J, Gottesman S. 2016. Ubiquitin-like proteasome system represents a eukaryotic-like pathway for targeted proteolysis in archaea. mBio 7:e01192-16. https://doi.org/10.1128/mBio.01192-16
80. Barthelme D, Sauer R T. 2013. Bipartite determinants mediate an evolutionarily conserved interaction between Cdc48 and the 20S peptidase. Proc Natl Acad Sci USA 110:3327-3332. https://doi.org/10.1073/pnas.1300408110
81. Barthelme D, Sauer R T. 2012. Identification of the Cdc48.20S proteasome as an ancient AAA+ proteolytic machine. Science 337:843-846. https://doi.org/10.1126/science.1224352
82. Ripstein Z A, Huang R, Augustyniak R, Kay L E, Rubinstein J L. 2017. Structure of a AAA+ unfoldase in the process of unfolding substrate. Elife 6:e25754. https://doi.org/10.7554/eLife.25754
83. Pamnani V, Tamura T, Lupas A, Peters J, Cejka Z, Ashraf W, Baumeister W. 1997. Cloning, sequencing and expression of VAT, a Cdc48/p97 ATPase homologue from the archaeon Thermoplasma acidophilum. FEBS Lett 404:263-268. https://doi.org/10.1016/s0014-5793(97)00138-5
84. Maric M, Maculins T, De Piccoli G, Labib K. 2014. Cdc48 and a ubiquitin ligase drive disassembly of the CMG helicase at the end of DNA replication. Science 346:1253596. https://doi.org/10.1126/science.1253596
85. Hendriks I A, Lyon D, Young C, Jensen L J, Vertegaal A C O. Nielsen M L. 2017. Site-specific mapping of the human SUMO proteome reveals comodification with phosphorylation. Nat Struct Mol Biol 24:325-336. https://doi.org/10.1038/nsmb.3366
86. Hänzelmann P, Schindelin H. 2017. The interplay of cofactor interactions and post-translational modifications in the regulation of the AAA+ ATPase p97. Front Mol Biosci 4:21. https://doi.org/10.3389/fmolb.2017.00021
87. Banerjee S. Bartesaghi A. Merk A, Rao P. Bulfer S L, Yan Y, Green N. Mroczkowski B, Neitz R J. Wipf P, Falconieri V, Deshaies R J, Milne J L S, Huryn D, Arkin M, Subramaniam S. 2016. 2.3 Å resolution cryo-EM structure of human p97 and mechanism of allosteric inhibition. Science 351:871-875. https://doi.org/10.1126/science.aad7974
88. Humbard M A, Reuter C J, Zuobi-Hasona K, Zhou G, Maupin-Furlow J A. 2010. Phosphorylation and methylation of proteasomal proteins of the haloarchaeon Haloferax volcanii. Archaea 2010:481725. https://doi.org/10.1155/2010/481725
89. Figaro S, Durand S, Gilet L, Cayet N, Sachse M, Condon C. 2013. Bacillus subtilis mutants with knockouts of the genes encoding ribonucleases RNase Y and RNase J1 are viable, with major defects in cell morphology, sporulation, and competence. J Bacteriol 195:2340-2348. https://doi.org/10.1128/JB.00164-13
90. Linder P, Lemeille S, Redder P. 2014. Transcriptome-wide analyses of 5′-Ends in RNase J mutants of a gram-positive pathogen reveal a role in RNA maturation, regulation and degradation. PLOS Genet 10:e1004207. https://doi.org/10.1371/journal.pgen.1004207
91. Tzafrir I, Pena-Muralla R, Dickerman A, Berg M, Rogers R, Hutchens S, Sweeney T C, McElver J, Aux G, Patton D, Meinke D. 2004. Identification of genes required for embryo development in Arabidopsis. Plant Physiol 135:1206-1220. https://doi.org/10.1104/pp.104.045179
92. Cucinotta C E, Hildreth A E, McShane B M, Shirra M K, Arndt K M. 2019. The nucleosome acidic patch directly interacts with subunits of the Paf1 and FACT complexes and controls chromatin architecture in vivo. Nucleic Acids Res 47:8410-8423. https://doi.org/10.1093/nar/gkz549
93. Lehnik-Habrink M, Lewis R J, Mäder U, Stülke J. 2012. RNA degradation in Bacillus subtilis: an interplay of essential endo- and exoribonucleases. Mol Microbiol 84:1005-1017. https://doi.org/10.1111/j.1365-2958.2012.08072.x
94. Redko Y, Aubert S, Stachowicz A, Lenormand P, Namane A, Darfeuille F, Thibonnier M, De Reuse H. 2013. A minimal bacterial RNase J-based degradosome is associated with translating ribosomes. Nucleic Acids Res 41:288-301. https://doi.org/10.1093/nar/gks945
95. Cho K H. 2017. The structure and function of the gram-positive bacterial RNA degradosome. Front Microbiol 8:154. https://doi.org/10.3389/fmicb.2017.00154
96. Cui T, Zhang L, Wang X, He Z-G. 2009. Uncovering new signaling proteins and potential drug targets through the interactome analysis of Mycobacterium tuberculosis. BMC Genomics 10:118. https://doi.org/10.1186/1471-2164-10-118
97. Sun L, Zhang L. Zhang H, He Z G. 2011. Characterization of a bifunctional β-Lactamase/ribonuclease and its interaction with a chaperone-like protein in the pathogen Mycobacterium tuberculosis H37Rv. Biochemistry (Mosc) 76:350-358. https://doi.org/10.1134/-s0006297911030096
98. Cascante-Estepa N, Gunka K, Stülke J. 2016. Localization of components of the RNA-degrading machine in Bacillus subtilis. Front Microbiol 7:1492. https://doi.org/10.3389/fmicb.2016.01492
99. Liu J. Wang Q, Jiang X, Yang H, Zhao D, Han J, Luo Y, Xiang H. 2017. Systematic analysis of lysine acetylation in the halophilic archaeon Haloferax mediterranei. J Proteome Res 16:3229-3241. https://doi.org/10.1021/acs.jproteome.7b00222
100. Couto-Rodríguez R L, Koh J. Chen S, Maupin-Furlow J A. 2023. Insights into the lysine acetylome of the haloarchaeon Haloferax volcanii during oxidative stress by quantitative SILAC-based proteomics. Antioxidants 12:1203. https://doi.org/10.3390/antiox12061203
101. Portnoy V, Evguenieva-Hackenberg E, Klein F, Walter P, Lorentzen E, Klug G, Schuster G. 2005. RNA polyadenylation in Archaea: not observed in Haloferax while the exosome polynucleotidylates RNA in Sulfolobus. EMBO Rep 6:1188-1193. https://doi.org/10.1038/sj.embor.7400571
102. Dreyfus M, Régnier P. 2002. The poly(A) tail of mRNAs: bodyguard in eukaryotes, scavenger in bacteria. Cell 111:611-613. https://doi.org/10.1016/s0092-8674(02)01137-6
103. Oren A. 2013. Life at high salt concentrations, intracellular KCl concentrations, and acidic proteomes. Front Microbiol 4:315. https://doi.org/10.3389/fmicb.2013.00315
104. Bader A S, Bushell M. 2020. DNA:RNA hybrids form at DNA double-strand breaks in transcriptionally active loci. Cell Death Dis 11:280. https://doi.org/10.1038/s41419-020-2464-6
105. Schulze S, Adams Z, Cerletti M, De Castro R, Ferreira-Cerca S, Fufezan C, Giménez M I, Hippler M, Jevtic Z, Knüppel R, Legerme G, Lenz C, Marchfelder A, Maupin-Furlow J, Paggi R A, Pfeiffer F, Poetsch A, Urlaub H, Pohlschroder M. 2020. The Archaeal Proteome Project advances knowledge about archaeal cell biology through comprehensive proteomics. Nat Commun 11:3145. https://doi.org/10.1038/s41467-020-16784-7
106. Dyall-Smith M. 2009. The Halohandbook: Protocols for Halobacterial Genetics, vol Version 7.2, http://www.haloarchaea.com/resources/halohandbook/Halohandbook_2009_v7.2mds.pdf.
107. Allers T, Ngo H P, Mevarech M, Lloyd R G. 2004. Development of additional selectable markers for the halophilic archaeon Haloferax volcanii based on the leuB and trpA genes. Appl Environ Microbiol 70:943-953. https://doi.org/10.1128/AEM.70.2.943-953.2004
108. Ho S N, Hunt H D, Horton R M, Pullen J K, Pease L R. 1989. Site-directed mutagenesis by overlap extension using the polymerase chain reaction. Gene 77:51-59. https://doi.org/10.1016/0378-1119(89)90358-2
109. Allers T, Barak S, Liddell S, Wardell K, Mevarech M. 2010. Improved strains and plasmid vectors for conditional overexpression of His-tagged proteins in Haloferax volcanii. Appl Environ Microbiol 76:1759-1769. https://doi.org/10.1128/AEM.02670-09
110. Bab-Dinitz E, Shmuely H, Maupin-Furlow J, Eichler J, Shaanan B. 2006. Haloferax volcanii PitA: an example of functional interaction between the Pfam chlorite dismutase and antibiotic biosynthesis monooxygenase families. Bioinformatics 22:671-675. https://doi.org/10.1093/bioinformatics/btk043
111. Oren A. 2008. Microbial life at high salt concentrations: phylogenetic and metabolic diversity. Saline Syst 4:2. https://doi.org/10.1186/1746-1448-4-2
112. Schneider C A, Rasband W S, Eliceiri K W. 2012. NIH Image to ImageJ: 25 years of image analysis. Nat Methods 9:671-675. https://doi.org/10.1038/nmeth.2089
113. Keller A, Nesvizhskii A I, Kolker E, Aebersold R. 2002. Empirical statistical model to estimate the accuracy of peptide identifications made by MS/MS and database search. Anal Chem 74:5383-5392. https://doi.org/10.1021/ac025747h
114. Nesvizhskii A I, Keller A, Kolker E, Aebersold R. 2003. A statistical model for identifying proteins by tandem mass spectrometry. Anal Chem 75:4646-4658. https://doi.org/10.1021/ac0341261
115. Searle B C. 2010. Scaffold: a Bioinformatic tool for validating MS/MSbased proteomic studies. Proteomics 10:1265-1269. https://doi.org/10.1002/pmic.200900437
116. Holman S W, Sims P F G, Eyers C E. 2012. The use of selected reaction monitoring in quantitative proteomics. Bioanalysis 4:1763-1786. https://doi.org/10.4155/bio.12.126
117. The UniProt Consortium. 2017. Uniprot: the universal protein knowledgebase. Nucleic Acids Res 45: D158-D169. https://doi.org/10.1093/nar/gkw1099
118. Gerlt J A, Bouvier J T, Davidson D B, Imker H J, Sadkhin B. Slater D R, Whalen K L. 2015. Enzyme Function Initiative-Enzyme Similarity Tool (EFI-EST): a web tool for generating protein sequence similarity networks. Biochim Biophys Acta 1854:1019-1037. https://doi.org/10.1016/j.bbapap.2015.04.015
119. Vizcaíno J A, Csordas A, del-Toro N, Dianes J A, Griss J, Lavidas I, Mayer G, Perez-Riverol Y, Reisinger F, Ternent T, Xu Q-W, Wang R, Hermjakob H. 2016. 2016 update of the PRIDE database and its related tools. Nucleic Acids Res 44:11033. https://doi.org/10.1093/nar/gkw880

What is claimed is:

1. A nuclease complex system comprising a nuclease and a set of proteins for controlling the nuclease to control the degradation of a single stranded nucleic acid, wherein the nuclease is aRNase J, wherein the set of nuclease-controlling proteins comprises RecJ3 and RecJ4, and wherein the single stranded nucleic acid is RNA or ssDNA.

2. The nuclease complex system of claim 1, wherein aRNase J has an amino acid sequence of SEQ ID NO:5 or any RNase with sequence identity above 90% thereof.

3. The nuclease complex system of claim 2, wherein aRNase J forms a homodimer, and wherein aRNase J is isolated from archaea that do not encode a canonical RNA exosome,
selected from Euryarchaeota comprising thermococcales, methanosarcinales, methanomicrobiales, haloarchaea and certain methanogens, and optionally Haloferax volcanii.

4. The nuclease complex system of claim 1, wherein RecJ3 and RecJ4 complex forms a heterodimer at an equimolar ratio.

5. The nuclease complex system of claim 4, wherein RecJ3 comprises an amino acid sequence of SEQ ID NO:1 or any amino acid sequence with sequence identity above 90% thereof.

6. The nuclease complex system of claim 4 or 5, wherein RecJ3 is isolated from archaea,
selected from Euryarchaeota comprising thermococcales, methanosarcinales, methanomicrobiales, haloarchaea and certain methanogens, and optionally H. volcanii.

7. The nuclease complex system of claim 4, wherein RecJ4 comprises an amino acid sequence of SEQ ID NO:3 or any amino acid sequence with sequence identity above 90% thereof.

8. The nuclease complex system of claim 4 or 7, wherein RecJ4 is isolated from archaea,
selected from Euryarchaeota comprising thermococcales, methanosarcinales, methanomicrobiales, haloarchaea and certain methanogens, and optionally H. volcanii.

9. The nuclease complex system of any one of the preceding claims,
wherein the ratio of RecJ3, RecJ4 and aRNase J is within a range of about 0:0:1 to about 2:2:1, and optionally about 0.5:0.5:1 or about 2:2:1,
wherein (i) at the ratio of 0:0:1 of RecJ3, RecJ4, and aRNase J, aRNase J has activity as endonuclease and 5′-3′ exonuclease; (ii) at a low ratio, the complex has more activity as endonuclease and 5′-3′ exonuclease than 3′-5′ exonuclease, (iii) at a high ratio, the complex has more activity as 3′-5′ exonuclease than endonuclease and 5′-3′ exonuclease; and (iv) at the ratio of 2:2:1, the complex has 3′-5′ exonuclease activity.

10. The nuclease complex system of claim 3, wherein aRNase J optionally has a tag at its C-terminal or N-terminal for protein purification, wherein the tag can be selected from hexa-histidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, and the like.

11. The nuclease complex system of claim 10, wherein aRNase J has StrepII tag at its C-terminal.

12. The nuclease complex system of claim, wherein RecJ3 optionally has a tag at its C-terminal or N-terminal, wherein the tag can be selected from hexa-histidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, and the like.

13. The nuclease complex system of claim, wherein RecJ3 has His tag at its N terminal.

14. The nuclease complex system of claim, wherein RecJ3 optionally has a tag at its C-terminal or N-terminal, wherein the tag can be selected from hexa-histidine (6× His), glutathione S-transferase (GST), FLAG, streptavidin-binding peptide (SBP), Strep II, maltose-binding protein (MBP), calmodulin-binding peptide (CBP), chitin-binding domain (CBD), HA, c-Myc, and the like.

15. The nuclease complex system of claim, wherein RecJ4 has StrepII tag at its C terminal.

16. An in vitro nuclease activity titration kit comprising RecJ3, RecJ4, and aRNaseJ proteins individually packed in a container as powder or concentrated solution form, which are at least partially purified, wherein the kit further comprises concentrated reaction buffer having pH of about 7.2-7.7, Mg²⁺, Mn²⁺, and high salt to make a final concentration of about 2M NaCl in the reaction solution, reaction stop solution comprising EDTA and/or TPEN, and nuclease activity titration substrates comprising RNAs or ssDNA labeled with 6-carboxyfluorescein (6-FAM) at 5′ end or 3′ end, and RNAs labeled internally with phosphorothioate and at 3′ end with 6-FAM.

17. An in vivo nuclease complex system expressing aRNase J homodimer and RecJ3/4 heterodimers according to claims 1 to 15, comprising
a recombinant expression vector comprising a nucleic acid strand encoding both RecJ3 of SEQ ID NO:2 and RecJ4 of SEA ID NO: 4 or any nucleic acid sequences with sequence identity above 90% thereof, wherein a promoter can be positioned upstream of the nucleic acid strand encoding both RecJ3 and RecJ4, and wherein the promoter is inducible by tryptophan, IPTG or arabinose, and
a recombinant expression vector comprising a nucleic acid strand encoding aRNase J of SEA ID NO: 6 or any nucleic acid sequence with sequence identity above 90% thereof, wherein a promoter, inducible or constitutively active, different from said promoter can be positioned upstream of the nucleic acid strand encoding aRNase J,
or
a recombinant expression vector comprising a nucleic acid strand encoding all three proteins of RecJ3, RecJ4 and aRNaseJ, wherein a promoter can be positioned upstream of the nucleic acid strand encoding both RecJ3 and RecJ4, wherein the promoter is an inducible by tryptophan, IPTG, or arabinose, and another promoter, inducible or constitutively active, different from said promoter can be positioned upstream of the nucleic acid strand encoding aRNaseJ.

18. A cell recombinantly engineered to express a heterologous nucleic acid strand encoding RecJ3, RecJ4 and aRNase J, or any of heterologous nucleic acid strands encoding RecJ3, RecJ4 or aRNase J, or a combination thereof according to claim 17, wherein the cell is bacterial, yeast, insect, or mammalian cells.

19. A nucleic acid strand encoding aRNase J with a StrepII tag at its C-terminal (aRNase J-StrepII).

20. A recombinant replication and/or expression vector comprising the nucleic acid strand of claim 19.

21. The recombinant vector of claim 20 is plasmid pJAM1406 (+rnj-strepII).

22. A nucleic acid strand encoding RecJ3 with a His6 tag at its N-terminal (His6-RecJ3).

23. A recombinant replication and/or expression vector comprising the nucleic acid strand of claim 22.

24. The recombinant vector of claim 23 is plasmid pJAM4251 (+his6-recJ3).

25. A recombinant replication and/or expression vector comprising the nucleic acid strand of claims 19 and 22, wherein the recombinant vector is plasmid pJAM4252 (+his6-recJ3 and +rnj-strepII).

26. A nucleic acid strand encoding recJ4 with a StrepII tag at its C-terminal (RecJ4-StrepII).

27. A recombinant replication and/or expression vector comprising the nucleic acid strand of claim 25.

28. The recombinant vector of claim 20 is plasmid pJAM1405 (+recj4-strepII).

29. The nuclease complex system of claim 1, 4, or 5, wherein RecJ3/4 heterodimer is purified by the method comprising steps of:
lysing H. volcanii HJ02-pJAM4251 expressing His-RecJ3 to produce cell lysate,
clarifying the cell lysate by PEG 8000 addition, centrifugation and filtration,
dialyzing the cell lysate,
performing a HisTrap HP chromatography with the dialyzed cell lysate, and
optionally performing a size exclusion chromatography.

30. The nuclease complex system of claim 1 or 4, wherein RecJ3/4 heterodimer and RecJ3/4-aRNase J complex are purified by the method comprising steps of;
lysing H. volcanii HJ02-pJAM4252 or HJ07-pJAM4252 expressing His-RecJ3 and aRNase J-StrepII to produce cell lysate,
clarifying the cell lysate by PEG 8000 addition, centrifugation and filtration,
dialyzing the cell lysate,
performing a HisTrap HP chromatography with the dialyzed cell lysate to obtain RecJ3/4 heterodimer,
performing a Strep-Tactin chromatography with the flowthrough from the HisTrap HP chromatography to obtain aRNase J,
Optionally performing a size exclusion chromatography with the elution from the His Trap chromatography or the Strep-Tactin chromatography.

31. The nuclease complex system of any one of claims 1 to 3, wherein aRNase J homodimer is purified by the method comprising steps of:
lysing H. volcanii HS01-pJAM1406 expressing aRNase J-StrepII to produce a lysate,
clarifying the cell lysate by PEG 8000 addition, centrifugation and filtration,
dialyzing the cell lysate,
performing a Strep-Tactin chromatography, and
optionally performing a size exclusion chromatography.

32. The nuclease complex system of claim 1, 4, or 8, wherein RecJ4 is purified by the method comprising steps of:
lysing H. volcanii H26-pJAM1405 expressing Recj4-StrepII to produce a lysate,
clarifying the cell lysate by PEG 8000 addition, centrifugation and filtration,
dialyzing the cell lysate,
performing a Strep-Tactin chromatography, and
optionally performing a size exclusion chromatography.

33. A method of controlling direction of nucleotide degradation in a sample comprising a single-stranded DNA or RNA, the method comprising providing to the sample an amount of (i) RecJ3/J4 RNase from Haloferax volcanii and/or (ii) aRNase J from Haloferax volcanii; wherein 5′-3′ nucleotide degradation is increased by providing a lower ratio of (i) to (ii) and 3′-5′ nucleotide degradation is increased by providing a higher ratio of (i) to (ii).
